(19) United States
(12) Patent Application Publication
(10) Pub. No.: US 2024/0400707 A1
(43) Pub. Date: Dec. 5, 2024

(54) COMPOSITIONS AND METHODS OF TREATING FACIOSCAPULOHUMERAL MUSCULAR DYSTROPHY

(71) Applicant: Avidity Biosciences, Inc., San Diego, CA (US)

(72) Inventors: Barbora MALECOVA, San Diego, CA (US); Rob BURKE, Carlsbad, CA (US); Beatrice Diana DARIMONT, San Diego, CA (US); David SALA CANO, San Diego, CA (US)

(21) Appl. No.: 18/809,215

(22) Filed: Aug. 19, 2024

Related U.S. Application Data
(63) Continuation of application No. 18/402,604, filed on Jan. 2, 2024.
Application No. 18/402,604 is a division of application No. 17/932,653, filed on Sep. 15, 2022, now Pat. No. 11,912,779.
(60) Provisional application No. 63/245,123, filed on Sep. 16, 2021.

(51) Int. Cl. C07K 16/28; A61K 39/00; A61P 21/00; C12N 15/113

(52) U.S. Cl. CPC C07K 16/2881; A61P 21/00; C12N 15/113; A61K 2039/505; C12N 2310/141; C12N 2310/315; C12N 2310/321

(57) ABSTRACT

Disclosed herein are polynucleic acid molecules, pharmaceutical compositions, and methods for treating facioscapulohumeral muscular dystrophy (FSHD).

### CROSS-REFERENCE

This application is a Continuation of U.S. Non-provisional application Ser. No. 18/402,604, filed Jan. 2, 2024, which is a Divisional of U.S. Non-Provisional application Ser. No. 17/932,653, filed Sep. 15, 2022, nowissued as U.S. Pat. No. 11,912,779, on Feb. 27, 2024, which claims the benefit of U.S. Provisional Application No. 63/245,123, filed Sep. 16, 2021, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Aug. 15, 2024, is named 45532-756_301_SL.xml and is 2,155,539 bytes in size.

### BACKGROUND OF THE DISCLOSURE

Muscle atrophy is the loss of muscle mass or the progressive weakening and degeneration of muscles, such as skeletal or voluntary muscles that controls movement, cardiac muscles, and smooth muscles. Various pathophysiological conditions including disuse, starvation, cancer, diabetes, and renal failure, or treatment with glucocorticoids result in muscle atrophy and loss of strength. The phenotypical effects of muscle atrophy are induced by various molecular events, including inhibition of muscle protein synthesis, enhanced turnover of muscle proteins, abnormal regulation of satellite cells differentiation, and abnormal conversion of muscle fibers types.

FSHD is a rare, progressive and disabling disease for which there are no approved treatments. FSHD is one of the most common forms of muscular dystrophy and affects both sexes equally, with onset typically in teens and young adults. FSHD is characterized by progressive skeletal muscle loss that initially causes weakness in muscles in the face, shoulders, arms and trunk and progresses to weakness in muscles in lower extremities and the pelvic girdle. Skeletal muscle weakness results in significant physical limitations, including progressive loss of facial muscles that can cause an inability to smile or communicate, difficulty using arms for activities of daily living and difficulty getting out of bed, with many patients ultimately becoming dependent upon the use of a wheelchair for daily mobility activities. The majority of patients with FSHD also report experiencing chronic pain, anxiety and depression.

FSHD is caused by aberrant expression of a gene, DUX4, in skeletal muscle resulting in the inappropriate presence of DUX4 protein. Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long lasting effect over multiple cell divisions. As such, RNAi represents a viable method useful for drug target validation, gene function analysis, pathway analysis, and disease therapeutics.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

Described herein, in some aspects, is a polynucleic acid molecule conjugate comprising: an antibody or antigen binding fragment thereof conjugated to a polynucleic acid molecule that hybridizes to a target sequence of DUX4; wherein the polynucleic acid molecule comprises a nucleic acid sequence that is at least 80%, at least 85%, at least 90%, at least 95%, or 100% identical to a sequence selected from SEQ ID NOs: 72, 76, 126, or 131-136; wherein the polynucleic acid molecule comprises 2′-F modified nucleotides at positions 2, 6, 14, and 16; and wherein the polynucleic acid molecule conjugate mediates RNA interference against the DUX4.

In some embodiments, the antibody or antigen binding fragment thereof comprises a non-human antibody or antigen binding fragment thereof, a human antibody or antigen binding fragment thereof, a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof. In some embodiments, the antibody or antigen binding fragment thereof is an anti-transferrin receptor antibody or antigen binding fragment thereof. In some embodiments, the polynucleic acid molecule is from about 16 to about 30 nucleotides in length. In some embodiments, the polynucleic acid molecule comprises a sense strand and an antisense strand, and the antisense strand comprises a nucleic acid sequence of at least one of UfsNfsnnnNfnnnnnnnNfnNfnnnsusu, usNfsnnnNfnnnnnnnNfnNfnnnsusu, or vpNsNfsnnnNfnnnnnnnNfnNfnnnsus, wherein vpN=vinyl phosphonate VpUq, lower case (n)=2′-O-Me modified, Nf=2′-F modified, and s=phosphorothioate backbone modification. In some embodiments, the polynucleic acid molecule comprises a sense strand and an antisense strand, and the antisense strand comprises a nucleic acid sequence that is at least 80%, at least 85%, at least 90%, at least 95%, or 100% identical to a sequence selected from SEQ ID NOs: 412-420 or 430-438. In some embodiments, the polynucleic acid molecule comprises a sense strand and an antisense strand, and the sense strand comprises a nucleic acid sequence that is at least 80%, at least 85%, at least 90%, at least 95%, or 100% identical to a sequence selected from SEQ ID NOs: 2, 6, 56, or 61-66, wherein the sense strand comprises at least 2 or at least 3 consecutive 2′-F modified nucleotides. In some embodiments, the polynucleic acid molecule comprises a sense strand and an antisense strand, and the sense strand comprises a nucleic acid sequence that is at least 80%, at least 85%, at least 90%, at least 95%, or 100% identical to a sequence selected from SEQ ID NOs: 2, 6, 56, or 61-66. In some embodiments, the polynucleic acid molecule comprises a phosphorothioate linkage or a phosphorodithioate linkage. In some embodiments, the polynucleic acid molecule comprises six or more 2′ modified nucleotides selected from 2′-O-methyl and 2′-deoxy-2′-fluoro. In some embodiments, the polynucleic acid molecule comprises a 5′-terminal vinylphosphonate modified nucleotide. In some embodiments, the 5′-terminal vinylphosphonate modified nucleotide is selected from:

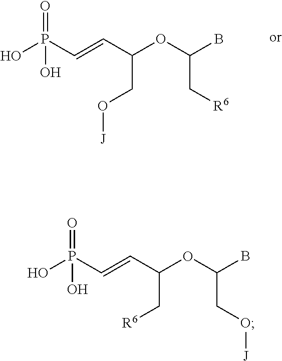

where B is a heterocyclic base moiety; R6 is selected from hydrogen, halogen, alkyl or alkoxy; and J is an internucleotide linking group linking to the adjacent nucleotide of the polynucleic acid molecule. In some embodiments, the sense strand and antisense strand comprises at least two, three, or four consecutive 2′-O-methyl modified nucleotides at the 5′-end or 3′-end. In some embodiments, the polynucleic acid molecule conjugate comprises a linker connecting the antibody or antigen binding fragment thereof to the polynucleic acid molecule via a cysteine residue or a lysine residue on the antibody or antigen binding fragment thereof. In some embodiments, the linker is a C₁-C₆ alkyl linker. In some embodiments, the linker is a homobifunctional linker or heterobifunctional linker, and comprises a maleimide group, a dipeptide moiety, a benzoic acid group, or its derivative thereof. In some embodiments, the linker is a cleavable or non-cleavable linker. In some embodiments, the polynucleic acid molecule conjugate comprises a ratio between the polynucleic acid molecule and the antibody or antigen binding fragment thereof is about 1:1, 2:1, 3:1, or 4:1.

Described herein, in some aspects, is a method for treating muscular dystrophy in a subject in need thereof, comprising: providing a polynucleic acid conjugate as described herein; and administering the polynucleic acid conjugate to the subject in need thereof to treat the muscular dystrophy, wherein the polynucleic acid conjugate reduces a quantity of the mRNA transcript of human DUX4. In some embodiments, the polynucleic acid conjugate mediates RNA interference against the human DUX4 and modulates muscle dystrophy in the subject. In some embodiments, the RNA interference comprises reducing expression of the mRNA transcript of DUX4 gene by at least 50%, at least 60%, or at least 70% or more compared to a quantity of the mRNA transcript of DUX4 gene in an untreated cell. In some embodiments, the RNA interference comprises affecting expression of a marker gene selected from a group consisting of MBD3L2, TRIM43, PRAMEF1, ZSCAN4, KHDC1L, LEUTX, WFDC3, ILVBL, SLC15A2, and SORD in a cell affected by the muscle dystrophy. In some embodiments, the affecting expression comprises reducing expression of the marker gene by at least 20%, at least 30%, at least 40%, at least 50%, at least 60% or more in the cell. In some embodiments, the muscular dystrophy is Facioscapulohumeral muscular dystrophy (FSHD).

Described herein, in some aspects, is a double-stranded polynucleic acid molecule that mediates RNA interference against DUX4, wherein the double-stranded polynucleic acid molecule comprises a sense strand and an antisense strand, wherein the antisense strand comprises a nucleic acid sequence that is at least 80%, at least 85%, at least 90%, at least 95%, or 100% identical to a sequence selected from SEQ ID NOs: 412-420 or 430-438; and the sense strand comprises a nucleic acid sequence that is at least 80%, at least 85%, at least 90%, at least 95%, or 100% identical to a sequence selected from SEQ ID NOs: 142, 146, 196, or 201-206.

Described herein, in some aspects, is a double-stranded polynucleic acid molecule that mediates RNA interference against DUX4, wherein the double-stranded polynucleic acid molecule comprises a sense strand and an antisense strand, wherein the antisense strand comprises a nucleic acid sequence comprising at least 15 contiguous nucleotides differing by no more than 1, 2, 3 nucleotides from a sequence selected from SEQ ID NOs: 412-420 or 430-438; and the sense strand comprises at least 15 contiguous nucleotides differing by no more than 1, 2, 3 nucleotides from a sequence selected from SEQ ID NOs: 142, 146, 196, or 201-206.

Disclosed herein, in certain aspects, are polynucleic acid molecules and pharmaceutical compositions for modulating a gene associated with muscle atrophy, especially Facioscapulohumeral muscular dystrophy (FSHD). In some aspects, also described herein are methods of treating muscle atrophy, especially FSHD, with a polynucleic acid molecule or a polynucleic acid molecule conjugate disclosed herein.

Disclosed herein, in certain aspects, is a polynucleic acid molecule conjugate comprising an antibody or antigen binding fragment thereof conjugated to a polynucleic acid molecule that hybridizes to a target sequence of DUX4, and the polynucleic acid molecule conjugate mediates RNA interference against the DUX4. In certain aspects, the antibody or antigen binding fragment thereof comprises a non-human antibody or binding fragment thereof, a human antibody or antigen binding fragment thereof, a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof. In certain aspects, the antibody or antigen binding fragment thereof is an anti-transferrin receptor antibody or antigen binding fragment thereof.

In certain aspects, the polynucleic acid molecule comprises a sense strand and/or an antisense strand, and wherein the sense strand and/or the antisense strand each independently comprises at least one 2′ modified nucleotide, at least one modified internucleotide linkage, or at least one inverted abasic moiety. In certain aspects, the polynucleotide hybridizes to at least 8 contiguous bases of the target sequence of DUX4. In certain aspects, the polynucleotide is from about 8 to about 50 nucleotides in length or from about 10 to about 30 nucleotides in length. In certain aspects, the polynucleic acid molecule comprises a sense strand and/or an antisense strand, and the sense strand comprises at least 80%, at least 85%, at least 90%, at least 95%, at least 96%, at least 97%, at least 98%, at least 99% identical to a sequence selected from SEQ ID NOs: 1-70 or SEQ ID NOs: 141-210. Alternatively and/or additionally, the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 71-140 or SEQ ID NOs: 211-280. Alternatively and/or additionally, the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 142, 146, 196, 201-206, 412-420, or 430-438. In some embodiments, the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 412-420 or 430-438. In some embodiments, the polynucleic acid molecule comprises a sense strand and/or an antisense strand, and the antisense is identical to a sequence selected from SEQ ID NOs: 412-420 or 430-438. In some embodiments, the polynucleic acid molecule comprises a sense strand and/or an antisense strand, and the sense strand comprises at least 80%, at least 85%, at least 90%, at least 95%, at least 96%, at least 97%, at least 98%, at least 99% identical to a sequence selected from SEQ ID NOs: 142, 146, 196, or 201-206. In some embodiments, the polynucleic acid molecule comprises a sense strand and/or an antisense strand, and the sense strand is identical to a sequence selected from SEQ ID NOs: 142, 146, 196, or 201-206.

In certain aspects, the polynucleotide comprises at least one 2′ modified nucleotide, and further the 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide, or comprises locked nucleic acid (LNA) or ethylene nucleic acid (ENA), or comprises a combination thereof. In certain aspects, the at least one modified internucleotide linkage comprises a phosphorothioate linkage or a phosphorodithioate linkage. In certain aspects, the polynucleic acid molecule comprises three or more 2′ modified nucleotides selected from 2′-O-methyl and 2′-deoxy-2′-fluoro. In certain aspects, the polynucleic acid molecule comprises a 5′-terminal vinylphosphonate modified nucleotide.

Another embodiment provides the polynucleic acid molecule of the polynucleic acid molecule conjugate, wherein the at least one 5′-vinylphosphonate modified non-natural nucleotide is selected from:

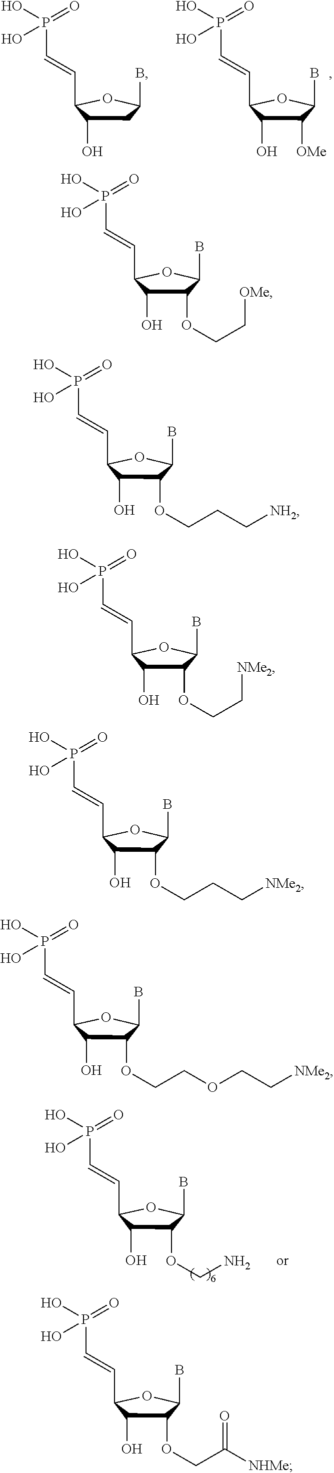

where B is a heterocyclic base moiety.

Another embodiment provides the polynucleic acid molecule of the polynucleic acid molecule conjugate, wherein the at least one 5′-vinylphosphonate modified non-natural nucleotide is selected from:

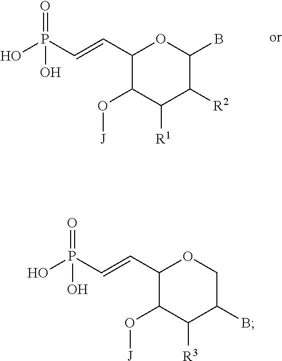

where B is a heterocyclic base moiety; R1, R2, and R3 are independently selected from hydrogen, halogen, alkyl or alkoxy; and J is an internucleotide linking group linking to the adjacent nucleotide of the polynucleotide.

Another embodiment provides the polynucleic acid molecule of the polynucleic acid molecule conjugate, wherein the at least one 5′-vinylphosphonate modified non-natural nucleotide is selected from:

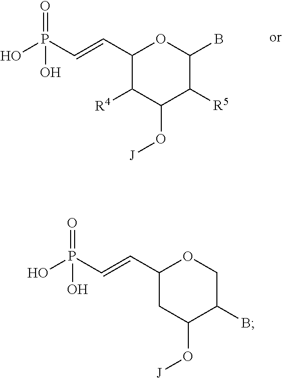

where B is a heterocyclic base moiety; R4, and R5 are independently selected from hydrogen, halogen, alkyl or alkoxy; and J is an internucleotide linking group linking to the adjacent nucleotide of the polynucleotide.

Another embodiment provides the polynucleic acid molecule of the polynucleic acid molecule conjugate, wherein the at least one 5′-vinylphosphonate modified non-natural nucleotide is selected from:

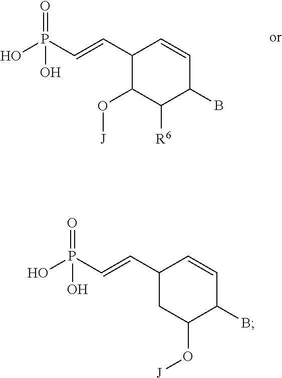

where B is a heterocyclic base moiety; R6 is selected from hydrogen, halogen, alkyl or alkoxy; and J is an internucleotide linking group linking to the adjacent nucleotide of the polynucleotide.

Another embodiment provides the polynucleic acid molecule of the polynucleic acid molecule conjugate, wherein the at least one 5′-vinylphosphonate modified non-natural nucleotide is selected from locked nucleic acid (LNA) or ethylene nucleic acid (ENA).

Another embodiment provides the polynucleic acid molecule of the polynucleic acid molecule conjugate, wherein the at least one 5′-vinylphosphonate modified non-natural nucleotide is selected from:

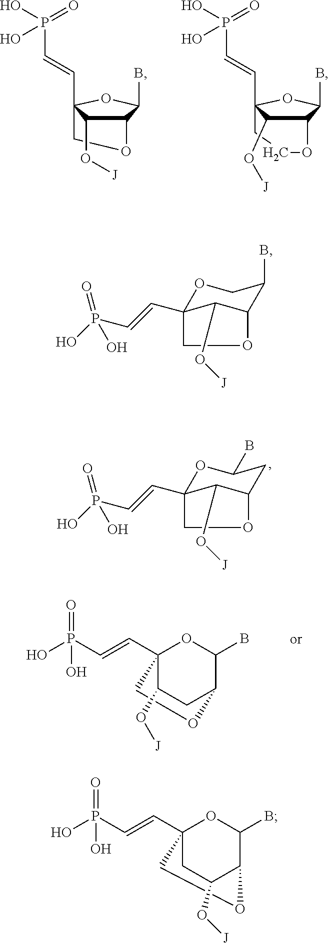

where B is a heterocyclic base moiety; and J is an internucleotide linking group linking to the adjacent nucleotide of the polynucleotide.

Another embodiment provides the polynucleic acid molecule of the polynucleic acid molecule conjugate, wherein the at least one 5′-vinylphosphonate modified non-natural nucleotide is selected from:

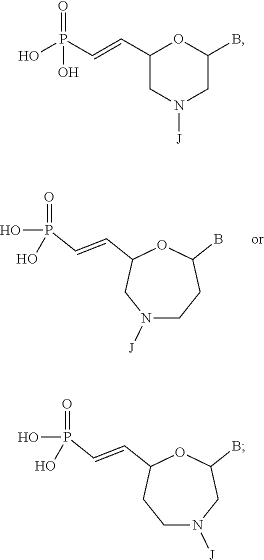

where B is a heterocyclic base moiety; and J is an internucleotide linking group linking to the adjacent nucleotide of the polynucleotide.

Another embodiment provides the polynucleic acid molecule of the polynucleic acid molecule conjugate, wherein the at least one 5′-vinylphosphonate modified non-natural nucleotide is selected from:

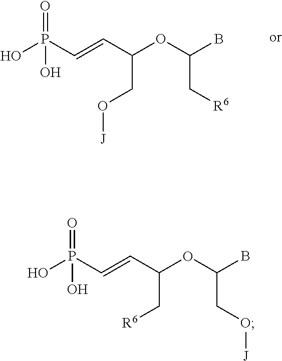

where B is a heterocyclic base moiety; R6 is selected from hydrogen, halogen, alkyl or alkoxy; and J is an internucleotide linking group linking to the adjacent nucleotide of the polynucleotide.

Another embodiment provides the polynucleic acid molecule of the polynucleic acid molecule conjugate, wherein the at least one 5′-vinylphosphonate modified non-natural nucleotide is:

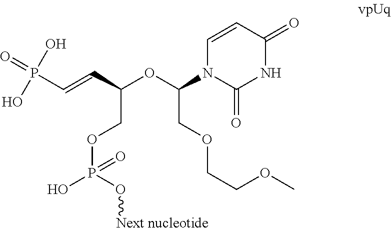

In certain aspects, the 2′ modified nucleotide is 2′-O-methyl modified nucleotide, and 2′-O-methyl modified nucleotide is at the 5′-end of the sense strand and/or the antisense strand. In some aspects, the 2′-O-methyl modified nucleotide is a purine nucleotide, or the 2′-O-methyl modified nucleotide is a pyridine nucleotide. In certain aspects, the sense and/or antisense strands comprise at least two, three, four consecutive the 2′-O-methyl modified nucleotides at the 5′-end.

In certain aspects, the polynucleic acid molecule conjugate comprises a linker connecting the target cell binding moiety to the polynucleic acid moiety. In such aspects, the linker is C₁-C₆ alkyl linker, or the linker is a homobifunctional linker or heterobifunctional linker, and comprises a maleimide group, a dipeptide moiety, a benzoic acid group, or its derivative thereof. Alternatively and/or additionally, the linker is a cleavable or non-cleavable linker. In certain aspects, a ratio between the polynucleic acid moiety and the target cell binding moiety is about 1:1, 2:1, 3:1, or 4:1.

In certain aspects, the polynucleic acid moiety mediates RNA interference against the human DUX4 and modulates symptoms of muscle dystrophy or atrophy in a subject. In some aspects, the RNA interference comprises reducing expression of the mRNA transcript of DUX4 gene at least 50%, at least 60%, or at least 70% or more compared to a quantity of the mRNA transcript of DUX4 gene in an untreated cell. Alternatively and/or additionally, the RNA interference comprises affecting expression of a marker gene selected from a group comprising or consisting of MBD3L2, TRIM43, PRAMEF1, ZSCAN4, KHDC1L, and LEUTX in a cell. In some aspects, the affecting expression of the marker gene is reducing expression of the marker gene at least 20%, at least 30%, at least 40%, at least 50%, at least 60% or more. In some aspects, the muscle dystrophy is Facioscapulohumeral muscular dystrophy (FSHD). Alternatively and/or additionally, the RNA interference comprises affecting expression of a marker gene selected from a group comprising or consisting of WFDC3, ILVBL, SLC15A2, and SORD in a cell. In some aspects, the affecting expression of the marker gene is reducing expression of the marker gene at least 20%, at least 30%, at least 40%, at least 50%, at least 60% or more. In some aspects, the muscle dystrophy is Facioscapulohumeral muscular dystrophy (FSHD).

In certain aspects, polynucleic acid molecule conjugate comprises a molecule of Formula (I): A-X-B, where A is the antibody or antigen binding fragment thereof, B is the polynucleic acid molecule that hybridizes to a target sequence of DUX4, X is a bond or a non-polymeric linker, which is conjugated to a cysteine residue of A.

Disclosed herein, in certain aspects, is a pharmaceutical composition comprising a polynucleic acid molecule conjugate as described herein, and a pharmaceutically acceptable excipient. In some aspects, the pharmaceutical composition is formulated as a nanoparticle formulation. In some aspects, the pharmaceutical composition is formulated for parenteral, oral, intranasal, buccal, rectal, transdermal, or intravenous, subcutaneous, or intrathecal administration.

The symptoms of FSHD include effects on skeletal muscles. The skeletal muscles affected by FSHD include muscles around the eyes and mouth, muscle of the shoulders, muscle of the upper arms, muscle of the lower legs, abdominal muscles and hip muscles. In some instances, the symptoms of FSHD also affects vision and hearing. In some instances, the symptoms of FSHD also affect the function of the heart or lungs. In some instances, the symptoms of FSHD include muscle weakness, muscle atrophy, muscle dystrophy, pain inflammation, contractures, scoliosis, lordosis, hypoventilation, abnormalities of the retina, exposure to keratitis, mild hearing loss, and EMG abnormality. The term muscle atrophy as used herein refers to a wide range of muscle related effects of FSHD.

Disclosed herein, in certain aspects, is a method for treating muscular dystrophy in a subject in need thereof by providing a polynucleic acid conjugate as described herein, and administering the polynucleic acid conjugate to the subject in needthereof to treat the muscular dystrophy. The polynucleic acid conjugate reduces a quantity of the mRNA transcript of human DUX4. In some aspects, the polynucleic acid moiety mediates RNA interference against the human DUX4 modulates muscle atrophy in a subject. In certain aspects, the RNA interference comprises affecting expression of a marker gene for DUX4 selected from a group comprising or consisting of MBD3L2, TRIM43, PRAMEF1, ZSCAN4, KHDC1L, and LEUTX in a cell affected by a muscle dystrophy. In certain aspects, the RNA interference comprises affecting expression of a marker gene for DUX4 selected from a group comprising or consisting of WFDC3, ILVBL, SLC15A2, and SORD in a cell affected by a muscle dystrophy.

Preferably, the muscular dystrophy is Facioscapulohumeral muscular dystrophy (FSHD).

Disclosed herein, in certain aspects, is a use of the polynucleic acid molecule conjugate or a pharmaceutical composition as described herein for treating in a subject diagnosed with or suspected to have Facioscapulohumeral muscular dystrophy (FSHD). Also disclosed herein, in certain aspects, is a use of the polynucleic acid molecule conjugate or the pharmaceutical composition as described herein for manufacturing a medicament for treating in a subject diagnosed with or suspected to have Facioscapulohumeral muscular dystrophy (FSHD).

Disclosed herein, in certain aspects, is a kit comprising the polynucleic acid molecule conjugate or the pharmaceutical composition as described herein.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIG. 1 illustrates a diagram of FSHD pathology.

FIG. 2 shows a flowchart diagram of in silico selection of DUX4 siRNA.

FIG. 3 illustrates the location and numbers of selected DUX4 siRNA in the DUX4 mRNA transcript.

FIG. 4A shows graphs of the in vivo downregulation of DUX4-target genes in skeletal muscles in mouse model of FSHD.

FIG. 4B shows graphs of the in vivo muscle tissue concentration of DUX-4 siRNA.

FIG. 5A illustrates a representative structure of siRNA with C₆—NH₂ conjugation handle at the 5′ end and C₆—SH at 3′end of the passenger strand or guide strand.

FIG. 5B illustrates a representative structure of siRNA passenger strand or guide strand with C₆—NH₂ conjugation handle at the 5′ end and C₆—S-PEG at 3′ end.

FIG. 5C illustrates a representative structure of siRNA passenger strand or guide strand with C₆—NH₂ conjugation handle at the 5′ end and C₆—S-NEM at 3′ end.

FIG. 5D illustrates a representative structure of siRNA passenger strand with C₆—N-SMCC conjugation handle at the 5′ end and C₆—S-NEM at 3′ end.

FIG. 5E illustrates a representative structure of siRNA passenger strand or guide strand with PEG at the 5′ end and C₆—SH at 3′ end.

FIG. 5F illustrates a representative structure of siRNA passenger strand or guide strand with C₆-S-NEM at the 5′ end and C₆—NH₂ conjugation handle at 3′ end.

FIG. 6A illustrates an antibody-Cys-SMCC-5′-passenger strand (Architecture-1). This conjugate was generated by antibody inter-chain cysteine conjugation to maleimide (SMCC) at the 5′ end of passenger strand.

FIG. 6B illustrates an antibody-Cys-SMCC-3′-Passenger strand (Architecture-2). This conjugate was generated by antibody inter-chain cysteine conjugation to maleimide (SMCC) at the 3′ end of passenger strand.

FIG. 6C illustrates an antibody-Cys-bisMal-3′-Passenger strand (ASC Architecture-3). This conjugate was generated by antibody inter-chain cysteine conjugation to bismaleimide (bisMal)linker at the 3′ end of passenger strand.

FIG. 6D illustrates a model structure of the Fab-Cys-bisMal-3′-Passenger strand (ASC Architecture-4). This conjugate was generated by Fab inter-chain cysteine conjugation to bismaleimide (bisMal) linker at the 3′ end of passenger strand.

FIG. 6E illustrates a model structure of the antibody siRNA conjugate with two different siRNAs attached to one antibody molecule (ASC Architecture-5). This conjugate was generated by conjugating a mixture of SSB and HPRT siRNAs to the reduced mAb inter-chain cysteines to bismaleimide (bisMal) linker at the 3′ end of passenger strand of each siRNA.

FIG. 6F illustrates a model structure of the antibody siRNA conjugate with two different siRNAs attached (ASC Architecture-6). This conjugate was generated by conjugating a mixture of SSB and HPRT siRNAs to the reduced mAb inter-chain cysteines to maleimide (SMCC) linker at the 3′ end of passenger strand of each siRNA.

FIG. 7A illustrates an exemplary synthesIs scheme (Synthesis scheme-1) for antibody-Cys-SMCC-siRNA-PEG conjugates via antibody cysteine conjugation.

FIG. 7B illustrates an exemplary synthesis scheme (Synthesis scheme-2) for antibody-Cys-BisMal-siRNA-PEG conjugates.

FIG. 7C illustrates an exemplary synthesis scheme (Synthesis scheme-3) for Fab-siRNA conjugate generation.

### DETAILED DESCRIPTION OF THE DISCLOSURE

FSHD is caused by aberrant expression of a gene, DUX4, in skeletal muscle resulting in the inappropriate presence of DUX4 protein. DUX4 itself is a transcription factor that induces the expression of other genes and it is these inappropriately expressed downstream genes that result in the muscle pathology. Normally DUX4-driven gene expression is limited to germline and early stem cell development. In patients with FSHD, the DUX4 protein in skeletal muscle regulates other gene products, some of which are toxic to the muscle. Evidence of aberrant DUX4-driven gene expression is the major molecular signature that distinguishes muscle tissue affected by FSHD from healthy muscle. The result of aberrant DUX4 expression in FSHD is death of muscle and its replacement by fat, resulting in skeletal muscle weakness and progressive disability. Data suggest that reducing expression of the DUX4 gene and its downstream transcriptional program could provide a disease-modifying therapeutic approach for the treatment of FSHD at its root cause.

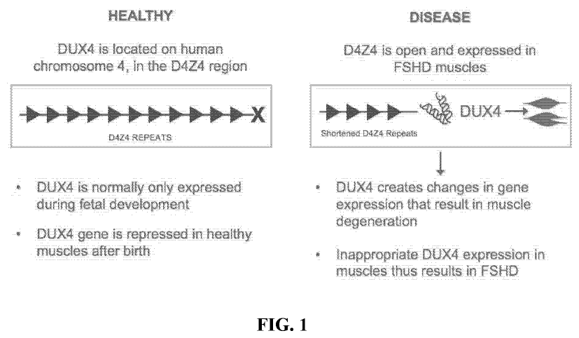

There are two ways the DUX4 gene can be unsilenced, or de-repressed. In FSHD1, which comprises approximately 95% of FSHD patients, there are mutations that lead to the shortening of an array of DNA in a region near the end of the long arm of chromosome 4, known as D4Z4, which has repeats in the subtelomeric region of the chromosome. The D4Z4 region is abnormally shortened and contains between 1-10 repeats instead of the normal 11 to 100 repeats. This contraction causes hypomethylation of the D4Z4 region and de-repression of DUX4. Patients with FSHD2 do not have a meaningful D4Z4 repeat contraction, but have mutations in a regulatory gene, known as the SMCHD1 gene, that normally contributes to the repression of the DUX4 gene via DNA methylation. When that repression is lost due to the mutations of the SMCHD1 gene leading to the hypomethylation of the D4Z4 region, DUX4 is inappropriately expressed, inducing the disease state. FIG. 1 shows an illustrative diagram of FSHD pathology.

Nucleic acid (e.g., RNAi) therapy is a targeted therapy with high selectivity and specificity. However, in some instances, nucleic acid therapy is also hindered by poor intracellular uptake, limited blood stability and non-specific immune stimulation. To address these issues, various modifications of the nucleic acid composition are explored, such as for example, novel linkers for better stabilizing and/or lower toxicity, optimization of binding moiety for increased target specificity and/or target delivery, and nucleic acid polymer modifications for increased stability and/or reduced off-target effect.

In some aspects, the arrangement or order of the different components that make-up the nucleic acid composition further effects intracellular uptake, stability, toxicity, efficacy, and/or non-specific immune stimulation. For example, if the nucleic acid component includes a binding moiety, a polymer, and a polynucleic acid molecule (or polynucleotide), the order or arrangement of the binding moiety, the polymer, and/or the polynucleic acid molecule (or polynucleotide) (e.g., binding moiety-polynucleic acid molecule-polymer, binding moiety-polymer-polynucleic acid molecule, or polymer-binding moiety-polynucleic acid molecule) further effects intracellular uptake, stability, toxicity, efficacy, and/or non-specific immune stimulation.

In some aspects, described herein include polynucleic acid molecules and polynucleic acid molecule conjugates for the treatment of Facioscapulohumeral Muscular Dystrophy (FSHD) especially muscle dystrophy and/or muscle atrophy associated therewith. In some instances, the polynucleic acid molecule conjugates described herein enhance intracellular uptake, stability, and/or efficacy. In some cases, the polynucleic acid molecule conjugates comprise an antibody or antigen binding fragment thereof conjugated to a polynucleic acid molecule. In some cases, the polynucleic acid molecules that hybridize to target sequences of DUX4, preferably human DUX4. In some cases, the nucleic acid molecules that hybridize to target sequences of human DUX4 having the accession number NM_001306068. In some cases, the nucleic acid molecules that hybridize to target sequences of human DUX4 having the SEQ ID NO: 439.

Additional aspects described herein include methods of treating FSHD, comprising administering to a subject a polynucleic acid molecule or a polynucleic acid molecule conjugate described herein.

### Polynucleic Acid Molecules

In certain aspects, a polynucleic acid molecule hybridizes to a target sequence of Double homeobox 4 (DUX4) gene. In some instances, a polynucleic acid molecule described herein hybridizes to a target sequence of human DUX4 gene (DUX4) and reduces DUX4 mRNA in muscle cells.

In some aspects, the polynucleic acid molecule comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-70. In some aspects, the polynucleic acid molecule comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 141-210. In some aspects, the polynucleic acid molecule comprises a sequence having at least 50%, 55%, 60, 65%, 70%, 75% 80%, 85%, 90%, 95%, 96%, 97%, 98%, 99%, or 100% sequence identity to a sequence selected from SEQ ID NOs: 71-140. In some aspects, the polynucleic acid molecule comprises a sequence having at least 50%, 55%, 60%, 6%5%, 70%, 75%, 80, 85, 90, 95% 96%, 97%, 98%, 99%, or 100% sequence identity to a sequence selected from SEQ ID NOs: 211-280.

In some aspects, the polynucleic acid molecule comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 142, 146, 196, 201-206, 412-420, or 430-438.

In some aspects, the polynucleic acid molecule comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-70. In some cases, the second polynucleotide comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 71-140. In some cases, the polynucleic acid molecule comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 141-210. In some cases, the second polynucleotide comprises a sequence having at least 50%, 55%, 60%, 65%, 70, 75%, 80%, 85, 90, 95% 96%, 97%, 98%, 99%, or 100% sequence identity to a sequence selected from SEQ ID NOs: 211-280.

In some instances, the first polynucleotide comprises a sequence having at least 50%, 55%, 60%, 65%, 70%, 75%, 80%, 85%, 90, 95% 96%, 97%, 98%, 99%, or 100% sequence identity to a sequence selected from SEQ ID NOs: 142, 146, 196, or 201-206. In some cases, the second polynucleotide comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 412-420 or 430-438.

In some aspects, the polynucleic acid molecule comprises a sense strand (e.g., a passenger strand) and an antisense strand (e.g., a guide strand). In some instances, the sense strand (e.g., the passenger strand) comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1-70. In some instances, the antisense strand (e.g., the guide strand) comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 71-140. In some aspects, the polynucleic acid molecule comprises a sense strand (e.g., a passenger strand) and an antisense strand (e.g., a guide strand). In some instances, the sense strand (e.g., the passenger strand) comprises a sequence having at least 50%, 55%, 60%, 65%, 70%, 75%, 80%, 85%, 90, 95% 96%, 97%, 98%, 99%, or 100% sequence identity to a sequence selected from SEQ ID NOs: 141-210. In some instances, the antisense strand (e.g., the guide strand) comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 211-280. In some instances, the sense strand (e.g., the passenger strand) comprises a sequence having at least 50%, 55%,60%,65%,70%,75%, 80%, 85%, 90%, 95%, 96%, 97%, 98%, 99%, or 100% sequence identity to a sequence selected from SEQ ID NOs: 142, 146, 196, or 201-206. In some instances, the antisense strand (e.g., the guide strand) comprises a sequence having at least 50%, 55% 60%, 65%, 70%, 75%, 80%, 85%, 90%, 95%, 96%, 97%, 98%, 99%, or 100% sequence identity to a sequence selected from SEQ ID NOs: 412-420 or 430-438.

In some instances, the sense strand comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 1, 2, 3, 6, 14, 36, 52, 56, 61, 62, 63, 65, or 66. In some instances, the antisense strand comprises a sequence having at least 50%, 55%, 60%, 65%, 70%, 75%, 80%, 85%, 90%, 95%, 96%, 97%, 98%, 99%, or 100% sequence identity to a sequence selected from SEQ ID NOs: 71, 72, 73, 76, 84, 106, 122, 127, 131, 132, 133, 135, or 136. In some instances, the siRNA comprises sense strand and antisense strand as presented in Table 11.

In some instances, the sense strand comprises a sequence having at least 50%, 55%, 60%, 65%, 70%, 75%, 80%, 85%, 90, 95% 96%, 97%, 98%, 99%, or 100% sequence identity to a sequence selected from SEQ ID NOs: 141, 142, 143, 146, 176, 192, 196, 201, 202, 203, 205, or 206. In some instances, the antisense strand comprises a sequence having at least 50%, 55%, 60%, 65%, 70%, 75%, 80%, 85%, 90, 95% 96%, 97%, 98%, 99%, or 100% sequence identity to a sequence selected from SEQ ID NOs: 211, 212, 213, 216, 246, 262, 266, 271, 272, 273, 275, or 276. In some instances, the siRNA comprises sense strand and antisense strand as presented in Table 12.

In some instances, the sense strand comprises a sequence having at least 50%, 55%, 60%, 65%, 70%, 75%, 80%, 85%, 90, 95% 96%, 97%, 98%, 99%, or 100% sequence identity to a sequence selected from SEQ ID NOs: 142, 146, 196, or 201-206 in Table 14 and Table 15.

In some instances, the antisense strand comprises a sequence having at least 50%, 55%, 60%, 6%5%, 70%, 75%, 80, 85, 90, 95% 96%, 97%, 98%, 99%, or 100% sequence identity to a sequence selected from SEQ ID NOs: 412-420 or 430-438 in Table 14 and Table 15.

In some embodiments, the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 412-420 or 430-438. In some embodiments, the polynucleic acid molecule comprises a sense strand and/or an antisense strand, and the antisense is identical to a sequence selected from SEQ ID NOs: 412-420 or 430-438. In some embodiments, the polynucleic acid molecule comprises a sense strand and/or an antisense strand, and the sense strand comprises at least 80%, at least 85%, at least 90%, at least 95%, at least 96%, at least 97%, at least 98%, at least 99% identical to a sequence selected from SEQ ID NOs: 142, 146, 196, or 201-206. In some embodiments, the polynucleic acid molecule comprises a sense strand and/or an antisense strand, and the sense strand is identical to a sequence selected from SEQ ID NOs: 142, 146, 196, or 201-206.

In some aspects, the sequence polynucleic acid molecule has at least 14, 15, 16, 17, 18, or 19 contiguous nucleotides differing by no more than 3 nucleotides, no more than 2 nucleotides, or no more than 1 nucleotide from any one of SEQ ID NOs: 142, 146, 196, or 201-206, or SEQ ID NOs: 412-420 or 430-438. In some aspects, the polynucleic acid molecule is single-stranded. In some aspects, the polynucleic acid molecule is double-stranded.

In some aspects, the polynucleic acid molecule described herein comprises RNA or DNA. In some cases, the polynucleic acid molecule comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the oligonucleotide is a phosphorodiamidate morpholino oligomers (PMO), which are short single-stranded oligonucleotide analogs that are built upon a backbone of morpholine rings connected by phosphorodiamidate linkages. In some instances, the RNA comprises siRNA. In some instances, the polynucleic acid molecule comprises siRNA.

In some aspects, the polynucleic acid molecule is from about 8 to about 50 nucleotides in length. In some aspects, the polynucleic acid molecule is from about 10 to about 50 nucleotides in length. In some instances, the polynucleic acid molecule is from about 10 to about 30, from about 15 to about 30, from about 18 to about 25, form about 18 to about 24, from about 19 to about 23, or from about 20 to about 22 nucleotides in length.

In some aspects, the polynucleic acid molecule is about 50 nucleotides in length. In some instances, the polynucleic acid molecule is about 45 nucleotides in length. In some instances, the polynucleic acid molecule is about 40 nucleotides in length. In some instances, the polynucleic acid molecule is about 35 nucleotides in length. In some instances, the polynucleic acid molecule is about 30 nucleotides in length. In some instances, the polynucleic acid molecule is about 25 nucleotides in length. In some instances, the polynucleic acid molecule is about 20 nucleotides in length. In some instances, the polynucleic acid molecule is about 19 nucleotides in length. In some instances, the polynucleic acid molecule is about 18 nucleotides in length. In some instances, the polynucleic acid molecule is about 17 nucleotides in length. In some instances, the polynucleic acid molecule is about 16 nucleotides in length. In some instances, the polynucleic acid molecule is about 15 nucleotides in length. In some instances, the polynucleic acid molecule is about 14 nucleotides in length. In some instances, the polynucleic acid molecule is about 13 nucleotides in length. In some instances, the polynucleic acid molecule is about 12 nucleotides in length. In some instances, the polynucleic acid molecule is about 11 nucleotides in length. In some instances, the polynucleic acid molecule is about 10 nucleotides in length. In some instances, the polynucleic acid molecule is about 8 nucleotides in length. In some instances, the polynucleic acid molecule is between about 8 and about 50 nucleotides in length. In some instances, the polynucleic acid molecule is between about 10 and about 50 nucleotides in length. In some instances, the polynucleic acid molecule is between about 10 and about 45 nucleotides in length. In some instances, the polynucleic acid molecule is between about 10 and about 40 nucleotides in length. In some instances, the polynucleic acid molecule is between about 10 and about 35 nucleotides in length. In some instances, the polynucleic acid molecule is between about 10 and about 30 nucleotides in length. In some instances, the polynucleic acid molecule is between about 10 and about 25 nucleotides in length. In some instances, the polynucleic acid molecule is between about 10 and about 20 nucleotides in length.

In some instances, the polynucleic acid molecule is between about 15 and about 25 nucleotides in length. In some instances, the polynucleic acid molecule is between about 15 and about 30 nucleotides in length. In some instances, the polynucleic acid molecule is between about 12 and about 30 nucleotides in length.

In some aspects, the polynucleic acid molecule comprises a first polynucleotide. In some instances, the polynucleic acid molecule comprises a second polynucleotide. In some instances, the polynucleic acid molecule comprises a first polynucleotide and a second polynucleotide. In some instances, the first polynucleotide is a sense strand or passenger strand. In some instances, the second polynucleotide is an antisense strand or guide strand.

In some aspects, the polynucleic acid molecule is a first polynucleotide. In some aspects, the first polynucleotide is from about 8 to about 50 nucleotides in length. In some aspects, the first polynucleotide is from about 10 to about 50 nucleotides in length. In some instances, the first polynucleotide is from about 10 to about 30, from about 15 to about 30, from about 18 to about 25, form about 18 to about 24, from about 19 to about 23, or from about 20 to about 22 nucleotides in length.

In some instances, the first polynucleotide is about 50 nucleotides in length. In some instances, the first polynucleotide is about 45 nucleotides in length. In some instances, the first polynucleotide is about 40 nucleotides in length. In some instances, the first polynucleotide is about 35 nucleotides in length. In some instances, the first polynucleotide is about 30 nucleotides in length. In some instances, the first polynucleotide is about 25 nucleotides in length. In some instances, the first polynucleotide is about 20 nucleotides in length. In some instances, the first polynucleotide is about 19 nucleotides in length. In some instances, the first polynucleotide is about 18 nucleotides in length. In some instances, the first polynucleotide is about 17 nucleotides in length. In some instances, the first polynucleotide is about 16 nucleotides in length. In some instances, the first polynucleotide is about 15 nucleotides in length. In some instances, the first polynucleotide is about 14 nucleotides in length. In some instances, the first polynucleotide is about 13 nucleotides in length. In some instances, the first polynucleotide is about 12 nucleotides in length. In some instances, the first polynucleotide is about 11 nucleotides in length. In some instances, the first polynucleotide is about 10 nucleotides in length. In some instances, the first polynucleotide is about 8 nucleotides in length. In some instances, the first polynucleotide is between about 8 and about 50 nucleotides in length. In some instances, the first polynucleotide is between about 10 and about 50 nucleotides in length. In some instances, the first polynucleotide is between about 10 and about 45 nucleotides in length. In some instances, the first polynucleotide is between about 10 and about 40 nucleotides in length. In some instances, the first polynucleotide is between about 10 and about 35 nucleotides in length. In some instances, the first polynucleotide is between about 10 and about 30 nucleotides in length. In some instances, the first polynucleotide is between about 10 and about 25 nucleotides in length. In some instances, the first polynucleotide is between about 10 and about 20 nucleotides in length. In some instances, the first polynucleotide is between about 15 and about 25 nucleotides in length. In some instances, the first polynucleotide is between about 15 and about 30 nucleotides in length. In some instances, the first polynucleotide is between about 12 and about 30 nucleotides in length.

In some aspects, the polynucleic acid molecule is a second polynucleotide. In some aspects, the second polynucleotide is from about 8 to about 50 nucleotides in length. In some aspects, the second polynucleotide is from about 10 to about 50 nucleotides in length. In some instances, the second polynucleotide is from about 10 to about 30, from about 15 to about 30, from about 18 to about 25, form about 18 to about 24, from about 19 to about 23, or from about 20 to about 22 nucleotides in length.

In some instances, the second polynucleotide is about 50 nucleotides in length. In some instances, the second polynucleotide is about 45 nucleotides in length. In some instances, the second polynucleotide is about 40 nucleotides in length. In some instances, the second polynucleotide is about 35 nucleotides in length. In some instances, the second polynucleotide is about 30 nucleotides in length. In some instances, the second polynucleotide is about 25 nucleotides in length. In some instances, the second polynucleotide is about 20 nucleotides in length. In some instances, the second polynucleotide is about 19 nucleotides in length. In some instances, the second polynucleotide is about 18 nucleotides in length. In some instances, the second polynucleotide is about 17 nucleotides in length. In some instances, the second polynucleotide is about 16 nucleotides in length. In some instances, the second polynucleotide is about 15 nucleotides in length. In some instances, the second polynucleotide is about 14 nucleotides in length. In some instances, the second polynucleotide is about 13 nucleotides in length. In some instances, the second polynucleotide is about 12 nucleotides in length. In some instances, the second polynucleotide is about 11 nucleotides in length. In some instances, the second polynucleotide is about 10 nucleotides in length. In some instances, the second polynucleotide is about 8 nucleotides in length. In some instances, the second polynucleotide is between about 8 and about 50 nucleotides in length. In some instances, the second polynucleotide is between about 10 and about 50 nucleotides in length. In some instances, the second polynucleotide is between about 10 and about 45 nucleotides in length. In some instances, the second polynucleotide is between about 10 and about 40 nucleotides in length. In some instances, the second polynucleotide is between about 10 and about 35 nucleotides in length. In some instances, the second polynucleotide is between about 10 and about 30 nucleotides in length. In some instances, the second polynucleotide is between about 10 and about 25 nucleotides in length. In some instances, the second polynucleotide is between about 10 and about 20 nucleotides in length. In some instances, the second polynucleotide is between about 15 and about 25 nucleotides in length. In some instances, the second polynucleotide is between about 15 and about 30 nucleotides in length. In some instances, the second polynucleotide is between about 12 and about 30 nucleotides in length.

In some aspects, the polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides. In some aspects, the polynucleic acid molecule comprises a sense strand and an antisense strand, and the antisense strand includes two non-base pairing nucleotides as an overhang at the 3′-end while the sense strand has no overhang. Optionally, in such aspects, the non-base pairing nucleotides have a sequence of TT, dTdT, or UU. In some aspects, the polynucleic acid molecule comprises a sense strand and an antisense strand, and the sense strand has one or more nucleotides at the 5′-end that are complementary to the antisense sequence.

In some aspects, the sequence of the polynucleic acid molecule is at least 40%, 50%, 55%, 60, 65, 70%, 75%, 80, 85%, 90%, 95%, 98%, 99%, or 99.5% complementary to a target sequence of DUX4. In some aspects, the target sequence of DUX4 is a nucleic acid sequence of about 10-50 base pair length, about 15-50 base pair length, 15-40 base pair length, 15-30 base pair length, or 15-25 base pair length sequences in DUX4, in which the first nucleotide of the target sequence starts at any nucleotide in DUX4 mRNA transcript in the coding region, or in the 5′ or 3′-untranslated region (UTR). For example, the first nucleotide of the target sequence can be selected so that it starts at the nucleic acid location (nal, number starting from the 5′-end of the full length of DUX mRNA, e.g., the 5′-end first nucleotide is nal.1) 1,nal2,nal3,nal4,nal5,nal6,nal7,nal8, nal9, nal 10, nal 11, nal 12, nal 13, nal 14, nal 15, nal 15, nal 16, nal 17, or any other nucleic acid location in the coding or noncoding regions (5′ or 3′-untraslated region) of DUX mRNA. In some aspects, the first nucleotide of the target sequence can be selected so that it starts at a location within, or between, nal 10-nal 15, nal10-nal20, nal50-nal60, nal55-nal65, nal75-nal 85, na195-nal 105, nal 135-nal 145, nal 155-nal 165, nal225-nal235, nal265-nal275, nal275-nal285, nal285-nal295, nal 325-nal335, nal335-nal345, nal385-nal395, nal515-nal525, nal665-nal675, nal675-nal685, nal 695-nal 705, nal 705-nal 715, nal 875-nal 885, nal 885-nal 895, nal 895-nal 905, nal 1035-nal1045, na 1045-nal1055, nal1125-nal1135, nal1135-nal1145, nal1145-nal 1155, nal1155-nal1165, nal1125-nal1135,nal1155-nal 1165, nal 1225-nal 1235,nal 1235-nal 1245,nal1275-nal 1285,nal 1285-nal 1295, nal 1305-nal 1315, nal1125-nal1135,nal 1155-nal 1165, nal 1225-nal 1235, nal 1235-nal 1245, nal 1275-nal 1285, nal 1285-nal 1295, nal 1305-nal1315,nal 1315-nal1325,nal1335-nal1345,nal1345-nal1355,nal1525-nal 1535, nal 1535-nal 1545, nal 1605-nal 1615, nal 1615-c.1625, nal 1625-nal 1635.

In some aspects, the sequence of the polynucleic acid molecule is at least 50% complementary to a target sequence described herein. In some aspects, the sequence of the polynucleic acid molecule is at least 60% complementary to a target sequence described herein. In some aspects, the sequence of the polynucleic acid molecule is at least 70% complementary to a target sequence described herein. In some aspects, the sequence of the polynucleic acid molecule is at least 80% complementary to a target sequence described herein. In some aspects, the sequence of the polynucleic acid molecule is at least 90% complementary to a target sequence described herein. In some aspects, the sequence of the polynucleic acid molecule is at least 95% complementary to a target sequence described herein. In some aspects, the sequence of the polynucleic acid molecule is at least 99% complementary to a target sequence described herein. In some instances, the sequence of the polynucleic acid molecule is 100% complementary to a target sequence described herein.

In some aspects, the sequence of the polynucleic acid molecule has five or fewer mismatches to a target sequence described herein. In some aspects, the sequence of the polynucleic acid molecule has four or fewer mismatches to a target sequence described herein. In some instances, the sequence of the polynucleic acid molecule has three or fewer mismatches to a target sequence described herein. In some cases, the sequence of the polynucleic acid molecule has two or fewer mismatches to a target sequence described herein. In some cases, the sequence of the polynucleic acid molecule has one or fewer mismatches to a target sequence described herein.

In some aspects, a group of polynucleic acid molecules among all the polynucleic acid molecules potentially binds to the target sequence of DUX4 are selected to generate a polynucleic acid molecule library. In certain aspects, such selection process is conducted in silico via one or more steps of eliminating less desirable polynucleic acid molecules from candidates. For example, in some aspects, the selection process comprises an elimination step of one or more polynucleic acid molecule that has single nucleotide polymorphism (SNP) and/or MEF<−5. Alternatively and/or additionally, in some aspects, the selection process comprises an elimination step of one or more polynucleic acid molecule with 0 and 1 mismatch (MM) in the human transcriptome (such that only hits allowed are DUX, DUX5, and DBET). Alternatively and/or additionally, in some aspects, the selection process comprises an elimination step of one or more polynucleic acid molecule with 0 MM in the human intragenic regions (such that only hits allowed are DUX1, DUX5 and DBET pseudogenes). Alternatively and/or additionally, in some aspects, the selection process comprises an elimination step of one or more polynucleic acid molecule with a MM to DUX4 human sequence used in FLExDUX4 FSHD mouse model.

Alternatively and/or additionally, in some aspects, the selection process comprises an elimination step of one or more polynucleic acid molecule predicted viability<60. Alternatively and/or additionally, such selection process comprises carrying forward one or more polynucleic acid molecule predicted viability>60. Alternatively and/or additionally, in some aspects, the selection process comprises an elimination step of one or more polynucleic acid molecule with a match to a seed region of known miRNAs 1-1000. Alternatively and/or additionally, in some aspects, the selection process comprises an elimination step of one or more polynucleic acid molecule with % GC content 75 and above. Alternatively and/or additionally, in some aspects, the selection process comprises a selection step of eight or fewer predicted off-target hits with 2 MM. In some aspects, for the region 295-1132 (nal 295-1132), 12 or fewer predicted off-target hits with 2 MM is allowed.

In some aspects, selection process is conducted in silico via one or more consecutive steps of eliminating less desirable polynucleic acid molecules from candidates. For example, in some aspects, selection process begins with collecting candidate polynucleic acid molecules to generate a library. From the library, the first eliminating step comprises eliminating one or more polynucleic acid molecule that has single nucleotide polymorphism (SNP) and/or MEF<−5. Then, the second eliminating step comprises eliminating one or more polynucleic acid molecule with 0 and 1 MM in the human transcriptome (such that only hits allowed are DUX, DUX5, and DBET). Then, the third eliminating step comprises eliminating one or more polynucleic acid molecule with 0 MM in the human intragenic regions (such that only hits allowed are DUX1, DUX5 and DBET pseudogenes). Then, the next eliminating step comprises eliminating one or more polynucleic acid molecule with a MM to DUX4 human sequence used in FLExDUX4 FSHD mouse model. Then, the next step is carrying forward only or one or more polynucleic acid molecule with predicted viability>60. Next, the eliminating step comprises eliminating one or more polynucleic acid molecule with a match to a seed region of known miRNAs 1-1000. Then, the eliminating step continues with eliminating one or more polynucleic acid molecule with % GC content 75 and above. Then, the final selection process comprises with eight or fewer predicted off-target hits with 2 MM, except for the region 295-1132, for which up to 12 hits are allowed.

In some aspects, the specificity of the polynucleic acid molecule that hybridizes to a target sequence described herein is a 95%, 98%, 99%, 99.5%, or 100% sequence complementarity of the polynucleic acid molecule to a target sequence. In some instances, the hybridization is a high stringent hybridization condition.

In some aspects, the polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

In some aspects, the polynucleic acid molecule comprises natural or synthetic or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some aspects, nucleotide analogues or artificial nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, and disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′-O-methoxyethyl modification adds a methoxyethyl group to the 2′ hydroxyl group of the ribose moiety. Exemplary chemical structures of a 2′-O-methyl modification of an adenosine molecule and 2′-O-methoxyethyl modification of an uridine are illustrated below.

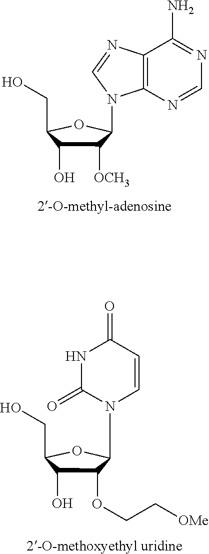

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

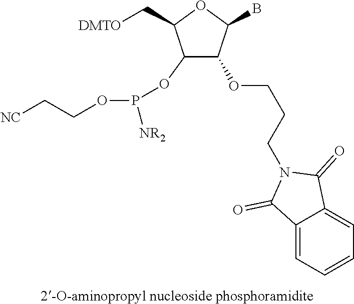

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (³E) conformation of the furanose ring of an LNA monomer.

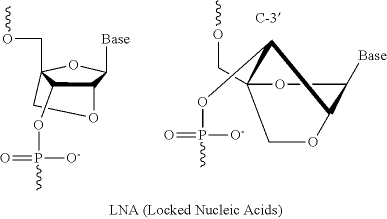

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA. Exemplary chemical structures of the ENA and bridged nucleic acids are illustrated below.

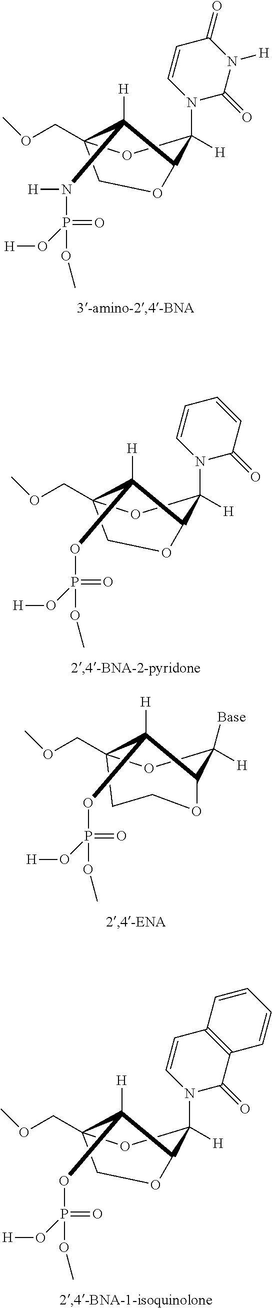

In some aspects, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, nucleotide analogues comprise modified bases such as, but not limited to, 5-propynyluridine, 5-propynylcytidine, 6-methyladenine, 6-methylguanine, N, N, -dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza- adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O-and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2, 4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′- anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five member ribose ring is substituted with a six member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

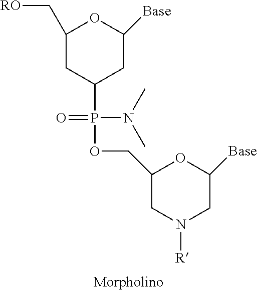

In some aspects, peptide nucleic acid (PNA) does not contain sugar ring or phosphate linkage and the bases are attached and appropriately spaced by oligoglycine-like molecules, therefore, eliminating a backbone charge.

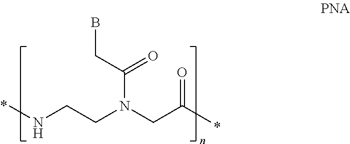

In some aspects, one or more modifications optionally occur at the internucleotide linkage. In some instances, modified internucleotide linkage include, but is not limited to, phosphorothioates, phosphorodithioates, methylphosphonates, 5′- alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′- alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisense oligonucleotides (PS ASO) are antisense oligonucleotides comprising a phosphorothioate linkage. An exemplary PS ASO is illustrated below.

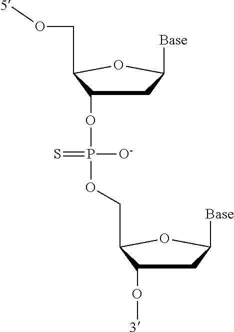

In some instances, the modification is a methyl or thiol modification such as methylphosphonate or thiolphosphonate modification. Exemplary thiolphosphonate nucleotide (left) and methylphosphonate nucleotide (right) are illustrated below.

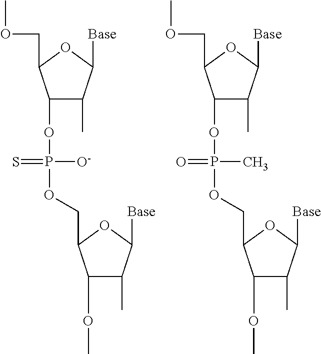

In some instances, a modified nucleotide includes, but is not limited to, 2′-fluoro N3-P5′-phosphoramidites illustrated as:

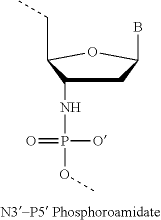

In some instances, a modified nucleotide includes, but is not limited to a 5′-vinyiphosphonate modified non-natural nucleotide selected from:

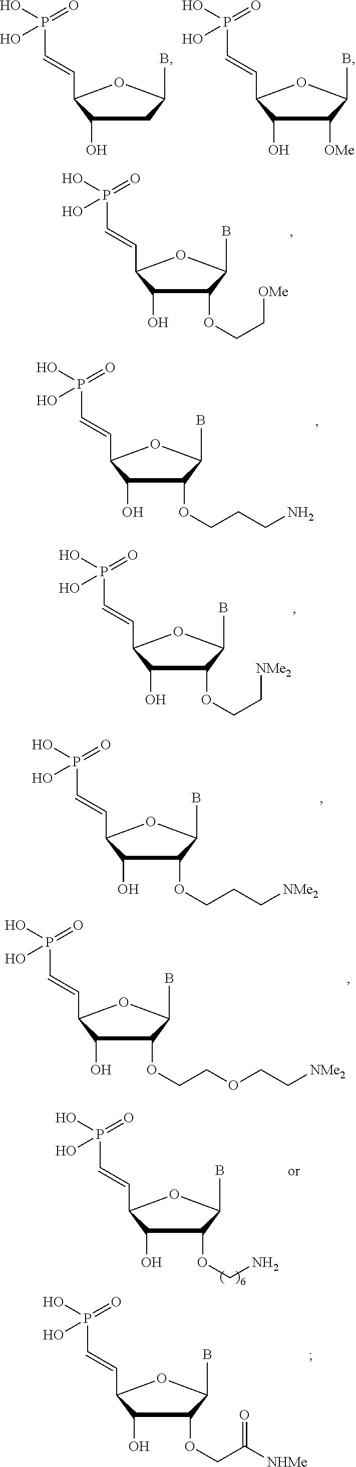

where B is a heterocyclic base moiety.

In some instances, a modified nucleotide includes, but is not limited to one 5′-vinyiphosphonate modified non-natural nucleotide selected from:

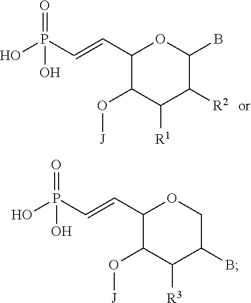

where B is a heterocyclic base moiety; R1, R2, and R3 are independently selected from hydrogen, halogen, alkyl or alkoxy; and J is an internucleotide linking group linking to the adjacent nucleotide of the polynucleotide.

In some instances, a modified nucleotide includes, but is not limited to one 5′-vinyiphosphonate modified non-natural nucleotide selected from:

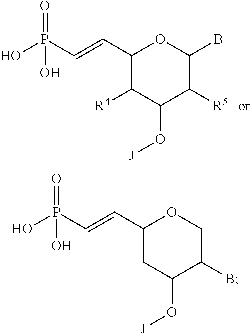

where B is a heterocyclic base moiety; R4, and R5 are independently selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from:

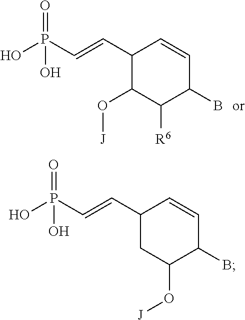

where B is a heterocyclic base moiety; R6 is selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide selected from locked nucleic acid (LNA) or ethylene nucleic acid (ENA).

In some instances, a modified nucleotide includes, but is not limited to one 5′-vinylphosphonate modified non-natural nucleotide selected from:

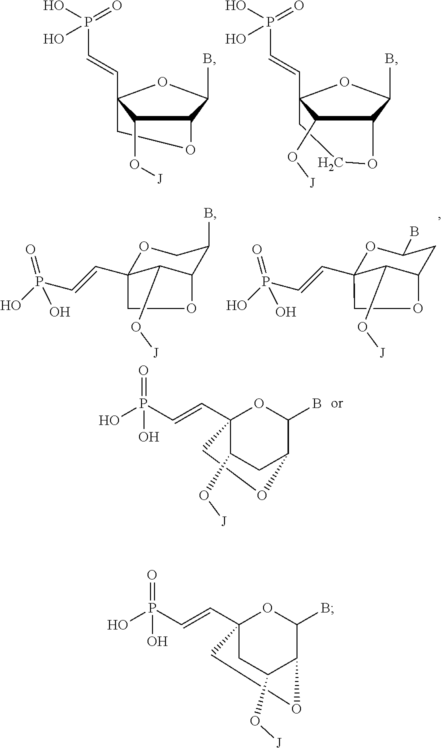

where B is a heterocyclic base moiety; and J is an internucleotide linking group linking to the adjacent nucleotide of the polynucleotide.

In some instances, a modified nucleotide includes, but is not limited to one 5′-vinylphosphonate modified non-natural nucleotide selected from:

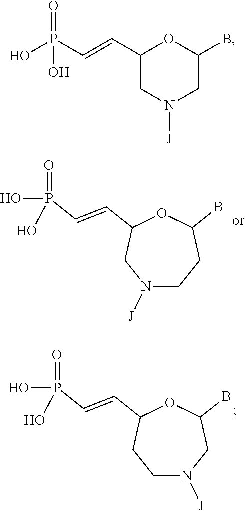

where B is a heterocyclic base moiety; and J is an internucleotide linking group linking to the adjacent nucleotide of the polynucleotide.

In some instances, a modified nucleotide includes, but is not limited to one 5′-vinylphosphonate modified non-natural nucleotide selected from:

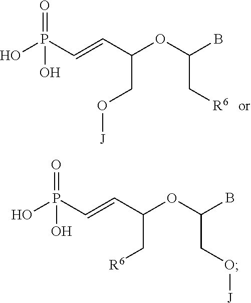

where B is a heterocyclic base moiety; R6 is selected from hydrogen, halogen, alkyl or alkoxy; and J is an internucleotide linking group linking to the adjacent nucleotide of the polynucleotide.

In some instances, a modified nucleotide includes, but is not limited to one 5′-vinylphosphonate modified non-natural nucleotide is:

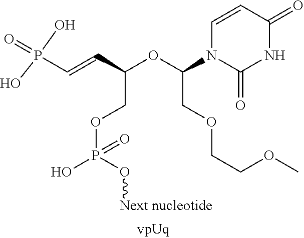

In some instances, a modified nucleotide includes, but is not limited to, hexitol nucleic acid (or 1′, 5′- anhydrohexitol nucleic acids (HNA)) illustrated as:

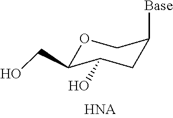

In some aspects, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with amnino alkyl group, e.g., a 3 C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some aspects, the polynucleic acid molecule comprises one or more of the artificial nucleotide analogues described herein. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoroN3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20,25, or more of2′-O-methyl modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some cases, the polynucleic acid molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification.

In some cases, the polynucleic acid molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification.

In some instances, the polynucleic acid molecule comprises at least one of: from about 10% to about 70% modification, from about 20% to about 70% modification, from about 30% to about 70% modification, from about 40% to about 70% modification, from about 50% to about 70% modification, and from about 60% to about 70% modification.

In some instances, the polynucleic acid molecule comprises at least one of: from about 10% to about 60% modification, from about 20% to about 60% modification, from about 30% to about 60% modification, from about 40% to about 60% modification, and from about 50% to about 60% modification.

In some cases, the polynucleic acid molecule comprises at least one of: from about 10% to about 50% modification, from about 20% to about 50% modification, from about 30% to about 50% modification, and from about 40% to about 50% modification.

In some cases, the polynucleic acid molecule comprises at least one of: from about 10% to about 40% modification, from about 20% to about 40% modification, and from about 30% to about 40% modification.

In some cases, the polynucleic acid molecule comprises at least one of: from about 10% to about 30% modification, and from about 20% to about 30% modification.

In some cases, the polynucleic acid molecule comprises from about 10% to about 20% modification.

In some cases, the polynucleic acid molecule comprises from about 15% to about 90%, from about 20% to about 80%, from about 30% to about 70%, or from about 40% to about 60% modifications.

In additional cases, the polynucleic acid molecule comprises at least about 15%, 20%, 30%, 40%, 50%, 60%, 70%, 80%, 90%, 95%, or 99% modification.

In some aspects, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modifications.

In some instances, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides.

In some instances, from about 5 to about 100% of the polynucleic acid molecule comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the polynucleic acid molecule comprise the artificial nucleotide analogues described herein. In some instances, about 5% of the polynucleic acid molecule comprises the artificial nucleotide analogues described herein. In some instances, about 10% of the polynucleic acid molecule comprises the artificial nucleotide analogues described herein. In some instances, about 15% of the polynucleic acid molecule comprises the artificial nucleotide analogues described herein. In some instances, about 20% of the polynucleic acid molecule comprises the artificial nucleotide analogues described herein. In some instances, about 25% of the polynucleic acid molecule comprises the artificial nucleotide analogues described herein. In some instances, about 30% of the polynucleic acid molecule comprises the artificial nucleotide analogues described herein. In some instances, about 35% of the polynucleic acid molecule comprises the artificial nucleotide analogues described herein. In some instances, about 40% of the polynucleic acid molecule comprises the artificial nucleotide analogues described herein. In some instances, about 45% of the polynucleic acid molecule comprises the artificial nucleotide analogues described herein. In some instances, about 50% of the polynucleic acid molecule comprises the artificial nucleotide analogues described herein. In some instances, about 55% of the polynucleic acid molecule comprises the artificial nucleotide analogues described herein. In some instances, about 60% of the polynucleic acid molecule comprises the artificial nucleotide analogues described herein. In some instances, about 65% of the polynucleic acid molecule comprises the artificial nucleotide analogues described herein. In some instances, about 70% of the polynucleic acid molecule comprises the artificial nucleotide analogues described herein. In some instances, about 75% of the polynucleic acid molecule comprises the artificial nucleotide analogues described herein. In some instances, about 80% of the polynucleic acid molecule comprises the artificial nucleotide analogues described herein. In some instances, about 85% of the polynucleic acid molecule comprises the artificial nucleotide analogues described herein. In some instances, about 90% of the polynucleic acid molecule comprises the artificial nucleotide analogues described herein. In some instances, about 95% of the polynucleic acid molecule comprises the artificial nucleotide analogues described herein. In some instances, about 96% of the polynucleic acid molecule comprises the artificial nucleotide analogues described herein. In some instances, about 97% of the polynucleic acid molecule comprises the artificial nucleotide analogues described herein. In some instances, about 98% of the polynucleic acid molecule comprises the artificial nucleotide analogues described herein. In some instances, about 99% of the polynucleic acid molecule comprises the artificial nucleotide analogues described herein. In some instances, about 100% of the polynucleic acid molecule comprises the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some aspects, the polynucleic acid molecule comprises from about one to about 25 modifications in which the modification comprises an artificial nucleotide analogues described herein. In some aspects, the polynucleic acid molecule comprises about one modification in which the modification comprises an artificial nucleotide analogue described herein. In some aspects, the polynucleic acid molecule comprises about two modifications in which the modifications comprise an artificial nucleotide analogue described herein. In some aspects, the polynucleic acid molecule comprises about three modifications in which the modifications comprise an artificial nucleotide analogue described herein. In some aspects, the polynucleic acid molecule comprises about four modifications in which the modifications comprise an artificial nucleotide analogue described herein. In some aspects, the polynucleic acid molecule comprises about five modifications in which the modifications comprise an artificial nucleotide analogue described herein. In some aspects, the polynucleic acid molecule comprises about six modifications in which the modifications comprise an artificial nucleotide analogue described herein. In some aspects, the polynucleic acid molecule comprises about seven modifications in which the modifications comprise an artificial nucleotide analogue described herein. In some aspects, the polynucleic acid molecule comprises about eight modifications in which the modifications comprise an artificial nucleotide analogue described herein. In some aspects, the polynucleic acid molecule comprises about nine modifications in which the modifications comprise an artificial nucleotide analogue described herein. In some aspects, the polynucleic acid molecule comprises about 10 modifications in which the modifications comprise an artificial nucleotide analogue described herein. In some aspects, the polynucleic acid molecule comprises about 11 modifications in which the modifications comprise an artificial nucleotide analogue described herein. In some aspects, the polynucleic acid molecule comprises about 12 modifications in which the modifications comprise an artificial nucleotide analogue described herein. In some aspects, the polynucleic acid molecule comprises about 13 modifications in which the modifications comprise an artificial nucleotide analogue described herein. In some aspects, the polynucleic acid molecule comprises about 14 modifications in which the modifications comprise an artificial nucleotide analogue described herein. In some aspects, the polynucleic acid molecule comprises about 15 modifications in which the modifications comprise an artificial nucleotide analogue described herein. In some aspects, the polynucleic acid molecule comprises about 16 modifications in which the modifications comprise an artificial nucleotide analogue described herein. In some aspects, the polynucleic acid molecule comprises about 17 modifications in which the modifications comprise an artificial nucleotide analogue described herein. In some aspects, the polynucleic acid molecule comprises about 18 modifications in which the modifications comprise an artificial nucleotide analogue described herein. In some aspects, the polynucleic acid molecule comprises about 19 modifications in which the modifications comprise an artificial nucleotide analogue described herein. In some aspects, the polynucleic acid molecule comprises about 20 modifications in which the modifications comprise an artificial nucleotide analogue described herein. In some aspects, the polynucleic acid molecule comprises about 21 modifications in which the modifications comprise an artificial nucleotide analogue described herein. In some aspects, the polynucleic acid molecule comprises about 22 modifications in which the modifications comprise an artificial nucleotide analogue described herein. In some aspects, the polynucleic acid molecule comprises about 23 modifications in which the modifications comprise an artificial nucleotide analogue described herein. In some aspects, the polynucleic acid molecule comprises about 24 modifications in which the modifications comprise an artificial nucleotide analogue described herein. In some aspects, the polynucleic acid molecule comprises about 25 modifications in which the modifications comprise an artificial nucleotide analogue described herein.

In some aspects, a polynucleic acid molecule is assembled from two separate polynucleotides wherein one polynucleotide comprises the sense strand and the second polynucleotide comprises the antisense strand of the polynucleic acid molecule. In other aspects, the sense strand is connected to the antisense strand via a linker molecule, which in some instances is a polynucleotide linker or a non-nucleotide linker.

In some aspects, a polynucleic acid molecule comprises a sense strand and an antisense strand, wherein pyrimidine nucleotides in the sense strand comprises 2′-O-methylpyrimidine nucleotides and purine nucleotides in the sense strand comprise 2′-deoxy purine nucleotides. In some aspects, a polynucleic acid molecule comprises a sense strand and an antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some aspects, a polynucleic acid molecule comprises a sense strand and an antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some aspects, a polynucleic acid molecule comprises a sense strand and an antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some aspects, a polynucleic acid molecule comprises a sense strand and an antisense strand, and at least one of sense strand and an antisense strand has a plurality of (e.g., two or more, three or more, four or more, five or more, six or more, seven or more, eight or more, etc.) 2′-O-methyl or 2′-deoxy-2′-fluoro modified nucleotides. In some aspects, where at least two out of the a plurality of 2′-O-methyl or 2′-deoxy-2′-fluoro modified nucleotides are consecutive nucleotides. In some aspects, where consecutive 2′-O-methyl or 2′-deoxy-2′-fluoro modified nucleotides are located at the 5′-end of the sense strand and/or the antisense strand. In some aspects, where consecutive 2′-O-methyl or 2′-deoxy-2′-fluoro modified nucleotides are located at the 3′-end of the sense strand and/or the antisense strand. In some aspects, the sense strand of polynucleic acid molecule includes at least four, at least five, at least six consecutive 2′-O-methyl modified nucleotides at its 5′ end and/or 3′end, or both. Optionally, in such aspects, the sense strand of polynucleic acid molecule includes at least one, at least two, at least three, at least four 2′-deoxy-2′-fluoro modified nucleotides at the 3′ end of the at least four, at least five, at least six consecutive 2′-O-methyl modified nucleotides at the polynucleotides' 5′ end, or at the 5′ end of the at least four, at least five, at least six consecutive 2′-O-methyl modified nucleotides at polynucleotides' 3′ end. Also optionally, such at least two, at least three, at least four 2′-deoxy-2′-fluoro modified nucleotides are consecutive nucleotides.

In some aspects, a polynucleic acid molecule comprises a sense strand and an antisense strand, and at least one of sense strand and antisense strands has 2′-O-methyl modified nucleotide located at the 5′-end of the sense strand and/or the antisense strand. In some aspects, at least one of sense strand and antisense strands has 2′-O-methyl modified nucleotide located at the 3′-end of the sense strand and/or the antisense strand. In some aspects, the 2′-O-methyl modified nucleotide located at the 5′-end of the sense strand and/or the antisense strand is a purine nucleotide. In some aspects, the 2′-O-methyl modified nucleotide located at the 5′-end of the sense strand and/or the antisense strand is a pyridine nucleotide.

In some aspects, a polynucleic acid molecule comprises a sense strand and an antisense strand, and the antisense strand has two or more consecutive 2′-deoxy-2′-fluoro modified nucleotides at 5′-end. In some aspects, a polynucleic acid molecule comprises a sense strand and an antisense strand, and the antisense strand has two or more consecutive 2′-O-methyl modified nucleotides at 3′-end. In some aspects, a polynucleic acid molecule comprises a sense strand and an antisense strand, and the antisense strand has at least 2, 3, 4, 5, 6, or 7 consecutive 2′-O-methyl modified nucleotides.

In some aspects, a polynucleic acid molecule comprises a sense strand and an antisense strand, and the sense strand comprises a nucleic acid of 5′-nsnsnnnnNfNfNfnnnnnnnnsnsa-3′ (lower case (n)=2′-O-Me (methyl), Nf=2′-F (fluoro); s=phosphorothioate backbone modification). In some aspects, a polynucleic acid molecule comprises a sense strand and an antisense strand, and the antisense strand comprises a nucleic acid of 5′-UfsNfsnnnNfnnnnnnnNfnNfnnnsusu-3′ (lower case (n)=2′-O-Me (methyl), Nf=2′-F (fluoro); s=phosphorothioate backbone modification). In some aspects, a polynucleic acid molecule comprises a sense strand and an antisense strand, and the sense strand comprises a nucleic acid of 5′-nsnsnnnnNfNfNfnnnnnnnnsnsa-3′ (lower case (n)=2′-O-Me (methyl), Nf=2′-F (fluoro); s=phosphorothioate backbone modification) and the antisense strand comprises a nucleic acid of 5′-UfsNfsnnnNfnnnnnnnNfnNfnnnsusu-3′ (lower case (n)=2′-O-Me (methyl), Nf=2′-F (fluoro); s=phosphorothioate backbone modification).

In some aspects, a polynucleic acid molecule comprises a sense strand and an antisense strand, wherein the sense strand includes a terminal cap moiety at the 5′-end, the 3′-end, or both of the 5′ and 3′ ends of the sense strand. In other aspects, the terminal cap moiety is an inverted deoxy abasic moiety.

In some aspects, a polynucleic acid molecule comprises a sense strand and an antisense strand, wherein the antisense strand comprises a glyceryl modification at the 3′ end of the antisense strand.

In some aspects, a polynucleic acid molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19,20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′-and 5′-ends of the antisense strand. In other aspects, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate internucleotide linkages and/or a terminal cap molecule at the 3′-end, the 5′-end, or both of the 3′- and 5′-ends, being present in the same or different strand.

In some aspects, a polynucleic acid molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3′-end, the 5′-end, or both of the 3′- and 5′-ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′-and 5′-ends of the antisense strand. In other aspects, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages and/or a terminal cap molecule at the 3′-end, the 5′-end, or both of the 3′- and 5′-ends, being present in the same or different strand.

In some aspects, a polynucleic acid molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate internucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides at the 3′-end, the 5′-end, or both of the 3′- and 5′-ends of the sense strand and/or antisense strand, and optionally a terminal cap molecule at the 3′-end, the 5′-end, or both of the 3′- and 5′-ends of the sense strand. In some aspects, the antisense strand comprises about 1 to about 10 or more, specifically about 1, 2, 3, 4, 5, 6, 7, 8, 9, 10 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other aspects, one or more, for example about 1, 2, 3, 4, 5, 6, 7, 8, 9, 10, 11, 12, 13, 14, 15, 16, 17, 18, 19,20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate internucleotide linkages and/or a terminal cap molecule at the 3′-end, the 5′-end, or both of the 3′ and 5′-ends, being present in the same or different strand.

In some aspects, a polynucleic acid molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′-and 5′-ends of the antisense strand. In other aspects, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In some aspects, a polynucleic acid molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate internucleotide linkages in each strand of the polynucleic acid molecule. In some aspects, a polynucleic acid molecule comprises a sense strand and an antisense strand, and the antisense strand comprises a phosphate backbone modification at the 3′ end of the antisense strand. Alternatively and/or additionally, a polynucleic acid molecule comprises a sense strand and an antisense strand, and the sense strand comprises a phosphate backbone modification at the 5′ end of the antisense strand. In some instances, the phosphate backbone modification is a phosphorothioate. In some aspects, the sense or antisense strand has three consecutive nucleosides that are coupled via two phosphorothioate backbone.

In another embodiment, a polynucleic acid molecule described herein comprises 2′-5′ internucleotide linkages. In some instances, the 2′-5′ internucleotide linkage(s) is at the 3′-end, the 5′-end, or both of the 3′- and 5′-ends of one or both sequence strands. In addition instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the polynucleic acid molecule comprise a 2′-5′ internucleotide linkage, or about 1, 2, 3, 4, 5, 6, 7, 8, 9, 10, or more including every internucleotide linkage of a purine nucleotide in one or both strands of the polynucleic acid molecule comprise a 2′-5′ internucleotide linkage.

In some aspects, a polynucleic acid molecule is a single stranded polynucleic acid molecule that mediates RNAi activity in a cell or reconstituted in vitro system, wherein the polynucleic acid molecule comprises a single stranded polynucleotide having complementarity to a target nucleic acid sequence, and wherein one or more pyrimidine nucleotides present in the polynucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the polynucleic acid are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the polynucleic acid molecule optionally further comprising about 1 to about 4 (e.g., about 1, 2, 3, or 4) terminal 2′-deoxynucleotides at the 3′-end of the polynucleic acid molecule, wherein the terminal nucleotides further comprise one or more (e.g., 1, 2, 3, or 4) phosphorothioate internucleotide linkages, and wherein the polynucleic acid molecule optionally further comprises a terminal phosphate group, such as a 5′-terminal phosphate group.

In some cases, one or more of the artificial nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoroN3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified polynucleic acid molecule is nuclease resistance (e.g., RNase H, DNase, 5′-3′ exonuclease or 3′-5′ exonuclease resistance). In some instances, 2′-O-methoxyethyl (2′-O-MOE) modified polynucleic acid molecule is nuclease resistance (e.g., RNase H, DNase, 5′-3′ exonuclease or 3′-5′ exonuclease resistance). In some instances, 2′-O-aminopropyl modified polynucleic acid molecule is nuclease resistance (e.g., RNase H, DNase, 5′-3′ exonuclease or 3′-5′ exonuclease resistance). In some instances, 2′-deoxy modified polynucleic acid molecule is nuclease resistance (e.g., RNase H, DNase, 5′-3′ exonuclease or 3′-5′ exonuclease resistance). In some instances, 2′-deoxy-2′-fluoro modified polynucleic acid molecule is nuclease resistance (e.g., RNase H, DNase, 5′-3′ exonuclease or 3′-5′ exonuclease resistance). In some instances, 2′-O-aminopropyl (2′-O-AP) modified polynucleic acid molecule is nuclease resistance (e.g., RNase H, DNase, 5′-3′ exonuclease or 3′-5′ exonuclease resistance). In some instances, 2′-O-dimethylaminoethyl (2′-O-DMAOE) modified polynucleic acid molecule is nuclease resistance (e.g., RNase H, DNase, 5′-3′ exonuclease or 3′-5′ exonuclease resistance). In some instances, 2′-O-dimethylaminopropyl (2′-O-DMAP) modified polynucleic acid molecule is nuclease resistance (e.g., RNase H, DNase, 5′-3′ exonuclease or 3′-5′ exonuclease resistance). In some instances, 2′-O-dimethylaminoethyloxyethyl (2′-O-DMAEOE) modified polynucleic acid molecule is nuclease resistance (e.g., RNase H, DNase, 5′-3′ exonuclease or 3′-5′ exonuclease resistance). In some instances, 2′-O—N-methylacetamido (2′-O-NMA) modified polynucleic acid molecule is nuclease resistance (e.g., RNaseH, DNase, 5′-3′ exonuclease or 3′-5′ exonuclease resistance).

In some instances, LNA modified polynucleic acid molecule is nuclease resistance (e.g., RNase H, DNase, 5′-3′ exonuclease or 3′-5′ exonuclease resistance). In some instances, ENA modified polynucleic acid molecule is nuclease resistance (e.g., RNase H, DNase, 5′-3′ exonuclease or 3′-5′ exonuclease resistance). In some instances, HNA modified polynucleic acid molecule is nuclease resistance (e.g., RNaseH, DNase, 5′-3′ exonuclease or 3′-5′ exonuclease resistance).

In some instances, morpholinos is nuclease resistance (e.g., RNase H, DNase, 5′-3′ exonuclease or 3′-5′ exonuclease resistance). In some instances, PNA modified polynucleic acid molecule is resistant to nucleases (e.g., RNase H, DNase, 5′-3′ exonuclease or 3′-5′ exonuclease resistance).

In some instances, methylphosphonate nucleotides modified polynucleic acid molecule is nuclease resistance (e.g., RNaseH, DNase, 5′-3′ exonuclease or 3′-5′ exonuclease resistance).

In some instances, thiolphosphonate nucleotides modified polynucleic acid molecule is nuclease resistance (e.g., RNase H, DNase, 5′-3′ exonuclease or 3′-5′ exonuclease resistance). In some instances, polynucleic acid molecule comprising 2′-fluoroN3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In some aspects, one or more of the artificial nucleotide analogues described herein have increased binding affinity toward their mRNA target relative to an equivalent natural polynucleic acid molecule. The one or more of the artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified polynucleic acid molecule has increased binding affinity toward their mRNA target relative to an equivalent natural polynucleic acid molecule. In some instances, 2′-O-methoxyethyl (2′-O-MOE) modified polynucleic acid molecule has increased binding affinity toward their mRNA target relative to an equivalent natural polynucleic acid molecule. In some instances, 2′-O-aminopropyl modified polynucleic acid molecule has increased binding affinity toward their mRNA target relative to an equivalent natural polynucleic acid molecule. In some instances, 2′-deoxy modified polynucleic acid molecule has increased binding affinity toward their mRNA target relative to an equivalent natural polynucleic acid molecule. In some instances, 2′-deoxy-2′-fluoro modified polynucleic acid molecule has increased binding affinity toward their mRNA target relative to an equivalent natural polynucleic acid molecule. In some instances, 2′-O-aminopropyl (2′-O-AP) modified polynucleic acid molecule has increased binding affinity toward their mRNA target relative to an equivalent natural polynucleic acid molecule. In some instances, 2′-O-dimethylaminoethyl (2′-O-DMAOE) modified polynucleic acid molecule has increased binding affinity toward their mRNA target relative to an equivalent natural polynucleic acid molecule. In some instances, 2′-O-dimethylaminopropyl (2′-O-DMAP) modified polynucleic acid molecule has increased binding affinity toward their mRNA target relative to an equivalent natural polynucleic acid molecule. In some instances, 2′-O-dimethylaminoethyloxyethyl (2′-O-DMAEOE) modified polynucleic acid molecule has increased binding affinity toward their mRNA target relative to an equivalent natural polynucleic acid molecule. In some instances, 2′-O—N-methylacetamido (2′-O-NMA) modified polynucleic acid molecule has increased binding affinity toward their mRNA target relative to an equivalent natural polynucleic acid molecule. In some instances, LNA modified polynucleic acid molecule has increased binding affinity toward their mRNA target relative to an equivalent natural polynucleic acid molecule. In some instances, ENA modified polynucleic acid molecule has increased binding affinity toward their mRNA target relative to an equivalent natural polynucleic acid molecule. In some instances, PNA modified polynucleic acid molecule has increased binding affinity toward their mRNA target relative to an equivalent natural polynucleic acid molecule. In some instances, HNA modified polynucleic acid molecule has increased binding affinity toward their mRNA target relative to an equivalent natural polynucleic acid molecule. In some instances, morpholino modified polynucleic acid molecule has increased binding affinity toward their mRNA target relative to an equivalent natural polynucleic acid molecule. In some instances, methylphosphonate nucleotides modified polynucleic acid molecule has increased binding affinity toward their mRNA target relative to an equivalent natural polynucleic acid molecule. In some instances, thiolphosphonate nucleotides modified polynucleic acid molecule has increased binding affinity toward their mRNA target relative to an equivalent natural polynucleic acid molecule. In some instances, polynucleic acid molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some aspects, a polynucleic acid molecule described herein is a chirally pure (or stereo pure) polynucleic acid molecule, or a polynucleic acid molecule comprising a single enantiomer. In some instances, the polynucleic acid molecule comprises L-nucleotide. In some instances, the polynucleic acid molecule comprises D-nucleotides. In some instance, a polynucleic acid molecule composition comprises less than 30%, 25%, 20%, 15%, 10%, 5%, 4%, 3%, 2%, 1%, or less of its mirror enantiomer. In some cases, a polynucleic acid molecule composition comprises less than 30%, 25%, 20%, 15%, 10%, 5%, 4%, 3%, 2%, 1%, or less of a racemic mixture. In some instances, the polynucleic acid molecule is a polynucleic acid molecule described in: U.S. Patent Publication Nos: 2014/194610 and 2015/211006; and PCT Publication No: WO2015107425.

In some aspects, a polynucleic acid molecule described herein is further modified to include an aptamer conjugating moiety. In some instances, the aptamer conjugating moiety is a DNA aptamer conjugating moiety. In some instances, the aptamer conjugating moiety is Alphamer (Centauri Therapeutics), which comprises an aptamer portion that recognizes a specific cell-surface target and a portion that presents a specific epitopes for attaching to circulating antibodies. In some instance, a polynucleic acid molecule described herein is further modified to include an aptamer conjugating moiety as described in: U.S. Pat. Nos: 8,604,184, 8,591,910, and 7,850,975.

In additional aspects, a polynucleic acid molecule described herein is modified to increase its stability. In some embodiment, the polynucleic acid molecule is RNA (e.g., siRNA).

In some instances, the polynucleic acid molecule is modified by one or more of the modifications described above to increase its stability. In some cases, the polynucleic acid molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′- O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the polynucleic acid molecule is modified by 2′-O-methyl and/or 2′-O-methoxyethyl ribose. In some cases, the polynucleic acid molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

In some instances, the polynucleic acid molecule is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. In some instances, the polynucleic acid molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the polynucleic acid molecule is assembled from a single oligonucleotide, where the self-complementary sense and antisense regions of the polynucleic acid molecule are linked by means of a nucleic acid based or non-nucleic acid-based linker(s).

In some cases, the polynucleic acid molecule is a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. In other cases, the polynucleic acid molecule is a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide is processed either in vivo or in vitro to generate an active polynucleic acid molecule capable of mediating RNAi. In additional cases, the polynucleic acid molecule also comprises a single-stranded polynucleotide having nucleotide sequence complementary to nucleotide sequence in a target nucleic acid molecule or a portion thereof (for example, where such polynucleic acid molecule does not require the presence within the polynucleic acid molecule of nucleotide sequence corresponding to the target nucleic acid sequence or a portion thereof), wherein the single stranded polynucleotide further comprises a terminal phosphate group, such as a 5′-phosphate, or 5′, 3′-diphosphate.

In some instances, an asymmetric hairpin is a linear polynucleic acid molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin polynucleic acid molecule comprises an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. In some cases, the asymmetric hairpin polynucleic acid molecule also comprises a 5′-terminal phosphate group that is chemically modified. In additional cases, the loop portion of the asymmetric hairpin polynucleic acid molecule comprises nucleotides, non-nucleotides, linker molecules, or conjugate molecules.

In some aspects, an asymmetric duplex is a polynucleic acid molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex polynucleic acid molecule comprises an antisense region having length sufficient to mediate RNAi in a cell or in vitro system (e.g., about 19 to about 22 nucleotides) and a sense region having about 3 to about 18 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art.

### Polynucleic Acid Molecule Synthesis

In some aspects, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication NO. 1579015. Additional exemplary methods include those described in: Griffey et al., “2′-O-aminopropyl ribonucleotides: a zwitterionic modification that enhances the exonuclease resistance and biological activity of antisense oligonucleotides,” J. Med Chem. 39(26):5100-5109 (1997)); Obika, et al. “Synthesis of 2′-0,4′-C-methyleneuridine and -cytidine. Novel bicyclic nucleosides having a fixed C3, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B:1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some aspects, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, a polynucleic acid molecule is also assembled from two distinct nucleic acid strands or fragments wherein one fragment includes the sense region and the second fragment includes the antisense region of the molecule.

Additional modification methods for incorporating, for example, sugar, base and phosphate modifications include: Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010.

Such publications describe general methods and strategies to determine the location of incorporation of sugar, base and/or phosphate modifications and the like into nucleic acid molecules without modulating catalysis.

In some instances, while chemical modification of the polynucleic acid molecule internucleotide linkages with phosphorothioate, phosphorodithioate, and/or 5′-methylphosphonate linkages improves stability, excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

### Polynucleic Acid Molecule Conjugates

In some aspects, a polynucleic acid molecule (B) is further conjugated to a polypeptide (A) for delivery to a site of interest. In some instances, at least one polypeptide A is conjugated to at least one B. In some instances, the at least one polypeptide A is conjugated to the at least one B to form an A-B conjugate. In some aspects, at least one A is conjugated to the 5′ terminus of B, the 3′ terminus of B, an internal site on B, or in any combinations thereof. In some instances, the at least one polypeptide A is conjugated to at least two B. In some instances, the at least one polypeptide A is conjugated to at least 2, 3, 4, 5, 6, 7, 8, or more B.

In some cases, a polynucleic acid molecule is conjugated to a polypeptide (A) and optionally a polymeric moiety (C). In some aspects, at least one polypeptide A is conjugated at one terminus of at least one B while at least one C is conjugated at the opposite terminus of the at least one B to form an A-B-C conjugate. In some instances, at least one polypeptide A is conjugated at one terminus of the at least one B while at least one of C is conjugated at an internal site on the at least one B. In some instances, at least one polypeptide A is conjugated directly to the at least one C. In some instances, the at least one B is conjugated indirectly to the at least one polypeptide A via the at least one C to form an A-C-B conjugate.

In some instances, at least one B and/or at least one C, and optionally at least one D are conjugated to at least one polypeptide A. In some instances, the at least one B is conjugated at a terminus (e.g., a 5′ terminus or a 3′ terminus) to the at least one polypeptide A or are conjugated via an internal site to the at least one polypeptide A. In some cases, the at least one C is conjugated either directly to the at least one polypeptide A or indirectly via the at least one B. If indirectly via the at least one B, the at least one C is conjugated either at the same terminus as the at least one polypeptide A on B, at opposing terminus from the at least one polypeptide A, or independently at an internal site. In some instances, at least one additional polypeptide A is further conjugated to the at least one polypeptide A, to B, or to C. In additional instances, the at least one D is optionally conjugated either directly or indirectly to the at least one polypeptide A, to the at least one B, or to the at least one C. If directly to the at least one polypeptide A, the at least one D is also optionally conjugated to the at least one B to form an A-D-B conjugate or is optionally conjugated to the at least one B and the at least one C to form an A-D-B-C conjugate. In some instances, the at least one D is directly conjugated to the at least one polypeptide A and indirectly to the at least one B and the at least one C to form a D-A-B-C conjugate. If indirectly to the at least one polypeptide A, the at least one D is also optionally conjugated to the at least one B to form an A-B-D conjugate or is optionally conjugated to the at least one B and the at least one C to form an A-B-D-C conjugate. In some instances, at least one additional D is further conjugated to the at least one polypeptide A, to B, or to C.

### Binding Moiety

In some aspects, the binding moiety A is a polypeptide. In some instances, the polypeptide is an antibody or its fragment thereof. In some cases, the fragment is an antigen binding fragment. In some instances, the antibody or antigen binding fragment thereof comprises a humanized antibody or antigen binding fragment thereof, murine antibody or antigen binding fragment thereof, chimeric antibody or antigen binding fragment thereof, monoclonal antibody or antigen binding fragment thereof, a binding fragment having a light chain domain and a heavy chain domain, a binding fragment having two light chain domains and two heavy chain domains, a binding fragment having two or more light chain domains and heavy chain domains, monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, or a chemically modified derivative thereof.

In some aspects, the binding moiety A is a bispecific antibody or antigen binding fragment thereof. In some instances, the bispecific antibody is a trifunctional antibody or a bispecific mini-antibody. In some cases, the bispecific antibody is a trifunctional antibody. In some instances, the trifunctional antibody is a full length monoclonal antibody comprising binding sites for two different antigens.

In some cases, the bispecific antibody is a bispecific mini-antibody. In some instances, the bispecific mini-antibody comprises divalent Fab₂, F(ab)′₃ fragments, bis-scFv, (scFv)₂, diabody, minibody, triabody, tetrabody or a bi-specific T-cell engager (BiTE). In some aspects, the bi-specific T-cell engager is a fusion protein that contains two single-chain variable fragments (scFvs) in which the two scFvs target epitopes of two different antigens.

In some aspects, the binding moiety A is a bispecific mini-antibody. In some instances, A is a bispecific Fab₂. In some instances, A is a bispecific F(ab)′₃ fragment. In some cases, A is a bispecific bis-scFv. In some cases, A is a bispecific (scFv)₂. In some aspects, A is a bispecific diabody. In some aspects, A is a bispecific minibody. In some aspects, A is a bispecific triabody.

In other aspects, A is a bispecific tetrabody. In other aspects, A is a bi-specific T-cell engager (BiTE).

In some aspects, the binding moiety A is a trispecific antibody. In some instances, the trispecific antibody comprises F(ab)′₃ fragments or a triabody. In some instances, A is a trispecific F(ab)′₃ fragment. In some cases, A is a triabody. In some aspects, A is a trispecific antibody as described in Dimas, et al., “Development of a trispecific antibody designed to simultaneously and efficiently target three different antigens on tumor cells,” Mol.

### Pharmaceutics, 12(9): 3490-3501 (2015).

In some aspects, the binding moiety A is an antibody or antigen binding fragment thereof that recognizes a cell surface protein. In some instances, the binding moiety A is an antibody or antigen binding fragment thereof that recognizes a cell surface protein on a muscle cell. In some cases, the binding moiety A is an antibody or antigen binding fragment thereof that recognizes a cell surface protein on a skeletal muscle cell.

In some aspects, exemplary antibodies include, but are not limited to, an anti-myosin antibody, an anti-transferrin receptor antibody, and an antibody that recognizes Muscle-Specific kinase (MuSK). In some instances, the antibody is an anti-transferrin receptor (anti-CD71) antibody.

In some aspects, where the antibody is an anti-transferrin receptor (anti-CD71) antibody, the anti-transferrin antibody specifically binds to a transferrin receptor (TfR), preferably, specifically binds to transferrin receptor 1 (TfR1), or more preferably, specifically binds to human transferrin receptor 1 (TfR1) (or human CD71).

In some instances, the anti-transferrin receptor antibody comprises a variable heavy chain (VH) region and a variable light chain (VL) region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283.

In some aspects, the VH region of the anti-transferrin receptor antibody comprises HCDR1, HCDR2, and HCDR3 sequences selected from Table 1.

TABLE 1

| | | SEQ | | SEQ | | SEQ |
|---|---|---|---|---|---|---|
| Name | HCDR1 | ID NO: | HCDR2 | ID NO: | HCDR3 | ID NO: |
| 13E4_VH1 | YTFTNYWM | 281 | EINPINGRSNYAQKFQ | 282 | GTRAMH | 283 |
| | H | | G | | Y | |
| 13E4_VH2* | YTFTNYWM | 281 | EINPINGRSNYAEKFQ | 284 | GTRAMH | 283 |
| | H | | G | | Y | |
| 13E4_VH3 | YTFTNYWM | 281 | EINPIQGRSNYAEKFQ | 285 | GTRAMH | 283 |
| | H | | G | | Y | |

*13E4_VH2 shares the ame HCR1, HCDR2, and HCDR3 sequences with anti-transferrin receptor antibody 13E4_VH4

In some aspects, the VH region comprises HCDR1 sequence comprising SEQ ID NO: 281; HCDR2 sequence comprising SEQ ID NO: 282, 284, or 285; and HCDR3 sequence comprising SEQ ID NO: 283. In some instances, the VH region comprises HCDR1 sequence comprising SEQ ID NO: 281, HCDR2 sequence comprising SEQ ID NO: 282, and HCDR3 sequence comprising SEQ ID NO: 283. In some instances, the VH region comprises HCDR1 sequence comprising SEQ ID NO: 281, HCDR2 sequence comprising SEQ ID NO: 284, and HCDR3 sequence comprising SEQ ID NO: 283. In some instances, the VH region comprises HCDR1 sequence comprising SEQ ID NO: 281, HCDR2 sequence comprising SEQ ID NO: 285, and HCDR3 sequence comprising SEQ ID NO: 283.

In some aspects, the VL region of the anti-transferrin receptor antibody comprises LCDR1 sequence RTSENIYX₃NLA (SEQ ID NO: 407), LCDR2 sequence AX₄TNLAX₅(SEQ ID NO: 408), and LCDR3 sequence QHFWGTPLTX₆ (SEQ ID NO: 409), wherein X₃ is selected from N or S, X₄ is selected from A or G, X₅ is selected from D or E, and X₆ is present or absence, and if present, is F.

In some aspects, the VL region of the anti-transferrin receptor antibody comprises LCDR1, LCDR2, and LCDR3 sequences selected from Table 2.

TABLE 2

| | | SEQ | | SEQ | | SEQ |
|---|---|---|---|---|---|---|
| Name | LCDR1 | ID NO: | LCDR2 | ID NO: | LCDR3 | ID NO: |
| 13E4_VL1* | RTSENIYNNLA | 286 | AATNLAD | 287 | QHFWGTPLT | 288 |
| 13E4_VL3 | RTSENIYNNLA | 286 | AATNLAE | 289 | QHFWGTPLTF | 290 |
| 13E4_VL4 | RTSENIYSNLA | 291 | AGTNLAD | 292 | QHFWGTPLTF | 290 |

*13E4_VL1 shares the same LCDR1, LCDR2, and LCDR3 sequences with anti-transferrin receptor antibody 13E4_VL2

In some instances, the VL region comprises LCDR1 sequence RTSENIYX₃NLA (SEQ ID NO: 407), LCDR2 sequence comprising SEQ ID NO: 287, 289, or 292, and LCDR3 sequence comprising SEQ ID NO: 288 or 290, wherein X₃ is selected from N or S.

In some instances, the VL region comprises LCDR1 sequence comprising SEQ ID NO: 286 or 291, LCDR2 sequence AX₄TNLAX₅(SEQ ID NO: 408), and LCDR3 sequence comprising SEQ ID NO: 288 or 290, wherein X₄ is selected from A or G, and X₅ is selected from D or E.

In some instances, the VL region comprises LCDR1 sequence comprising SEQ ID NO: 286 or 291, LCDR2 sequence SEQ ID NO: 287, 289, or 292, and LCDR3 sequence QHFWGTPLTX₆ (SEQ ID NO: 409), wherein X₆ is present or absence, and if present, is F.

In some instances, the VL region comprises LCDR1 sequence comprising SEQ ID NO: 286, LCDR2 sequence AATNLAX5 (SEQ ID NO: 410), and LCDR3 sequence QHFWGTPLTX₆ (SEQ ID NO: 409), wherein X₅ is selected from D or E and X₆ is present or absence, and if present, is F.

In some instances, the VL region comprises LCDR1 sequence comprising SEQ ID NO: 286, LCDR2 sequence comprising SEQ ID NO: 287, and LCDR3 sequence comprising SEQ ID NO: 288.

In some instances, the VL region comprises LCDR1 sequence comprising SEQ ID NO: 286, LCDR2 sequence comprising SEQ ID NO: 289, and LCDR3 sequence comprising SEQ ID NO: 290.

In some instances, the VL region comprises LCDR1 sequence comprising SEQ ID NO: 291, LCDR2 sequence comprising SEQ ID NO: 292, and LCDR3 sequence comprising SEQ ID NO: 290.

In some aspects, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence RTSENIYX₃NLA (SEQ ID NO: 407), LCDR2 sequence AX₄TNLAX₅(SEQ ID NO: 408), and LCDR3 sequence QHFWGTPLTX₆ (SEQ ID NO: 409), wherein X₃ is selected from N or S, X₄ is selected from A or G, X₅ is selected from D or E, and X₆ is present or absence, and if present, is F.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence RTSENIYX₃NLA (SEQ ID NO: 407), LCDR2 sequence comprising SEQ ID NO: 287, 289, or 292, and LCDR3 sequence comprising SEQ ID NO: 288 or 290, wherein X₃ is selected from N or S.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence comprising SEQ ID NO: 286 or 291, LCDR2 sequence AX₄TNLAX₅(SEQ ID NO: 408), and LCDR3 sequence comprising SEQ ID NO: 288 or 290, wherein X₄ is selected from A or G, and X₅ is selected from D or E.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX₆ (SEQ ID NO: 409), wherein X₆ is present or absence, and if present, is F.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence comprising SEQ ID NO: 286, LCDR2 sequence AATNLAX5 (SEQ ID NO: 410), and LCDR3 sequence QHFWGTPLTX₆ (SEQ ID NO: 409), wherein X₅ is selected from D or E and X₆ is present or absence, and if present, is F.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO: 290.

In some instances, the anti-transferrin receptor antibody comprises a VH region and a VL region, wherein the VH region comprises HCDR1 sequence comprising SEQ ID NO: 281; HCDR2 sequence EINPIX₁GRSNYAX₂KFQG (SEQ ID NO: 406), wherein X₁ is selected from N or Q and X₂ is selected from Q or E; and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence RTSENIYX₃NLA (SEQ ID NO: 407), LCDR2 sequence comprising SEQ ID NO: 287, 289, or 292, and LCDR3 sequence comprising SEQ ID NO: 288 or 290, wherein X₃ is selected from N or S.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286 or 291, LCDR2 sequence AX₄TNLAX₅(SEQ ID NO: 408), and LCDR3 sequence comprising SEQ ID NO: 288 or 290, wherein X₄ is selected from A or G, and X₅ is selected from D or E.

In some instances, the anti-transferrin receptor antibody comprises a VH region and a VL region, in which the VH region comprises HCDR1 sequence comprising SEQ ID NO: 281, HCDR2 sequence comprising SEQ ID NO: 2, and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX₆ (SEQ ID NO: 409), wherein X₆ is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286, LCDR2 sequence AATNLAX5 (SEQ ID NO: 410), and LCDR3 sequence QHFWGTPLTX₆ (SEQ ID NO: 409), wherein X₅ is selected from D or E and X₆ is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 282, and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence RTSENIYX₃NLA (SEQ ID NO: 407), LCDR2 sequence comprising SEQ ID NO: 287, 289, or 292, and LCDR3 sequence comprising SEQ ID NO: 288 or 290, wherein X₃ is selected from N or S.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286 or 291, LCDR2 sequence AX₄TNLAX₅(SEQ ID NO: 408), and LCDR3 sequence comprising SEQ ID NO: 288 or 290, wherein X₄ is selected from A or G, and X₅ is selected from D or E.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX₆ (SEQ ID NO: 409), wherein X₆ is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286, LCDR2 sequence AATNLAX5 (SEQ ID NO: 410), and LCDR3 sequence QHFWGTPLTX₆ (SEQ ID NO: 409), wherein X₅ is selected from D or E and X₆ is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO:290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 284, and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence RTSENIYX₃NLA (SEQ ID NO: 407), LCDR2 sequence comprising SEQ ID NO: 287, 289, or 292, and LCDR3 sequence comprising SEQ ID NO: 288 or 290, wherein X₃ is selected from N or S.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286 or 291, LCDR2 sequence AX₄TNLAX₅(SEQ ID NO: 408), and LCDR3 sequence comprising SEQ ID NO: 288 or 290, wherein X₄ is selected from A or G, and X₅ is selected from D or E.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286 or 291, LCDR2 sequence SEQ ID NO: 287, 289, or 292, and LCDR3 sequence QHFWGTPLTX₆ (SEQ ID NO: 409), wherein X₆ is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283 and the VL region comprises LCDR1 sequence comprising SEQ ID NO: 286, LCDR2 sequence AATNLAX5 (SEQ ID NO: 410), and LCDR3 sequence QHFWGTPLTX₆ (SEQ ID NO: 409), wherein X₅ is selected from D or E and X₆ is present or absence, and if present, is F.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286, LCDR2 sequence comprising SEQ ID NO: 287, and LCDR3 sequence comprising SEQ ID NO: 288.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 286, LCDR2 sequence comprising SEQ ID NO: 289, and LCDR3 sequence comprising SEQ ID NO: 290.

In some instances, the anti-transferrin receptor antibody comprises a VH region and a VL region, in which the VH region comprises HCDR1 sequence comprising SEQ ID NO: 281, HCDR2 sequence comprising SEQ ID NO: 285, and HCDR3 sequence comprising SEQ ID NO: 283; and the VL region comprises LCDR1 sequence comprising SEQ ID NO: 291, LCDR2 sequence comprising SEQ ID NO: 292, and LCDR3 sequence comprising SEQ ID NO: 290.

In some aspects, the anti-transferring receptor comprises a VH region and a VL region in which the sequence of the VH region comprises about 80%, 85%, 90%, 95%, 96%, 97%, 98%, 99%, or 100% sequence identity to SEQ ID NOs: 293-296 and the sequence of the VL region comprises about 80%, 85%, 90%, 95%, 96%, 97%, 98%, 99%, 100% sequence identity to SEQ ID NOs: 298-301

TABLE 3

| NAME | VH SEQUENCE | SEQ ID NO: |
|---|---|---|
| 13E4_VH1 | QVQLVQSGAEVKKPGASVKVSCKASGYTFTNYWMHWVRQAPGQ | 293 |
| | GLEWMGEINPINGRSNYAQKFQGRVTLTVDTSISTAYMELSRLRSD | |
| | DTAVYYCARGTRAMHYWGQGTLVTVSS | |
| 13E4_VH2 | QVQLVQSGAEVKKPGASVKVSCKASGYTFTNYWMHWVRQAPGQ | 294 |
| | GLEWIGEINPINGRSNYAEKFQGRVTLTVDTSSSTAYMELSRLRSD | |
| | DTAVYYCARGTRAMHYWGQGTLVTVSS | |
| 13E4_VH3 | QVQLVQSGAEVKKPGASVKVSCKASGYTFTNYWMHWVRQAPGQ | 295 |
| | GLEWMGEINPIQGRSNYAEKFQGRVTLTVDTSSSTAYMELSSLRSE | |
| | DTATYYCARGTRAMHYWGQGTLVTVSS | |
| 13E4_VH4 | QVQLVQSGAEVKKPGASVKVSCKASGYTFTNYWMHWVRQAPGQ | 296 |
| | GLEWMGEINPINGRSNYAEKFQGRVTLTVDTSSSTAYMELSSLRSE | |
| | DTATYYCARGTRAMHYWGQGTLVTVSS | |
| 13E4_VH | QVQLQQPGAELVKPGASVKLSCKASGYTFTNYWMHWVKQRPGQ | 297 |
| | GLEWIGEINPINGRSNYGERFKTKATLTVDKSSSTAYMQLSSLTSED | |
| | SAVYYCARGTRAMHYWGQGTSVTVSS | |

TABLE 4

| | | SEQ ID |
|---|---|---|
| NAME | VL SEQUENCE | NO: |
| 13E4_VL1 | DIQMTQSPSSLSASVGDRVTITCRTSENIYNNLAWYQQKPGKSPKL | 298 |
| | LIYAATNLADGVPSRFSGSGSGTDYTLTISSLQPEDFATYYCQHFW | |
| | GTPLTFGGGTKVEIK | |
| 13E4_VL2 | DIQMTQSPSSLSASVGDRVTITCRTSENIYNNLAWYQQKPGKAPKL | 299 |
| | LIYAATNLADGVPSRFSGSGSGTDYTLTISSLQPEDFATYYCQHFW | |
| | GTPLTFGGGTKVEIK | |
| 13E4_VL3 | DIQMTQSPSSLSASVGDRVTITCRTSENIYNNLAWYQQKPGKAPKL | 300 |
| | LIYAATNLAEGVPSRFSGSGSGTDYTLTISSLQPEDFATYYCQHFW | |
| | GTPLTFGGGTKVEIK | |
| 13E4_VL4 | DIQMTQSPSSLSASVGDRVTITCRTSENIYSNLAWYQQKPGKAPKL | 301 |
| | LIYAGTNLADGVPSRFSGSGSGTDYTLTISSLQPEDFANYYCQHFW | |
| | GTPLTFGGGTKVEIK | |
| 13E4_VL | DIQMTQSPASLSVSVGETVTITCRTSENIYNNLAWYQQKQGKSPQL | 302 |
| | LVYAATNLADGVPSRFSGSGSGTQYSLKINSLQSEDFGNYYCQHF | |
| | WGTPLTFGAGTKLELK | |

In some aspects, the anti-transferrin receptor antibody comprises a VH region and a VL region as illustrated in Table 5.

TABLE 5

| | 13E4_VH1 | 13E4_VH2 | 13E4_VH3 | 13E4_VH4 |
|---|---|---|---|---|
| | (SEQ ID | (SEQ ID | (SEQ ID | (SEQ ID |
| | NO: 293) | NO: 294) | NO: 295) | NO: 296) |
| 13E4_VL1 | SEQ ID NO: | SEQ ID NO: | SEQ ID NO: | SEQ ID NO: |
| (SEQ ID | 293 + | 294 + | 295 + | 296 + |
| NO: 298) | SEQ ID NO: | SEQ ID NO: | SEQ ID NO: | SEQ ID NO: |
| | 298 | 298 | 298 | 298 |
| 13E4_VL2 | SEQ ID NO: | SEQ ID NO: | SEQ ID NO: | SEQ ID NO: |
| (SEQ ID | 293 + | 294 + | 295 + | 296 + |
| NO: 299) | SEQ ID NO: | SEQ ID NO: | SEQ ID NO: | SEQ ID NO: |
| | 299 | 299 | 299 | 299 |
| 13E4_VL3 | SEQ ID NO: | SEQ ID NO: | SEQ ID NO: | SEQ ID NO: |
| (SEQ ID | 293 + | 294 + | 295 + | 296 + |
| NO: 300) | SEQ ID NO: | SEQ ID NO: | SEQ ID NO: | SEQ ID NO: |
| | 300 | 300 | 300 | 300 |
| 13E4_VL4 | SEQ ID NO: | SEQ ID NO: | SEQ ID NO: | SEQ ID NO: |
| (SEQ ID | 293 + | 294 + | 295 + | 296 + |
| NO: 301) | SEQ ID NO: | SEQ ID NO: | SEQ ID NO: | SEQ ID NO: |
| | 301 | 301 | 301 | 301 |

In some aspects, an anti-transferrin receptor antibody described herein comprises an IgG framework, an IgA framework, an IgE framework, or an IgM framework. In some instances, the anti-transferrin receptor antibody comprises an IgG framework (e.g., IgG1, IgG2, IgG3, or IgG4). In some cases, the anti-transferrin receptor antibody comprises an IgG1 framework. In some cases, the anti-transferrin receptor antibody comprises an IgG2 (e.g., an IgG2a or IgG2b) framework. In some cases, the anti-transferrin receptor antibody comprises an IgG2a framework. In some cases, the anti-transferrin receptor antibody comprises an IgG2b framework. In some cases, the anti-transferrin receptor antibody comprises an IgG3 framework.

In some cases, the anti-transferrin receptor antibody comprises an IgG4 framework.

In some cases, an anti-transferrin receptor antibody comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC).

In additional instances, the one or more mutations are to modulate glycosylation. In some aspects, the one or more mutations are located in the Fc region. In some instances, the Fc region comprises a mutation at residue position L234, L235, or a combination thereof. In some instances, the mutations comprise L234 and L235. In some instances, the mutations comprise L234A and L235A. In some cases, the residue positions are in reference to IgG1.

In some instances, the Fc region comprises a mutation at residue position L234, L235, D265, N297, K322, L328, or P329, or a combination thereof. In some instances, the mutations comprise L234 and L235 in combination with a mutation at residue position K322, L328, or P329. In some cases, the Fc region comprises mutations at L234, L235, and K322. In some cases, the Fc region comprises mutations atL234, L235, andL328. In some cases, the Fc region comprises mutations atL234, L235, andP329. In some cases, the Fc region comprises mutations at D265 and N297. In some cases, the residue position is in reference to IgG1.

In some instances, the Fc region comprises L234A, L235A, D265A, N297G, K322G, L328R, or P329G, or a combination thereof. In some instances, the Fc region comprises L234A and L235A in combination with K322G, L328R, or P329G. In some cases, the Fc region comprises L234A, L235A, andK322G. In some cases, the Fc region comprises L234A, L235A, and L328R. In some cases, the Fc region comprises L234A, L235A, andP329G. In some cases, the Fc region comprises D265A andN297G. In some cases, the residue position is in reference to IgG1.

In some instances, the Fc region comprises a mutation at residue position L235, L236, D265, N297, K322, L328, or P329, or a combination of the mutations. In some instances, the Fc region comprises mutations at L235 and L236. In some instances, the Fc region comprises mutations atL235 and L236 in combinationwith a mutation atresidue position K322, L328, or P329. In some cases, the Fc region comprises mutations atL235, L236, and K322. In some cases, the Fc region comprises mutations atL235, L236, andL328. In some cases, the Fc region comprises mutations atL235, L236, and P329. In some cases, the Fc region comprises mutations at D265 and N297. In some cases, the residue position is in reference to IgG2b.

In some aspects, the Fc region comprises L235A, L236A, D265A, N297G, K322G, L328R, or P329G, or a combination thereof. In some instances, the Fc region comprises L235A and L236A. In some instances, the Fc region comprises L235A and L236A in combination with K322G, L328R, or P329G. In some cases, the Fc region comprises L235A, L236A, and K322G.

In some cases, the Fc region comprises L235A, L236A, and L328R. In some cases, the Fc region comprises L235A, L236A, and P329G. In some cases, the Fc region comprises D265A and N297G. In some cases, the residue position is in reference to IgG2b.

In some aspects, the Fc region comprises a mutation at residue position L233, L234, D264, N296, K321, L327, or P328, wherein the residues correspond to positions 233, 234, 264, 296, 321, 327, and 328 of SEQ ID NO: 303. In some instances, the Fc region comprises mutations at L233 and L234. In some instances, the Fc region comprises mutations at L233 and L234 in combination with a mutation at residue position K321, L327, or P328. In some cases, the Fc region comprises mutations at L233, L234, and K321. In some cases, the Fc region comprises mutations at L233, L234, and L327. In some cases, the Fc region comprises mutations atL233, L234, and K321. In some cases, the Fc region comprises mutations at L233, L234, and P328. In some instances, the Fc region comprises mutations at D264 and N296. In some cases, equivalent positions to residueL233, L234, D264, N296, K321, L327, orP328 in an IgG1, IgG2, IgG3, or IgG4 framework are contemplated. In some cases, mutations to a residue that corresponds to residue L233, L234, D264, N296, K321, L327, or P328 of SEQ ID NO: 23 in an IgG1, IgG2, or IgG4 framework are also contemplated.

In some aspects, the Fc region comprises L233A, L234A, D264A, N296G, K321G, L327R, or P328G, wherein the residues correspond to positions 233, 234, 264, 296, 321, 327, and 328 of SEQ ID NO: 303. In some instances, the Fc region comprises L233A and L234A. In some instances, the Fc region comprises L233A and L234A in combination with K321G, L327R, or P328G. In some cases, the Fc region comprises L233A, L234A, and K321G. In some cases, the Fc region comprises L233A, L234A, and L327R. In some cases, the Fc region comprises L233A, L234A, andK321G. In some cases, the Fc region comprises L233A, L234A, and P328G. In some instances, the Fc region comprises D264A and N296G.

In some aspects, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some aspects, an anti-transferrin receptor antibody described herein is a full-length antibody, comprising a heavy chain (HC) and a light chain (LC). In some cases, the heavy chain (HC) comprises a sequence selected from Table 6. In some cases, the light chain (LC) comprises a sequence selected from Table 7. The underlined region denotes the respective CDRs.

TABLE 6

| | | SEQ ID |
|---|---|---|
| NAME | HC SEQUENCE | NO: |
| 13E4_VH1 | QVQLVQSGAEVKKPGASVKVSCKASGYTFINYWMHWVRQAPGQ | 303 |
| | GLEWMGEINPINGRSNYAQKFQGRVTLTVDTSISTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH1_a | QVQLVQSGAEVKKPGASVKVSCKASGYTFTNYWMHWVRQAPGQ | 304 |
| | GLEWMGEINPINGRSNYAQKFQGRVTLTVDTSISTAYMELSRLRSD | |
| | DTAVYYCARGTRAMHYWGQGTLVTVSSASTKQPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH1_b | QVQLVQSGAEVKKPGASVKVSCKASGYTFINYWMHWVRQAPGQ | 305 |
| | GLEWMGEINPINGRSNYAQKFQGRVTLTVDTSISTAYMELSRLRSD | |
| | DTAVYYCARGTRAMHYWGQGTLVTVSSASTKGPSVFPLAPSSKST | |
| | SGGTAALQCLVKDYFPEPVTVSWNSGALTSQVHTFPAVIQSSQLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KENWYVDGVEVHNAKTKPREEQYNSTYRVVSVLTVLHQDWLNG | |
| | KEYKCGVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH1_c | QVQLVQSGAEVKKPGASVKVSCKASGYTFTNYWMHWVRQAPGQ | 306 |
| | GLEWMGEINPINGRSNYAQKFQGRVTLTVDTSTSTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKARPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH1_d | QVQLVQSGAEVKKPGASVKVSCKASGYTFINYWMHWVRQAPGQ | 307 |
| | GLEWMGEINPINGRSNYAQKFQGRVTLTVDTSISTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALGAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH1_e | QVQLVQSGAEVKKPGASVKVSCKASGYTFTNYWMHWVRQAPGQ | 308 |
| | GLEWMGEINPINGRSNYAQKFQGRVTLTVDTSISTAYMELSRLRSD | |
| | DTAVYYCARGTRAMHYWGQGTLVTVSSASTKGPSVFPLAPSSKST | |
| | SGGTAALQCLVKDYFPEPVTVSWNSGALTSQVHTFPAVIQSSQLY | |
| | SLSSVVTVPSSSLGTQTYICNVNHKPSNTKVDKRVEPKSCDKTHTC | |
| | PPCPAPELLGGPSVFLFPPKPKDTLMISRTPEVTCVVVAVSHEDPEV | |
| | KFNWYVDGVEVHNAKTKPREEQYGSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDQSFFLYS | |
| | KLTVDKSRWQQGNVFSCSVMHEALHNHYTQKSLSLSPG | |
| 13E4_VH2 | QVQLVQSGAEVKKPGASVKVSCKASGYTFTNYWMHWVRQAPGQ | 309 |
| | GLEWIGEINPINGRSNYAEKFQGRVTLTVDTSSSTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYTCNVNHKPSNTKVDKRVEPKSCDKTHTC | |
| | PPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH2_a | QVQLVQSGAEVKKPGASVKVSCKASGYTFINYWMHWVRQAPGQ | 310 |
| | GLEWIGEINPINGRSNYAEKFQGRVTLTVDTSSSTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVIINAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH2_b | QVQLVQSGAEVKKPGASVKVSCKASGYTFTNYWMHWVRQAPGQ | 311 |
| | GLEWIGEINPINGRSNYAEKFQGRVTLTVDTSSSTAYMELSRLRSD | |
| | DTAVYYCARGTRAMHYWGQGTLVTVSSASTKQPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCGVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH2_c | QVQLVQSGAEVKKPGASVKVSCKASGYTFTNYWMHWVRQAPGQ | 312 |
| | GLEWIGEINPINGRSNYAEKFQGRVTLTVDTSSSTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKARPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH2_d | QVQLVQSGAEVKKPGASVKVSCKASGYTFTNYWMHWVRQAPGQ | 313 |
| | GLEWIGEINPINGRSNYAEKFQGRVTLTVDTSSSTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALGAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH2_e | QVQLVQSGAEVKKPGASVKVSCKASGYTFTNYWMHWVRQAPGQ | 314 |
| | GLEWIGEINPINGRSNYAEKFQGRVTLTVDTSSSTAYMELSRLRSD | |
| | DTAVYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPELLGGPSVFLFPPKPKDTLMISRTPEVTCVVVAVSHEDPEV | |
| | KFNWYVDGVEVHNAKTKPREEQYQSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH3 | QVQLVQSGAEVKKPGASVKVSCKASGYTFTNYWMHWVRQAPGQ | 315 |
| | GLEWMGEINPIQGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH3_a | QVQLVQSGAEVKKPGASVKVSCKASGYTFTNYWMHWVRQAPGQ | 316 |
| | GLEWMGEINPIQGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH3_b | QVQLVQSGAEVKKPGASVKVSCKASGYTFTNYWMHWVRQAPGQ | 317 |
| | GLEWMGEINPIQGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCGVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH3_c | QVQLVQSGAEVKKPGASVKVSCKASGYTFTNYWMHWVRQAPGQ | 318 |
| | GLEWMGEINPIQGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKARPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH3_d | QVQLVQSGAEVKKPGASVKVSCKASGYTFTNYWMHWVRQAPGQ | 319 |
| | GLEWMGEINPIQGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVIINAKTKPREEQYNSTYRVVSVLTVLHQDWLNG | |
| | KEYKCKVSNKALGAP1EKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH3_e | QVQLVQSGAEVKKPGASVKVSCKASGYTFTNYWMHWVRQAPGQ | 320 |
| | GLEWMGEINPIQGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPELLGGPSVFLFPPKPKDTLMISRTPEVTCVVVAVSHEDPEV | |
| | KFNWYVDGVEVHNAKTKPREEQYGSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH4 | QVQLVQSGAEVKKPGASVKVSCKASGYTFTNYWMHWVRQAPGQ | 321 |
| | GLEWMGEINPINGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPELLGGPSVTLFPPKPKDTLMISRTPEVTCVWDVSHEDPEV | |
| | KFNWYVDGVEVHNAKTKPREEQYNSTYRVVSVLTVLHQDWLNG | |
| | KEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNFIYTQKSLSLSPG | |
| 13E4_VH4_a | QVQLVQSGAEVKKPGASVKVSCKASGYTFTNYWMHWVRQAPGQ | 322 |
| | GLEWMGEINPINGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALQCLVKDYFPEPVTVSWNSGALTSQVHTFPAVLQSSQLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWENG | |
| | KEYKCKVSNKALPAPIEKTISKAKGQPREPQVYTLPPSREEMTKNQ | |
| | VSLTCLVKQFYPSDIAVEWESNGQPENNYKTTPPVLDSDQSFFLYS | |
| | KLTVDKSRWQQGNVFSCSVMHEALHNHYTQKSLSLSPG | |
| 13E4_VH4_b | QVQLVQSGAEVKKPGASVKVSCKASGYTFTNYWMHWVRQAPGQ | 323 |
| | GLEWMGEINPINGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCGVSNKALPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH4_c | QVQLVQSGAEVKKPGASVKVSCKASGYTFINYWMHWVRQAPGQ | 324 |
| | GLEWMGEINPINGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKARPAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH4_d | QVQLVQSGAEVKKPGASVKVSCKASGYTFTNYWMHWVRQAPGQ | 325 |
| | GLEWMGEINPINGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKRVEPKSCDKTHTC | |
| | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNG | |
| | KEYKCKVSNKALGAPIEKTISKAKGQPREPQVYTLPPSREEMTKNQ | |
| | VSLTCLVKGFYPSDIAVEWESNGQPENNYKTTPPVLDSDGSFFLYS | |
| | KLTVDKSRWQQGNVFSCSVMHEALHNHYTQKSLSLSPG | |
| 13E4_VH4_e | QVQLVQSGAEVKKPGASVKVSCKASGYTFTNYWMHWVRQAPGQ | 326 |
| | GLEWMGEINPINGRSNYAEKFQGRVTLTVDTSSSTAYMELSSLRSE | |
| | DTATYYCARGTRAMHYWGQGTLVTVSSASTKGPSVFPLAPSSKST | |
| | SGGTAALGCLVKDYFPEPVTVSWNSGALTSQVHTFPAVLQSSQLY | |
| | SLSSVVTVPSSSLGTQTYICNVNHKPSNTKVDKRVEPKSCDKTHTC | |
| | PPCPAPELLGGPSVFLFPPKPKDTLMISRTPEVTCVVVAVSHEDPEV | |
| | KFNWYVDGVEVHNAKTKPREEQYGSTYRVVSVLTVLHQDWLNG | |
| | KEYKCKVSNKALPAPIEKTISKAKGQPREPQVYTLPPSREEMTKNQ | |
| | VSLTCLVKQFYPSDIAVEWESNGQPENNYKTTPPVLDSDQSFFLYS | |
| | KLTVDKSRWQQGNVFSCSVMHEALHNHYTQKSLSLSPG | |

TABLE 7

| | | SEQ ID |
|---|---|---|
| NAME | LC SEQUENCE | NO: |
| 13E4_VL1 | DIQMTQSPSSLSASVGDRVTITCRTSENIYNNLAWYQQKPGKSPKL | 327 |
| | LIYAATNLADGVPSRFSGSGSGTDYTLTISSLQPEDFATYYCQHFW | |
| | GTPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| 13E4_VL2 | DIQMTQSPSSLSASVGDRVTITCRTSENIYNNLAWYQQKPGKAPKL | 328 |
| | LIYAATNLADGVPSRFSGSGSGTDYTLTISSLQPEDFATYYCQHFW | |
| | GTPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| 13E4_VL3 | DIQMTQSPSSLSASVGDRVTITCRTSENIYNNLAWYQQKPGKAPKL | 329 |
| | LIYAATNLAEGVPSRFSGSQSGTDYTLTISSLQPEDFATYYCQHFW | |
| | GTPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |
| 13E4_VL4 | DIQMTQSPSSLSASVGDRVTITCRTSENIYSNLAWYQQKPGKAPKL | 330 |
| | LIYAGTNLADGVPSRFSGSGSGTDYTLTISSLQPEDFANYYCQHFW | |
| | GTPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKA | |
| | DYEKHKVYACEVTHQGLSSPVTKSFNRGEC | |

In some aspects, an anti-transferrin receptor antibody described herein has an improved serum half-life compared to a reference anti-transferrin receptor antibody. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference anti-transferrin receptor antibody.

In some aspects, the binding moiety A is conjugated to a polynucleic acid molecule (B) non-specifically. In some instances, the binding moiety A is conjugated to a polynucleic acid molecule (B) via a lysine residue or a cysteine residue, in a non-site specific manner. In some instances, the binding moiety A is conjugated to a polynucleic acid molecule (B) via a lysine residue (e.g., lysine residue present in the binding moiety A) in a non-site specific manner. In some cases, the binding moiety A is conjugated to a polynucleic acid molecule (B) via a cysteine residue (e.g., cysteine residue present in the binding moiety A) in a non-site specific manner.

In some aspects, the binding moiety A is conjugated to a polynucleic acid molecule (B) in a site-specific manner. In some instances, the binding moiety A is conjugated to a polynucleic acid molecule (B) through a lysine residue, a cysteine residue, at the 5′-terminus, at the 3′-terminus, an unnatural amino acid, or an enzyme-modified or enzyme-catalyzed residue, via a site-specific manner. In some instances, the binding moiety A is conjugated to a polynucleic acid molecule (B) through a lysine residue (e.g., lysine residue present in the binding moiety A) via a site-specific manner. In some instances, the binding moiety A is conjugated to a polynucleic acid molecule (B) through a cysteine residue (e.g., cysteine residue present in the binding moiety A) via a site-specific manner. In some instances, the binding moiety A is conjugated to a polynucleic acid molecule (B) at the 5′-terminus via a site-specific manner. In some instances, the binding moiety A is conjugated to a polynucleic acid molecule (B) at the 3′-terminus via a site-specific manner. In some instances, the binding moiety A is conjugated to a polynucleic acid molecule (B) through an unnatural amino acid via a site-specific manner. In some instances, the binding moiety A is conjugated to a polynucleic acid molecule (B) through an enzyme-modified or enzyme-catalyzed residue via a site-specific manner.

In some aspects, one or more polynucleic acid molecule (B) is conjugated to a binding moiety A. In some instances, about 1, 2, 3, 4, 5, 6, 7, 8, 9, 10, 11, 12, 13, 14, 15, 16, or more polynucleic acid molecules are conjugated to one binding moiety A. In some instances, about 1 polynucleic acid molecule is conjugated to one binding moiety A. In some instances, about 2 polynucleic acid molecules are conjugated to one binding moiety A. In some instances, about 3 polynucleic acid molecules are conjugated to one binding moiety A. In some instances, about 4 polynucleic acid molecules are conjugated to one binding moiety A. In some instances, about 5 polynucleic acid molecules are conjugated to one binding moiety A. In some instances, about 6 polynucleic acid molecules are conjugated to one binding moiety A. In some instances, about 7 polynucleic acid molecules are conjugated to one binding moiety A. In some instances, about 8 polynucleic acid molecules are conjugated to one binding moiety A. In some instances, about 9 polynucleic acid molecules are conjugated to one binding moiety A. In some instances, about 10 polynucleic acid molecules are conjugated to one binding moiety A. In some instances, about 11 polynucleic acid molecules are conjugated to one binding moiety A. In some instances, about 12 polynucleic acid molecules are conjugated to one binding moiety A. In some instances, about 13 polynucleic acid molecules are conjugated to one binding moiety A. In some instances, about 14 polynucleic acid molecules are conjugated to one binding moiety A. In some instances, about 15 polynucleic acid molecules are conjugated to one binding moiety A. In some instances, about 16 polynucleic acid molecules are conjugated to one binding moiety A. In some cases, the one or more polynucleic acid molecules are the same. In other cases, the one or more polynucleic acid molecules are different.

In some aspects, the number of polynucleic acid molecule (B) conjugated to a binding moiety A forms a ratio. In some instances, the ratio is referred to as a DAR (drug-to-antibody) ratio, in which the drug as referred to herein is the polynucleic acid molecule (B). In some instances, the DAR ratio of the polynucleic acid molecule (B) to binding moiety A is about 1, 2, 3, 4, 5, 6, 7, 8, 9, 10, 11, 12, 13, 14, 15, 16, or greater. In some instances, the DAR ratio of the polynucleic acid molecule (B) to binding moiety A is about 1 or greater. In some instances, the DAR ratio of the polynucleic acid molecule (B) to binding moiety A is about 2 or greater. In some instances, the DAR ratio of the polynucleic acid molecule (B) to binding moiety A is about 3 or greater. In some instances, the DAR ratio of the polynucleic acid molecule (B) to binding moiety A is about 4 or greater. In some instances, the DAR ratio of the polynucleic acid molecule (B) to binding moiety A is about 5 or greater. In some instances, the DAR ratio of the polynucleic acid molecule (B) to binding moiety A is about 6 or greater. In some instances, the DAR ratio of the polynucleic acid molecule (B) to binding moiety A is about 7 or greater. In some instances, the DAR ratio of the polynucleic acid molecule (B) to binding moiety A is about 8 or greater. In some instances, the DAR ratio of the polynucleic acid molecule (B) to binding moiety A is about 9 or greater. In some instances, the DAR ratio of the polynucleic acid molecule (B) to binding moiety A is about 10 or greater. In some instances, the DAR ratio of the polynucleic acid molecule (B) to binding moiety A is about 11 or greater. In some instances, the DAR ratio of the polynucleic acid molecule (B) to binding moiety A is about 12 or greater.

In some instances, the DAR ratio of the polynucleic acid molecule (B) to binding moiety A is about 1, 2, 3, 4, 5, 6, 7, 8, 9, 10, 11, 12, 13, 14, 15, or 16. In some instances, the DAR ratio of the polynucleic acid molecule (B) to binding moiety A is about 1. In some instances, the DAR ratio of the polynucleic acid molecule (B) to binding moiety A is about 2. In some instances, the DAR ratio of the polynucleic acid molecule (B) to binding moiety A is about 3. In some instances, the DAR ratio of the polynucleic acid molecule (B) to binding moiety A is about 4. In some instances, the DAR ratio of the polynucleic acid molecule (B) to binding moiety A is about 5. In some instances, the DAR ratio of the polynucleic acid molecule (B) to binding moiety A is about 6. In some instances, the DAR ratio of the polynucleic acid molecule (B) to binding moiety A is about 7. In some instances, the DAR ratio of the polynucleic acid molecule (B) to binding moiety A is about 8. In some instances, the DAR ratio of the polynucleic acid molecule (B) to binding moiety A is about 9. In some instances, the DAR ratio of the polynucleic acid molecule (B) to binding moiety A is about 10. In some instances, the DAR ratio of the polynucleic acid molecule (B) to binding moiety A is about 11. In some instances, the DAR ratio of the polynucleic acid molecule (B) to binding moiety A is about 12. In some instances, the DAR ratio of the polynucleic acid molecule (B) to binding moiety A is about 13. In some instances, the DAR ratio of the polynucleic acid molecule (B) to binding moiety A is about 14.

In some instances, the DAR ratio of the polynucleic acid molecule (B) to binding moiety A is about 15. In some instances, the DAR ratio of the polynucleic acid molecule (B) to binding moiety A is about 16.

In some instances, the DAR ratio of the polynucleic acid molecule (B) to binding moiety A is 1, 2, 3, 4, 5, 6, 7, 8, 9, 10, 11, 12, 13, 14, 15, or 16. In some instances, the DAR ratio of the polynucleic acid molecule (B) to binding moiety A is 1. In some instances, the DAR ratio of the polynucleic acid molecule (B) to binding moiety A is 2. In some instances, the DAR ratio of the polynucleic acid molecule (B) to binding moiety A is 4. In some instances, the DAR ratio of the polynucleic acid molecule (B) to binding moiety A is 6. In some instances, the DAR ratio of the polynucleic acid molecule (B) to binding moiety A is 8. In some instances, the DAR ratio of the polynucleic acid molecule (B) to binding moiety A is 12.

In some instances, a conjugate comprising polynucleic acid molecule (B) and binding moiety A has improved activity as compared to a conjugate comprising polynucleic acid molecule (B) without a binding moiety A. In some instances, improved activity results in enhanced biologically relevant functions, e.g., improved stability, affinity, binding, functional activity, and efficacy in treatment or prevention of a disease state. In some instances, the disease state is a result of one or more mutated exons of a gene. In some instances, the conjugate comprising polynucleic acid molecule (B) and binding moiety A results in increased exon skipping of the one or more mutated exons as compared to the conjugate comprising polynucleic acid molecule (B) without a binding moiety A. In some instances, exon skipping is increased by at least or about 5%, 10%,20%, 25%, 30%, 40%, 50%, 60%, 70%, 80%, 90%, 95%, or more than 95% in the conjugate comprising polynucleic acid molecule (B) and binding moiety A as compared to the conjugate comprising polynucleic acid molecule (B) without a binding moiety A.

In some aspects, an antibody or antigen binding fragment is further modified using conventional techniques known in the art, for example, by using amino acid deletion, insertion, substitution, addition, and/or by recombination and/or any other modification (e.g., posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No.

WO97/34631, which discloses amino acid residues involved in the interaction between the Fc domain and the FcRn receptor. Methods for introducing such modifications in the nucleic acid sequence underlying the amino acid sequence of an antibody or antigen binding fragment is well known to the person skilled in the art.

In some instances, an antigen binding fragment further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such aspects, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some aspects, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its antigen binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further aspects, the binding is exemplified by the specificity of a “key-lock-principle”. Thusin some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or antigen binding fragment or a reduced off-target effect. For example, the antibody or antigen binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Additional Binding Moieties

In some aspects, the binding moiety is a plasma protein. In some instances, the plasma protein comprises albumin. In some instances, the binding moiety A is albumin. In some instances, albumin is conjugated by one or more of a conjugation chemistry described herein to a polynucleic acid molecule. In some instances, albumin is conjugated by native ligation chemistry to a polynucleic acid molecule. In some instances, albumin is conjugated by lysine conjugation to a polynucleic acid molecule.

In some instances, the binding moiety is a steroid. Exemplary steroids include cholesterol, phospholipids, di-andtriacylglycerols, fatty acids, hydrocarbons that are saturated, unsaturated, comprise substitutions, or combinations thereof. In some instances, the steroid is cholesterol. In some instances, the binding moiety is cholesterol. In some instances, cholesterol is conjugated by one or more of a conjugation chemistry described herein to a polynucleic acid molecule. In some instances, cholesterol is conjugated by native ligation chemistry to a polynucleic acid molecule. In some instances, cholesterol is conjugated by lysine conjugation to a polynucleic acid molecule.

In some instances, the binding moiety is a polymer, including but not limited to polynucleic acid molecule aptamers that bind to specific surface markers on cells. In this instance the binding moiety is a polynucleic acid that does not hybridize to a target gene or mRNA, but instead is capable of selectively binding to a cell surface marker similarly to an antibody binding to its specific epitope of a cell surface marker.

In some cases, the binding moiety is a peptide. In some cases, the peptide comprises between about 1 and about 3 kDa. In some cases, the peptide comprises between about 1.2 and about 2.8 kDa, about 1.5 and about 2.5 kDa, or about 1.5 and about 2 kDa. In some instances, the peptide is a bicyclic peptide. In some cases, the bicyclic peptide is a constrained bicyclic peptide. In some instances, the binding moiety is a bicyclic peptide (e.g., bicycles from Bicycle Therapeutics).

In additional cases, the binding moiety is a small molecule. In some instances, the small molecule is an antibody-recruiting small molecule. In some cases, the antibody-recruiting small molecule comprises a target-binding terminus and an antibody-binding terminus, in which the target-binding terminus is capable of recognizing and interacting with a cell surface receptor.

For example, in some instances, the target-binding terminus comprising a glutamate urea compound enables interaction with PSMA, thereby, enhances an antibody interaction with a cell that expresses PSMA. In some instances, a binding moiety is a small molecule described in Zhang et al., “A remote arene-binding site on prostate specific membrane antigen revealed by antibody-recruiting small molecules,” J Am Chem Soc. 132(36): 12711-12716 (2010); or McEnaney, et al., “Antibody-recruiting molecules: an emerging paradigm for engaging immune function in treating human disease,” ACS Chem Biol. 7(7): 1139-1151 (2012).

### Production of Antibodies or Antigen Binding Fragment Thereof

In some aspects, polypeptides described herein (e.g., antibodies and antigen binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or antigen binding fragment thereof is expressed recombinantly, and the nucleic acid encoding the antibody or antigen binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its antigen binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in MonoclonalAntibodies and Cancer Therapy, Alan R. Liss, Inc., pp. 77-96).

Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad.

Sci. USA 94:4937).

In some aspects, techniques developed for the production of “chimeric antibodies” (Morrison et al., 1984, Proc. Natd. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some aspects, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some aspects, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue, specific promoter.

In some aspects, a variety of host-expression vector systems is utilized to express an antibody or its antigen binding fragment described herein. Such host-expression systems represent vehicles by which the coding sequences of the antibody is produced and subsequently purified, but also represent cells that are, when transformed or transfected with the appropriate nucleotide coding sequences, express an antibody or its antigen binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its antigen binding fragment coding sequences; yeast (e.g., Saccharomyces Pichia) transformed with recombinant yeast expression vectors containing an antibody or its antigen binding fragment coding sequences; insect cell systems infected with recombinant virus expression vectors (e.g., baculovirus) containing an antibody or its antigen binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its antigen binding fragment coding sequences; or mammalian cell systems (e.g., COS, CHO, BH, 293, 293 T, 3 T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its antigen binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell22:817) genes are employed in tk-, hgprt- or aprt- cells, respectively. Also, antimetabolite resistance are used as the basis of selection for the following genes: DHFR, which confers resistance to methotrexate (Wigler et al., 1980, Proc. Natl. Acad. Sci. USA 77:357; O'Hare et al., 1981, Proc. Natl. Acad. Sci. USA 78:1527); GPT, which confers resistance to mycophenolic acid (Mulligan & Berg, 1981, Proc. Natl. Acad. Sci. USA 78:2072); neo, which confers resistance to the aminoglycoside G-418 (ClinicalPharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification or analysis of an antibody or antibody conjugates is used, for example, by chromatography (e.g., ion exchange, affinity, particularly by affinity for the specific antigen after Protein A, and sizing column chromatography), centrifugation, differential solubility, or by any other standard technique for the purification of proteins. Exemplary chromatography methods included, but are not limited to, strong anion exchange chromatography, hydrophobic interaction chromatography, size exclusion chromatography, and fast protein liquid chromatography.

### Conjugation Chemistry

In some aspects, a polynucleic acid molecule B is conjugated to a binding moiety. In some aspects, a polynucleic acid molecule B is conjugated to a binding moiety in a formula A-X-B (X is a linker conjugating A and B). In some instances, the binding moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances.

Additional examples of binding moiety also include steroids, such as cholesterol, phospholipids, di-and triacylglycerols, fatty acids, hydrocarbons (e.g., saturated, unsaturated, or contains substitutions), enzyme substrates, biotin, digoxigenin, and polysaccharides. In some instances, the binding moiety is an antibody or antigen binding fragment thereof. In some instances, the polynucleic acid molecule is further conjugated to a polymer, and optionally an endosomolytic moiety.

In some aspects, the polynucleic acid molecule is conjugated to the binding moiety by a chemical ligation process. In some instances, the polynucleic acid molecule is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat.No. 8,936,910.

In some aspects, the polynucleic acid molecule is conjugated to the binding moiety either site-specifically or non-specifically via native ligation chemistry.

In some instances, the polynucleic acid molecule is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the polynucleic acid molecule is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates usingunnatural amino acids,” PNAS 109(40): 16101-16106 (2012)).

In some instances, the polynucleic acid molecule is conjugated to the binding moiety by a site-directed method utilizing an enzyme-catalyzed process. In some instances, the site-directed method utilizes SMARTag^{T}M technology (Catalent, Inc.). In some instances, the SMARTag^{T}M technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification ofrecombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the polynucleic acid molecule is conjugated to the binding moiety utilizing a microbial transglutaminase-catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the polynucleic acid molecule is conjugated to the binding moiety by a method as described in PCT Publication No. WO2014/140317, which utilizes a sequence-specific transpeptidase.

In some instances, the polynucleic acid molecule is conjugated to the binding moiety by a method as described in U.S. Patent Publication Nos. 2015/0105539 and 2015/0105540.

### Polymer Conjugating Moiety

In some aspects, a polymer moiety C is further conjugated to a polynucleic acid molecule described herein, a binding moiety described herein, or in combinations thereof. In some instances, a polymer moiety C is conjugated a polynucleic acid molecule in a formula A-X₁-B—X₂—C(X₁, X₂ as two linkers conjugating A and B, B and C, respectively). In some cases, a polymer moiety C is conjugated to a binding moiety. In other cases, a polymer moiety C is conjugated to a polynucleic acid molecule-binding moiety molecule. In additional cases, a polymer moiety C is conjugated, as illustrated supra.

In some instances, the polymer moiety C is a natural or synthetic polymer, consisting of long chains of branched or unbranched monomers, and/or cross-linked network of monomers in two or three dimensions. In some instances, the polymer moiety C includes a polysaccharide, lignin, rubber, or polyalkylen oxide (e.g., polyethylene glycol). In some instances, the at least one polymer moiety C includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer moiety C comprises polyalkylene oxide. In some instances, the polymer moiety C comprises PEG. In some instances, the polymer moiety C comprises polyethylene imide (PEI) or hydroxy ethyl starch (HES).

In some instances, C is a PEG moiety. In some instances, the PEG moiety is conjugated at the 5′ terminus of the polynucleic acid molecule while the binding moiety is conjugated at the 3′ terminus of the polynucleic acid molecule. In some instances, the PEG moiety is conjugated at the 3′ terminus of the polynucleic acid molecule while the binding moiety is conjugated at the 5′ terminus of the polynucleic acid molecule. In some instances, the PEG moiety is conjugated to an internal site of the polynucleic acid molecule. In some instances, the PEG moiety, the binding moiety, or a combination thereof, are conjugated to an internal site of the polynucleic acid molecule. In some instances, the conjugation is a direct conjugation. In some instances, the conjugation is via native ligation.

In some aspects, the polyalkylene oxide (e.g., PEG) is a polydisperse or monodisperse compound. In some instances, polydisperse material comprises disperse distribution of different molecular weight of the material, characterized by mean weight (weight average) size and dispersity. In some instances, the monodisperse PEG comprises one size of molecules. In some aspects, C is poly- or monodispersed polyalkylene oxide (e.g., PEG) and the indicated molecular weight represents an average of the molecular weight of the polyalkylene oxide, e.g., PEG, molecules.

In some aspects, the molecular weight of the polyalkylene oxide (e.g., PEG) is about 200, 300,400, 500,600,700,800,900, 1000, 1100,1200,1300,1400, 1450,1500, 1600,1700,1800, 1900, 2000,2100, 2200,2300, 2400,2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some aspects, C is polyalkylene oxide (e.g., PEG) and has a molecular weight of about 200, 300, 400,500,600,700,800,900,1000,1100,1200,1300,1400, 1450,1500, 1600, 1700, 1800, 1900,2000,2100,2200,2300,2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some aspects, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900,3000, 3250,3350, 3500,3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,0001 Da.

In some instances, the molecular weight of C is about 200 Da. In some instances, the molecular weight of C is about 300 Da. In some instances, the molecular weight of C is about 400 Da. In some instances, the molecular weight of C is about 500 Da. In some instances, the molecular weight of C is about 600 Da. In some instances, the molecular weight of C is about 700 Da. In some instances, the molecular weight of C is about 800 Da. In some instances, the molecular weight of C is about 900 Da. In some instances, the molecular weight of C is about 1000 Da. In some instances, the molecularweight of C is about 1100 Da. In some instances, the molecular weight of C is about 1200 Da. In some instances, the molecular weight of C is about 1300 Da. In some instances, the molecular weight of C is about 1400 Da. In some instances, the molecular weight of C is about 1450 Da. In some instances, the molecular weight of C is about 1500 Da. In some instances, the molecular weight of C is about 1600 Da. In some instances, the molecular weight of C is about 1700 Da. In some instances, the molecular weight of C is about 1800 Da. In some instances, the molecular weight of C is about 1900 Da. In some instances, the molecular weight of C is about 2000 Da. In some instances, the molecular weight of C is about 2100 Da. In some instances, the molecular weight of C is about 2200 Da. In some instances, the molecular weight of C is about 2300 Da. In some instances, the molecular weight of C is about 2400 Da. In some instances, the molecular weight of C is about 2500 Da. In some instances, the molecular weight of C is about 2600 Da. In some instances, the molecular weight of C is about 2700 Da. In some instances, the molecular weight of C is about 2800 Da. In some instances, the molecular weight of C is about 2900 Da. In some instances, the molecular weight of C is about 3000 Da. In some instances, the molecular weight of C is about 3250 Da. In some instances, the molecular weight of C is about 3350 Da. In some instances, the molecular weight of C is about 3500 Da. In some instances, the molecular weight of C is about 3750 Da. In some instances, the molecular weight of C is about 4000 Da. In some instances, the molecular weight of C is about 4250 Da. In some instances, the molecular weight of C is about 4500 Da. In some instances, the molecular weight of C is about 4600 Da. In some instances, the molecular weight of C is about 4750 Da. In some instances, the molecular weight of C is about 5000 Da. In some instances, the molecular weight of C is about 5500 Da. In some instances, the molecular weight of C is about 6000 Da. In some instances, the molecular weight of C is about 6500 Da. In some instances, the molecular weight of C is about 7000 Da. In some instances, the molecular weight of C is about 7500 Da. In some instances, the molecular weight of C is about 8000 Da. In some instances, the molecular weight of C is about 10,000 Da. In some instances, the molecular weight of C is about 12,000 Da. In some instances, the molecular weight of C is about 20,000 Da. In some instances, the molecular weight of C is about 35,000 Da. In some instances, the molecular weight of C is about 40,000 Da. In some instances, the molecular weight of C is about 50,000 Da. In some instances, the molecular weight of C is about 60,000 Da. In some instances, the molecular weight of C is about 100,000 Da.

In some aspects, the polyalkylene oxide (e.g., PEG) comprises discrete ethylene oxide units (e.g., four to about 48 ethylene oxide units). In some instances, the polyalkylene oxide comprising the discrete ethylene oxide units is a linear chain. In other cases, the polyalkylene oxide comprising the discrete ethylene oxide units is a branched chain.

In some instances, the polymer moiety C is a polyalkylene oxide (e.g., PEG) comprising discrete ethylene oxide units. In some cases, the polymer moiety C comprises between about 4 and about 48 ethylene oxide units. In some cases, the polymer moiety C comprises about 4, about 5,about 6,about 7, about 8, about 9, about 10, about 11, about 12, about 13, about 14, about 15, about 16, about 17, about 18, about 19, about 20, about 21, about 22, about 23, about 24,about 25,about 26, about 27, about 28, about 29, about 30, about 31, about 32, about 33, about 34, about 35, about 36, about 37, about 38, about 39, about 40, about 41, about 42, about 43, about 44, about 45, about 46, about 47, or about 48 ethylene oxide units.

In some instances, the polymer moiety C is a discrete PEG comprising, e.g., between about 4 and about 48 ethylene oxide units. In some cases, the polymer moiety C is a discrete PEG comprising, e.g., about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, or about 48 ethylene oxide units. In some cases, the polymer moiety C is a discrete PEG comprising, e.g., about 4 ethylene oxide units. In some cases, the polymer moiety C is a discrete PEG comprising, e.g., about 5 ethylene oxide units. In some cases, the polymer moiety C is a discrete PEG comprising, e.g., about 6 ethylene oxide units. In some cases, the polymer moiety C is a discrete PEG comprising, e.g., about 7 ethylene oxide units. In some cases, the polymer moiety C is a discrete PEG comprising, e.g., about 8 ethylene oxide units. In some cases, the polymer moiety C is a discrete PEG comprising, e.g., about 9 ethylene oxide units. In some cases, the polymer moiety C is a discrete PEG comprising, e.g., about 10 ethylene oxideunits. In some cases, the polymer moiety C is a discrete PEG comprising, e.g., about 11 ethylene oxide units. In some cases, the polymer moiety C is a discrete PEG comprising, e.g., about 12 ethylene oxide units. In some cases, the polymer moiety C is a discrete PEG comprising, e.g., about 13 ethylene oxide units. In some cases, the polymer moiety C is a discrete PEG comprising, e.g., about 14 ethylene oxide units. In some cases, the polymer moiety C is a discrete PEG comprising, e.g., about 15 ethylene oxide units. In some cases, the polymer moiety C is a discrete PEG comprising, e.g., about 16 ethylene oxide units. In some cases, the polymer moiety C is a discrete PEG comprising, e.g., about 17 ethylene oxide units. In some cases, the polymer moiety C is a discrete PEG comprising, e.g., about 18 ethylene oxide units. In some cases, the polymer moiety C is a discrete PEG comprising, e.g., about 19 ethylene oxide units. In some cases, the polymer moiety C is a discrete PEG comprising, e.g., about 20 ethylene oxideunits. In some cases, the polymer moiety C is a discrete PEG comprising, e.g., about 21 ethylene oxide units. In some cases, the polymer moiety C is a discrete PEG comprising, e.g., about 22 ethylene oxide units. In some cases, the polymer moiety C is a discrete PEG comprising, e.g., about 23 ethylene oxide units. In some cases, the polymer moiety C is a discrete PEG comprising, e.g., about 24 ethylene oxide units. In some cases, the polymer moiety C is a discrete PEG comprising, e.g., about 25 ethylene oxide units. In some cases, the polymer moiety C is a discrete PEG comprising, e.g., about 26 ethylene oxide units. In some cases, the polymer moiety C is a discrete PEG comprising, e.g., about 27 ethylene oxide units. In some cases, the polymer moiety C is a discrete PEG comprising, e.g., about 28 ethylene oxide units. In some cases, the polymer moiety C is a discrete PEG comprising, e.g., about 29 ethylene oxide units. In some cases, the polymer moiety C is a discrete PEG comprising, e.g., about 30 ethylene oxideunits. In some cases, the polymer moiety C is a discrete PEG comprising, e.g., about 31 ethylene oxide units. In some cases, the polymer moiety C is a discrete PEG comprising, e.g., about 32 ethylene oxide units. In some cases, the polymer moiety C is a discrete PEG comprising, e.g., about 33 ethylene oxide units. In some cases, the polymer moiety C is a discrete PEG comprising, e.g., about 34 ethylene oxide units. In some cases, the polymer moiety C is a discrete PEG comprising, e.g., about 35 ethylene oxide units. In some cases, the polymer moiety C is a discrete PEG comprising, e.g., about 36 ethylene oxide units. In some cases, the polymer moiety C is a discrete PEG comprising, e.g., about 37 ethylene oxide units. In some cases, the polymer moiety C is a discrete PEG comprising, e.g., about 38 ethylene oxide units. In some cases, the polymer moiety C is a discrete PEG comprising, e.g., about 39 ethylene oxide units. In some cases, the polymer moiety C is a discrete PEG comprising, e.g., about 40 ethylene oxideunits. In some cases, the polymer moiety C is a discrete PEG comprising, e.g., about 41 ethylene oxide units. In some cases, the polymer moiety C is a discrete PEG comprising, e.g., about 42 ethylene oxide units. In some cases, the polymer moiety C is a discrete PEG comprising, e.g., about 43 ethylene oxide units. In some cases, the polymer moiety C is a discrete PEG comprising, e.g., about 44 ethylene oxide units. In some cases, the polymer moiety C is a discrete PEG comprising, e.g., about 45 ethylene oxide units. In some cases, the polymer moiety C is a discrete PEG comprising, e.g., about 46 ethylene oxide units. In some cases, the polymer moiety C is a discrete PEG comprising, e.g., about 47 ethylene oxide units. In some cases, the polymer moiety C is a discrete PEG comprising, e.g., about 48 ethylene oxide units.

In some cases, the polymer moiety C is dPEG® (Quanta Biodesign Ltd).

In some aspects, the polymer moiety C comprises a cationic mucic acid-based polymer (cMAP). In some instances, cMAP comprises one or more subunit of at least one repeating subunit, and the subunit structure is represented as Formula (V):

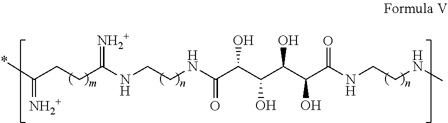

wherein m is independently at each occurrence 1, 2, 3, 4, 5, 6, 7, 8, 9, or 10, preferably 4-6 or 5; and n is independently at each occurrence 1, 2, 3, 4, or 5. In some aspects, m and n are, for example, about 10.

In some instances, cMAP is further conjugated to a PEG moiety, generating a cMAP-PEG copolymer, an mPEG-cMAP-PEGm triblock polymer, or a cMAP-PEG-cMAP triblock polymer. In some instances, the PEG moiety is in a range of from about 500 Da to about 50,000 Da. In some instances, the PEG moiety is in a range of from about 500 Da to about 1000 Da, greater than 1000 Da to about 5000 Da, greater than 5000 Da to about 10,000 Da, greater than 10,000 to about 25,000 Da, greater than 25,000 Da to about 50,000 Da, or any combination of two or more of these ranges.

In some instances, the polymer moiety C is cMAP-PEG copolymer, an mPEG-cMAP-PEGm triblock polymer, or a cMAP-PEG-cMAP triblock polymer. In some cases, the polymer moiety C is cMAP-PEG copolymer. In other cases, the polymer moiety C is an mPEG-cMAP-PEGm triblock polymer. In additional cases, the polymer moiety C is a cMAP-PEG-cMAP triblock polymer.

In some aspects, the polymer moiety C is conjugated to the polynucleic acid molecule, the binding moiety, and optionally to the endosomolytic moiety as illustrated supra.

### Endosomolytic or Cell Membrane Penetration Moiety

In some aspects, a molecule of Formula (I): A-X₁-B-X₂-C, further comprises an additional conjugating moiety. In some instances, the additional conjugating moiety is an endosomolytic moiety and/or a cell membrane penetration moiety. In some cases, the endosomolytic moiety is a cellular compartmental release component, such as a compound capable of releasing from any of the cellular compartments known in the art, such as the endosome, lysosome, endoplasmic reticulum (ER), Golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule. In some cases, the endosomolytic moiety comprises an endosomolytic polypeptide. In other cases, the endosomolytic moiety comprises an endosomolytic polymer. In some cases, the cell membrane penetration moiety comprises a cell penetrating peptide (CPP). In other cases, the cell membrane penetration moiety comprises a cell penetrating lipid. In other cases, the cell membrane penetration moiety comprises a cell penetrating small molecule.

### Endosomolytic and Cell Membrane Penetration Polypeptides

In some aspects, a molecule of Formula (I): A-X₁-B-X₂-C, is further conjugated with an endosomolytic polypeptide. In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof In additional cases, the endosomolytic polypeptide comprises meucin or its derivatives thereof.

In some instances, INF7 is a 24 residue polypeptide those sequence comprises CGIFGEIEELIEEGLENLIDWGNA (SEQ ID NO: 331), or GLFEAIEGFIENGWEGMIDGWYGC (SEQ ID NO: 332). In some instances, INF7 or its derivatives comprise a sequence of: GLFEAIEGFIENGWEGMIWDYGSGSCG (SEQ ID NO: 333), GLFEAIEGFIENGWEGMIDG WYG-(PEG)6-NH2 (SEQ ID NO: 334), or GLFEAIEGFIENGWEGMIWDYG-SGSC-K(GalNAc)2 (SEQ ID NO: 335).

In some cases, melittin is a 26 residue polypeptide those sequence comprises CLIGAILKVLATGLPTLISWIKNKRKQ (SEQ ID NO: 336), or GIlGAVLKVLTTGLPALISWIKRKRQQ (SEQ ID NO: 337). In some instances, melittin comprises a polypeptide sequence as described in U.S. Pat. No. 8,501,930.

In some instances, meucin is an antimicrobial peptide (AMP) derived from the venom gland of the scorpion Mesobuthus eupeus. In some instances, meucin comprises of meucin-13 those sequence comprises IFGAIAGLLKNIF-NH₂ (SEQ ID NO: 338) and meucin-18 those sequence comprises FFGHLFKLATKIIPSLFQ (SEQ ID NO: 339).

In some instances, the endosomolytic polypeptide comprises a polypeptide in which its sequence is at least 50%, 60%, 70%, 80%, 90%, 95%, or 99% sequence identity to INF7 or its derivatives thereof, melittin or its derivatives thereof, or meucin or its derivatives thereof. In some instances, the endosomolytic moiety comprises INF7 or its derivatives thereof, melittin or its derivatives thereof, or meucin or its derivatives thereof.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 331-335. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 331. In some cases, the endosomolytic moiety comprises a polypeptide having at least 50^{%}, 55%, 60%, 65%, 70%, 75%, 80%, 85%, 90, 95% 96%, 97%, 98%, 99%, or 100% sequence identity to SEQ ID NOs: 332-335. In some cases, the endosomolytic moiety comprises SEQ ID NO: 331. In some cases, the endosomolytic moiety comprises SEQ ID NOs: 332-335. In some cases, the endosomolytic moiety consists of SEQ ID NO: 331. In some cases, the endosomolytic moiety consists of SEQ ID NOs: 332-335.

In some instances, the endosomolytic moiety is melittin or its derivatives thereof. In some cases, the endosomolytic moiety comprises a polypeptide having at least 50%, 55%, 60%, 65%, 70%, 75%, 80, 85, 90%, 95%, 96%, 97%, 98%, 99%, or 100% sequence identity to SEQ ID NOs: 336 or 337. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 336. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 337. In some cases, the endosomolytic moiety comprises SEQ ID NO: 286. In some cases, the endosomolytic moiety comprises SEQ ID NO: 337. In some cases, the endosomolytic moiety consists of SEQ ID NO: 336. In some cases, the endosomolytic moiety consists of SEQ ID NO: 337.

In some instances, the endosomolytic moiety is meucin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 338 or 339. In some cases, the endosomolytic moiety comprises a polypeptide having at least 50%>,55%,60^{%}>,65%,70^{%}>,75%,80^{%}>,85%,90^{%}>,95%,96%,>97%,>98%, 99%, or 100% sequence identity to SEQ ID NO: 338. In some cases, the endosomolytic moiety comprises a polypeptide having at least 50%, 55%, 60%, 65%, 70%, 75%, 80%, 85%, 90, 95% 96%, 97%, 98%, 99%, or 100% sequence identity to SEQ ID NO: 339. In some cases, the endosomolytic moiety comprises SEQ ID NO: 338. In some cases, the endosomolytic moiety comprises SEQ ID NO: 339. In some cases, the endosomolytic moiety consists of SEQ ID NO: 338. In some cases, the endosomolytic moiety consists of SEQ ID NO: 339. In some instances, the endosomolytic moiety comprises a sequence as illustrated in Table 8.

TABLE 8

| | | | SEQ ID | |
|---|---|---|---|---|
| NAME | ORIGIN | AMINO ACID SEQUENCE | NO: | TYPE |
| Pep-1 | NLS from Simian Virus | KETWWETWWTEWSQPKKK | 340 | Primary |
| | 40 large antigen and | RKV | | amphipathic |
| | Reverse transcriptase of | | | |
| | HIV | | | |
| pVEC | VE-cadherin | LLIILRRRRIRKQAHAHSK | 341 | Primary |
| | | | | amphipathic |
| VT5 | Synthetic peptide | DPKGDPKGVTVTVTVTVTG | 342 | β-sheet |
| | | KGDPKPD | | amphipathic |
| C105Y | 1-antitrypsin | CSIPPEVKFNKPFVYLI | 343 | — |
| Transportan | Galanin and mastoparan | GWTLNSAGYLLGKINLKAL | 344 | Primary |
| | | AALAKKIL | | amphipathic |
| TP10 | Galanin and mastoparan | AGYLLGKINLKALAALAKKI | 345 | Primary |
| | | L | | amphipathic |
| MPG | A hydrophobic domain | GALFLGFLGAAGSTMGA | 346 | β-sheet |
| | from the fusion | | | amphipathic |
| | sequence of HIV gp41 | | | |
| | and NLS of SV40 T | | | |
| | antigen | | | |
| gH625 | Glycoprotein gH of | HGLASTLTRWAHYNALIRA | 347 | Second ary |
| | HSV type 1 | F | | amphipathic |
| | | | | α-helical |
| CADY | PPTG1 peptide | GLWRALWRLLRSLWRLLW | 348 | Secondary |
| | | RA | | amphipathic |
| | | | | α-helical |
| GALA | Synthetic peptide | WEAALAEALAEALAEHLAE | 349 | Secondary |
| | | ALAEALEALAA | | amphipathic |
| | | | | α-helical |
| INF | Influenza HA2 fusion | GLFEAIEGFIENGWEGMIDG | 350 | Secondary |
| | peptide | WYGC | | amphipathic |
| | | | | α-helical/pH- |
| | | | | dependent |
| | | | | membrane |
| | | | | active peptide |
| HA2E5- | Influenza HA2 subunit | GLFGAIAGFTENGWEGMIDG | 351 | Secondary |
| TAT | of influenza virus X31 | WYG | | amphipathic |
| | strain fusion peptide | | | α-helical/pH- |
| | | | | dependent |
| | | | | membrane |
| | | | | active peptide |
| HA2- | Influenza HA2 subunit | GLFGAIAGFTENGWEGMIDG | 352 | pH-dependent |
| penetratin | of influenza virus X31 | RQIKIWFQNRRMKW | | membrane |
| | strain fusion peptide | KK-amide | | active peptide |
| HA-K4 | Influenza HA2 subunit | GLFGAIAGFTENGWEGMIDG- | 353 | pH-dependent |
| | of influenza virus X31 | SSKKKK | | membrane |
| | strain fusion peptide | | | active peptide |
| HA2E4 | Influenza HA2 subunit | GLFEAIAGFIENGWEGMIDG | 354 | pH-dependent |
| | of influenza virus X31 | GGYC | | membrane |
| | strain fusion peptide | | | active peptide |
| H5WYG | HA2 analogue | GLFHAIAHFIHGGWH | 355 | pH-dependent |
| | | GLIHGWYG | | membrane |
| | | | | active peptide |
| GALA- | INF3 fusion peptide | GLFEAIEGFIENGWEGLAEA | 356 | pH-dependent |
| INF3- | | LAEALEALAA- | | membrane |
| (PEG)6-NH | | (PEG)6-NH2 | | active peptide |
| CM18- | Cecropin-A-Melittin₂₋₁₂ | KWKLFKKIGAVLKVLTTG- | 357 | pH-dependent |
| TAT11 | (CM₁₈) fusion peptide | YGRKKRRQRRR | | membrane |
| | | | | active peptide |

In some cases, the endosomolytic moiety comprises a Bak BH3 polypeptide which induces apoptosis through antagonization of suppressor targets such as Bcl-2 and/or Bcl-x_{L}. In some instances, the endosomolytic moiety comprises a Bak BH3 polypeptide described in Albarran, etal., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a cell-penetrating polypeptide) as described in PCT Publication Nos. WO2013/166155 or WO2015/069587.

### Endosomolytic Lipids

In some aspects, the endosomolytic moiety is a lipid (e.g., a fusogenic lipid). In some aspects, a molecule of Formula (I): A-X₁-B- X₂-C, is further conjugated with an endosomolytic lipid (e.g., fusogenic lipid). Exemplary fusogenic lipids include 1,2-dileoyl-sn-3-phosphoethanolamine (DOPE), phosphatidylethanolamine (POPE), palmitoyloleoylphosphatidylcholine (POPC), (6Z,9Z,28Z,3 1Z)-heptatriaconta-6,9,28,3 1-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (XTC).

In some instances, an endosomolytic moiety is a lipid (e.g., a fusogenic lipid) described in PCT Publication No. WO09/126,933.

### Endosomolytic Small Molecules

In some aspects, the endosomolytic moiety is a small molecule. In some aspects, a molecule of Formula (I): A-X₁-B- X₂-C, is further conjugated with an endosomolytic small molecule. Exemplary small molecules suitable as endosomolytic moieties include, but are not limited to, quinine, chloroquine, hydroxychloroquines, amodiaquins (carnoquines), amopyroquines, primaquines, mefloquines, nivaquines, halofantrines, quinone imines, or a combination thereof. In some instances, quinoline endosomolytic moieties include, but are not limited to, 7-chloro-4-(4-diethylamino-1-methylbutyl-amino)quinoline (chloroquine); 7-chloro-4-(4-ethyl-(2-hydroxyethyl)-amino-1-methylbutyl-amino)quinoline (hydroxychloroquine); 7-fluoro-4-(4-diethylamino-1-methylbutyl-amino)quinoline; 4-(4-diethylamino-1-methylbutylamino) quinoline; 7-hydroxy-4-(4-diethyl-amino-1-methylbutylamino)quinoline; 7-chloro-4-(4-diethylamino-1-butylamino)quinoline (desmethylchloroquine); 7-fluoro-4-(4-diethylamino-1-butylamino)quinoline); 4-(4-diethyl-amino-1-butylamino)quinoline; 7-hydroxy-4-(4-diethylamino-1-butylamino)quinoline; 7-chloro-4-(1-carboxy-4-diethylamino-1-butylamino)quinoline; 7-fluoro-4-(1-carboxy-4-diethyl-amino-1-butylamino)quinoline; 4-(1-carboxy-4-diethylamino-1-butylamino) quinoline; 7-hydroxy-4-(1-carboxy-4-diethylamino-1-butylamino)quinoline; 7-chloro-4-(1-carboxy-4-diethylamino-1-methylbutylamino)quinoline; 7-fluoro-4-(1-carboxy-4-diethyl-amino-1-methylbutylamino)quinoline; 4-(1-carboxy-4-diethylamino-1-methylbutylamino)quinoline; 7-hydroxy-4-(1-carboxy-4-diethylamino-1-methylbutylamino)quinoline; 7-fluoro-4-(4-ethyl-(2-hydroxyethyl)-amino-1-methylbutylamino)quinoline; 4-(4-ethyl-(2-hydroxy-ethyl)-amino-1-methylbutylamino-)quinoline; 7-hydroxy-4-(4-ethyl-(2-hydroxyethyl)-amino-1-methylbutylamino)quinoline; hydroxychloroquine phosphate; 7-chloro-4-(4-ethyl-(2-hydroxyethyl-1)-amino−1-butylamino)quinoline (desmethylhydroxychloroquine); 7-fluoro-4-(4-ethyl-(2-hydroxyethyl)-amino−1-butylamino)quinoline; 4-(4-ethyl-(2-hydroxyethyl)-amino-1-butylamino)quinoline; 7-hy droxy-4-(4-ethyl-(2-hydroxyethyl)-amino-1-butylamino) quinoline; 7-chloro-4-(1-carboxy-4-ethyl-(2-hydroxyethyl)-amino-1-butylamino)quinoline; 7-fluoro-4-(1-carboxy-4-ethyl-(2-hydroxyethyl)-amino-1-butylamino)quinoline; 4-(1-carboxy-4-ethyl-(2-hydroxyethyl)-amino-1-butylamino)quinoline; 7-hydroxy-4-(1-carboxy-4-ethyl-(2-hydroxyethyl)-amino-1-butylamino)quinoline; 7-chloro-4-(1-carboxy-4-ethyl-(2-hydroxyethyl)-amino-1-methylbutylamino)quinoline; 7-fluoro-4-(1-carboxy-4-ethyl-(2-hydroxyethyl)-amino-1-methylbutylamino)quinoline; 4-(1-carboxy-4-ethyl-(2-hydroxyethyl)-amino-1-methylbutylamino)quinoline; 7-hydroxy-4-(1-carboxy-4-ethyl-(2-hydroxyethyl)-amino-1-methylbutylamino)quinoline; 8-[(4-aminopentyl)amino-6-methoxydihydrochloride quinoline; 1-acetyl-1,2,3,4-tetrahydroquinoline; 8-[(4-aminopentyl)amino]-6-methoxyquinoline dihydrochloride; 1-butyryl-1,2,3,4-tetrahydroquinoline; 3-chloro-4-(4-hydroxy-alpha,alpha′-bis(2-methyl-1-pyrrolidinyl)-2,5-xylidinoquinoline, 4-[(4-diethyl-amino)-1-methylbutyl-amino]-6-methoxyquinoline; 3-fluoro-4-(4-hydroxy-alpha,alpha′-bis(2-methyl-1-pyrrolidinyl)-2,5-xylidinoquinoline, 4-[(4-diethylamino)-1-methylbutyl-amino]-6-methoxyquinoline; 4-(4-hydroxy-alpha,alpha′-bis(2-methyl-1-pyrrolidinyl)-2,5-xylidinoquinoline; 4-[(4-diethylamino)-1-methylbutyl-amino]-6-methoxyquinoline; 3,4-dihydro-1-(2H)-quinolinecarboxyaldehyde; 1,1′-pentamethylene diquinoleinium diiodide; 8-quinolinol sulfate and amino, aldehyde, carboxylic, hydroxyl, halogen, keto, sulfhydryl and vinyl derivatives or analogs thereof In some instances, an endosomolytic moiety is a small molecule described in Naisbitt et al (1997, J Pharmacol Exp Therapy 280:884-893) and in U.S. Pat. No. 5,736,557.

WSGR DocketNo. 45532-756.30 Cell Penetrating Polypeptide (CPP)

In some aspects, cell penetrating polypeptide comprises positively charged short peptides with 5-30 amino acids. In some aspects, cell penetrating polypeptide comprises arginine or lysine rich amino acid sequences. In some aspects, cell penetrating polypeptide includes any polypeptide or combination thereof listed in Table 9.

TABLE 9

| PEPTIDE | SEQUENCE | SEQ ID NO |
|---|---|---|
| Antennapedia Penetratin (43-58) | RQIKIWFQNRRMKWKK | 358 |
| HIV-1 TAT protein (48-60) | GRKKRRQRRRPPQ | 359 |
| pVEC Cadherin (615-632) | LLIILRRRIRKQAHAHSK | 360 |
| Transportan Galanine/Mastoparan | GWTLNSAGYLLGKINLKALAALAKKIL | 361 |
| MPG HIV-gp41/SV40 T-antigen | GALFLGFLGAAGSTMGAWSQPKKKRKV | 362 |
| Pep-1 HIV-reverse | KETWWETWWTEWSQPKKKRKV | 363 |
| transcriptase/SV40 T-antigen | | |
| Polyarginines | R(n); 6 < n < 12 | 364 |
| MAP | KLALKLALKALKAALKLA | 365 |
| R6W3 | RRWWRRWRR | 366 |
| NLS | CGYGPKKKRKVGG | 367 |
| 8-lysines | KKKKKKKK | 368 |
| ARF (1-22) | MVRRFLVTLRIRRACGPPRVRV | 369 |
| Azurin-p28 | LSTAADMQGVVTDGMASGLDKDYLKPDD | 370 |

### Linkers

In some aspects, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety C. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[P-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, a,a′-p-diaminodiphenyl, diiodo-p-xylene sulfonic acid, N,N′-ethylene-bis(iodoacetamide), or N,N′-hexamethylene-bis(iodoacetamide).

In some aspects, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(7-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such asl-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some instances, the linker comprises a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some aspects, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some aspects, the linker comprises a peptide moiety. In some instances, the peptide moiety comprises at least 2, 3, 4, 5, or 6 more amino acid residues. In some instances, the peptide moiety comprises atmost 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 403), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 404), or Gly-Phe-Leu-Gly (SEQ ID NO: 405). In some instances, the linker comprises a peptide moiety such as: Val-Cit (valine-citrulline), Gly-Gly-Phe-Gly (SEQ ID NO: 403), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 404), or Gly-Phe-Leu-Gly (SEQ ID NO: 405). In some cases, the linker comprises Val-Cit. In some cases, the linker is Val-Cit.

In some aspects, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication NO. WO2015038426.

In some aspects, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some aspects, the linker is a traceless linker or a linker in which after cleavage does not leave behind a linker moiety (e.g., an atom or a linker group) to a binding moiety A, a polynucleotide B, a polymer C, or an endosomolytic moiety D. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, etal., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication NOs. 2014/0127239; 2013/028919; 2014/286970; 2013/0309256; 2015/037360; or 2014/0294851; orPCT Publication NOs. WO2015057699; WO2014080251; WO2014197854; WO2014145090; or WO2014177042.

In some aspects, X₁ and X₂ are each independently a bond or a non-polymeric linker. In some instances, X₁ and X₂ are each independently a bond. In some cases, X₁ and X₂ are each independently a non-polymeric linker.

In some instances, X₁ is a bond or a non-polymeric linker. In some instances, X₁ is a bond. In some instances, X₁ is a non-polymeric linker. In some instances, the linker is a C₁-C₆ alkyl group. In some cases, X₁ is a C₁-C₆ alkyl group, such as for example, a C₅, C₄, C₃, C₂, or C₁ alkyl group. In some cases, the C₁-C₆ alkyl group is an unsubstituted C₁-C₆ alkyl group. As used in the context of a linker, and in particular in the context of X₁, alkyl means a saturated straight or branched hydrocarbon radical containingup to six carbon atoms. In some instances, X₁ includes a homobifunctional linker or a heterobifunctional linker described supra. In some cases, X₁ includes a heterobifunctional linker. In some cases, X₁ includes sMCC. In other instances, X₁ includes a heterobifunctional linker optionally conjugated to a C₁-C₆ alkyl group.

In other instances, X₁ includes sMCC optionally conjugated to a C₁-C₆ alkyl group. In additional instances, X₁ does not include a homobifunctional linker or a heterobifunctional linker described supra.

In some instances, X₂ is a bond or a linker. In some instances, X₂ is a bond. In other cases, X₂ is a linker. In additional cases, X₂ is a non-polymeric linker. In some aspects, X₂ is a C₁-C₆ alkyl group. In some instances, X₂ is a homobifunctional linker or a heterobifunctional linker described supra. In some instances, X₂ is a homobifunctional linker described supra. In some instances, X₂ is a heterobifunctional linker described supra. In some instances, X₂ comprises a maleimide group, such as maleimidocaproyl (mc) or a self-stabilizing maleimide group described above. In some instances, X₂ comprises a peptide moiety, such as Val-Cit. In some instances, X₂ comprises a benzoic acid group, such as PABA. In additional instances, X₂ comprises a combination of a maleimide group, a peptide moiety, and/or a benzoic acid group.

In additional instances, X₂ comprises a me group. In additional instances, X₂ comprises a mc-val-cit group. In additional instances, X₂ comprises a val-cit-PABA group. In additional instances, X₂ comprises a mc-val-cit-PABA group.

### Methods of Use

Muscle atrophy refers to a loss of muscle mass and/or to a progressive weakening and degeneration of muscles. In some cases, the loss of muscle mass and/or the progressive weakening and degeneration ofmuscles occurs due to a high rate of protein degradation, a low rate of protein synthesis, or a combination of both. In some cases, a high rate of muscle protein degradation is due to muscle protein catabolism (i.e., the breakdown of muscle protein in order to use amino acids as substrates for gluconeogenesis).

In one embodiment, muscle atrophy refers to a significant loss in muscle strength. By significant loss in muscle strength is meant a reduction of strength in diseased, injured, or unused muscle tissue in a subject relative to the same muscle tissue in a control subject. In an embodiment, a significant loss in muscle strength is a reduction in strength of at least 10%, at least 15%, at least 20%, at least 25%, at least 30%, at least 35%, at least 40%, at least 45%, at least 50%, or more relative to the same muscle tissue in a control subject. In another embodiment, by significant loss in muscle strength is meant a reduction of strength in unused muscle tissue relative to the muscle strength of the same muscle tissue in the same subject prior to a period of nonuse. In an embodiment, a significant loss in muscle strength is a reduction of at least 10%, at least 15%, at least 20%, at least 25%, at least 30%, at least 35%, at least 40%, at least 45%, at least 50%, or more relative to the muscle strength of the same muscle tissue in the same subject prior to a period of nonuse.

In another embodiment, muscle atrophy refers to a significant loss in muscle mass. By significant loss in muscle mass is meant a reduction of muscle volume in diseased, injured, or unused muscle tissue in a subject relative to the same muscle tissue in a control subject. In an embodiment, a significant loss of muscle volume is at least 10%, at least 15%, at least 20%, at least 25%, at least 30%, at least 35%, at least 40%, at least 45%, at least 50%, or more relative to the same muscle tissue in a control subject. In another embodiment, by significant loss in muscle mass is meant a reduction of muscle volume in unused muscle tissue relative to the muscle volume of the same muscle tissue in the same subject prior to a period of nonuse. In an embodiment, a significant loss in muscle tissue is at least 10%, at least 15%, at least 20%, at least25%, at least 30%, at least 35%, at least 40%, at least 45%, at least 50%, ormore relative to the muscle volume of the same muscle tissue in the same subject prior to a period of nonuse.

Muscle volume is optionally measured by evaluating the cross-section area of a muscle such as by Magnetic Resonance Imaging (e.g., by a muscle volume/cross-section area (CSA) MRI method).

In some aspects, described herein is a method of treating muscle atrophy in a subject, which comprises providing polynucleic acid molecule described herein and administering to the subject a therapeutically effective amount of a polynucleic acid molecule described herein or a polynucleic acid molecule conjugate described herein to reduces a quantity of the mRNA transcript of human DUX4. In some embodiments, the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 412-420 or 430-438. In some embodiments, the polynucleic acid molecule comprises a sense strand and/or an antisense strand, and the antisense is identical to a sequence selected from SEQ ID NOs: 412-420 or 430-438. In some embodiments, the polynucleic acid molecule comprises a sense strand and/or an antisense strand, and the sense strand comprises at least 80%, at least 85%, at least 90%, at least 95%, at least 96%, at least 97%, at least 98%, at least 99% identical to a sequence selected from SEQ ID NOs: 142, 146, 196, or 201-206. In some embodiments, the polynucleic acid molecule comprises a sense strand and/or an antisense strand, and the sense strand is identical to a sequence selected from SEQ ID NOs: 142, 146, 196, or 201-206.

In some instances, the muscle atrophy is associated with Facioscapulohumeral muscular dystrophy (FSHD). The polynucleic acid moiety mediates RNA interference against the human DUX4 as to modulating muscle atrophy in a subject. In some aspects, expression of one or more marker genes that are affected by DUX4 expression is also altered or modulated (e.g., decreased) by the decreased expression of human DUX4. The marker genes includes, but not limited to, MBD3L2, TRIM43, PRAMEF1, ZSCAN4, KHDC1L, LEUTX, WFDC3, ILVBL, SLC15A2, and SORD. In some aspects, the expression of one or more marker genes is decreased at least 10%, 15%, 20%, 25%, 30%, 35%, 40%, 45%, or 50% compared to untreated cells. In some aspects, the expression of one or more marker genes, as a group or a composite, is decreased at least 10%, 15%, 20%, 25%, 30%, 35%, 40%, 45%, or 50% compared to untreated cells.

In some aspects, described herein is a method of treating muscle atrophy in a subject, which comprises providing an siRNA-antibody conjugate described herein and administering to the subject a therapeutically effective amount of the siRNA-antibody conjugate described herein and reducing the levels of mRNA transcript of human DUX4 in said subject. In some instances, the muscle atrophy is associated with FSHD. The siRNA-antibody conjugate mediates RNA interference against the human DUX4 mRNA as to treat muscle atrophy in the subject, which comprises administering to the subject a therapeutically effective amount of the siRNA-antibody conjugate described herein and reducing the levels of mRNA transcript of human DUX4 in said subject.

In some aspects, described herein is a method of treating muscle atrophy in a subject, which comprises providing a DUX4 siRNA-antibody conjugate (DUX4 siRNA-conjugate or DUX4-AOC) described herein and administering to the subject a therapeutically effective amount of the DUX4 siRNA-antibody conjugate described herein and reducing the levels of mRNA transcript of human DUX4 in said subject. In some instances, the muscle atrophy is associated with FSHD. The DUX4 siRNA-antibody conjugate mediates RNA interference against the human DUX4 mRNA as to treat muscle atrophy in the subject, which comprises administering to the subject a therapeutically effective amount of the DUX4 siRNA-antibody conjugate described herein and reducing the levels of mRNA transcript of human DUX4 in said subject.

In some aspects, described herein is a method of treating FSHD in a subject, which comprises providing a DUX4 siRNA-antibody conjugate (DUX4 siRNA conjugate or DUX4-AOC) described herein and administering to the subject a therapeutically effective amount of the DUX4 siRNA-antibody conjugate described herein and reducing the levels of mRNA transcript of human DUX4 in said subject. In some instances, the FSHD is FSHD type 1 (FSHD1). In some instances, the FSHD is FSHD type 2 (FSHD2). The DUX4 siRNA-antibody conjugate mediates RNA interference against the human DUX4 mRNA as to treat FSHD in the subject, which comprises administering to the subject a therapeutically effective amount of the DUX4 siRNA-conjugate described herein and reducing the levels of mRNA transcript of human DUX4 in said subject. In some aspects, expression levels of one or more marker genes that are affected by DUX4 expression are also altered or modulated by the decreased expression levels of human DUX4. The DUX4 biomarker genes include but are not limited to MBD3L2, TRIM43, PRAMEF1, ZSCAN4, KHDC1L, LEUTX, WFDC3, ILVBL, SLC15A2, and SORD.

In some aspects, described herein is a method of alleviating symptoms in a subject with FSHD, which comprises providing a DUX4 siRNA-antibody conjugate (DUX4-siRNA conjugate or DUX4-AOC) described herein and administering to the subject a therapeutically effective amount of the siRNA conjugate described herein by reducing the levels of mRNA transcript of human DUX4. In some instances, the FSHD is FSHD type 1 (FSHD1). In some instances, the FSHD is FSHD type 2 (FSHD2). In another aspects, described herein is a method of alleviating symptoms in a FSHD patient, which comprises providing an siRNA conjugate described herein and administering to the FSHD patient a therapeutically effective amount of the siRNA conjugate describes herein by reducing the levels of mRNA transcript of human DUX4 or reducing the levels of DUX4 protein.

In some instances, the symptoms ofFSHD affect skeletal muscles. The skeletal muscles affected by FSHD include muscles around the eyes and mouth, muscle of the shoulders, muscle of the upper arms, muscle of the lower legs, abdominal muscles and hip muscles. In some instances, the symptoms of FSHD also affects vision and hearing. In some instances, the symptoms of FSHD also affect the function of the heart or lungs. In some instances, the symptoms of FSHD include muscle weakness, muscle atrophy, muscle dystrophy, pain inflammation, contractures, scoliosis, lordosis, hypoventilation, abnormalities of the retina, exposure to keratitis, mild hearing loss, and EMG abnormality.

In some aspects, described herein is a method of improving skeletal muscle functions in a FSHD patient comprising the step of administering to the FSHD patient a therapeutically effective amount of the siRNA conjugate described herein by reducing the levels of mRNA transcript of human DUX4 or reducing the levels of DUX4 protein. In some instances, FSHD is FSHD type 1 (FSHD1). In some instances, FSHD is FSHD type 2. In some aspects, described herein is a method of improving skeletal muscle functions, vision, hearing, heart functions or lung functions in a patient suffering from FSHD comprising the step of administering to the FSHD patient a therapeutically effective amount of the siRNA conjugate described herein by reducing the levels of mRNA transcript of human DUX4 or reducing the levels of DUX4 protein.

In some aspects, described herein is a method of treating FSHD in a subject, which comprises providing an antisense oligonucleotide (ASO) antibody conjugate (ASO conjugate) described herein and administering to the subject a therapeutically effective amount of the ASO-antibody conjugate described herein and reducing the levels of mRNA transcript of human DUX4 in said subject. In some instances, FSHD is FSHD type 1 (FSHD1). In some instances, FSHD is FSHD type 2. The ASO-antibody conjugate mediates RNA interference against the human DUX4 mRNA as to treat FSHD in the subject, which comprises administering to the subject a therapeutically effective amount of the ASO-antibody conjugate described herein and reducing the levels of mRNA transcript of human DUX4 in said subject. In some aspects, expression levels of one or more marker genes that are affected by DUX4 expression is also altered or modulated by the decreased expression levels of human DUX4. The DUX4 biomarker genes include but are not limited to MBD3L2, TRIM43, PRAMEF1, ZSCAN4, KHDC1L, LEUTX, WFDC3, ILVBL, SLC15A2, and SORD.

In some aspects, described herein is a method of treating FSHD in a subject. In some instances, the FSHD subject suffers from FSHD1. In other instances, the FSHD subject suffers from FSHD2. In another embodiment, the FSHD subject has muscle cells abnormally expressing DUX4 protein caused by the genetic and epigenetic molecular changes in the D4Z4 region of the long arm of chromosome 4. The genetic molecular changes in the muscle cells are mutations leading to the contraction of the D4Z4 region containing 1-10repeats instead of the normal 11 to 100 repeats of chromosome 4 of the FSHD subject. The epigenetic molecular changes in the muscle cells are changes leading to the hypomethylation of the D4Z4 region of chromosome 4 of the FSHD subject. In some instances, the muscle cells are skeletal muscle cells.

### Pharmaceutical Formulation

In some aspects, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, orintracranial) administration.

In other instances, the pharmaceutical composition describe herein is formulated for oral administration. In still other instances, the pharmaceutical composition describe herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, a-chymotrypsin, polylysine, polyarginine, histone, protamine, ovalbumin or dextrin or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some aspects, the pharmaceutical formulation comprises a delivery vector, e.g., a recombinant vector, the delivery ofthe polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

In some aspects, the pharmaceutical formulation includes a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice ofPharmacy, Nineteenth Ed (Easton, Pa.: Mack Publishing Company, 1995); Hoover, John E., Remington's PharmaceuticalSciences, Mack Publishing Co., Easton, Pennsylvania 1975; Liberman, H. A. and Lachman, L., Eds., PharmaceuticalDosage Forms, Marcel Decker, New York, N.Y., 1980; and PharmaceuticalDosage Forms and Drug Delivery Systems, Seventh Ed. (Lippincott Williams & Wilkinsl999).

In some instances, the pharmaceutical formulation further includes pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodiumbicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulation further includes diluent which are used to stabilize compounds because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulation includes disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol©), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulation includes filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O_Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some aspects, the pharmaceutical compositions described herein are administered for therapeutic applications. In some aspects, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, once in two months, once in three months, once in fourmonths, once in five months, once in six months or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some aspects, one or more pharmaceutical compositions are administered simultaneously, sequentially, or at an interval period of time. In some aspects, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some aspects, two or more different pharmaceutical compositions are co-administered.

In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55% 60%, 65%, 70%, 75%, 80%, 85%, 90%, 95%, or 100%.

Once improvement of the patient's conditions has occurred, a maintenance dose is administered if necessary. Subsequently, the dosage orthe frequency of administration, or both, can be reduced, as a function of the symptoms, to a level at which the improved disease, disorder or condition is retained.

In some aspects, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compoundused, the disease or condition to be treated, the mode of administration, the requirements of the individual subject, the severity of the disease or condition being treated, and the judgment of the practitioner.

In some aspects, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain aspects, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain aspects, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some aspects, the mammal is a human. In some aspects, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The term “therapeutically effective amount” relates to an amount of a polynucleic acid molecule conjugate that is sufficient to provide a desired therapeutic effect in a mammalian subject. In some cases, the amount is single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. Naturally, dosage levels of the particular polynucleic acid molecule conjugate employed to provide a therapeutically effective amount vary in dependence of the type of injury, the age, the weight, the gender, the medical condition of the subject, the severity of the condition, the route of administration, and the particular inhibitor employed. In some instances, therapeutically effective amounts of polynucleic acid molecule conjugate, as described herein, is estimated initially from cell culture and animal models. For example, IC₅₀values determined in cell culture methods optionally serve as a starting point in animal models, while IC₅₀ values determined in animal models are optionally used to find a therapeutically effective dose in humans.

Skeletal muscle, or voluntary muscle, is generally anchored by tendons to bone and is generally used to effect skeletal movement such as locomotion or in maintaining posture.

Although some control of skeletal muscle is generally maintained as an unconscious reflex (e.g., postural muscles or the diaphragm), skeletal muscles react to conscious control. Smooth muscle, or involuntary muscle, is found within the walls of organs and structures such as the esophagus, stomach, intestines, uterus, urethra, and blood vessels.

Skeletal muscle is further divided into two broad types: Type I (or “slow twitch”) and Type II (or “fast twitch”). Type I muscle fibers are dense with capillaries and are rich in mitochondria and myoglobin, which gives Type I muscle tissue a characteristic red color. In some cases, Type I muscle fibers carries more oxygen and sustain aerobic activity using fats or carbohydrates for fuel. Type I muscle fibers contract for long periods of time but with little force. Type IImuscle fibers are further subdivided into three major subtypes (IIa, IIx, and IIb) that vary in both contractile speed and force generated. Type II muscle fibers contract quickly and powerfully but fatigue very rapidly, and therefore produce only short, anaerobic bursts of activity before muscle contraction becomes painful.

Unlike skeletal muscle, smooth muscle is not under conscious control.

Cardiac muscle is also an involuntary muscle but more closely resembles skeletal muscle in structure and is found only in the heart. Cardiac and skeletal muscles are striated in that they contain sarcomeres that are packed into highly regular arrangements of bundles. By contrast, the myofibrils of smooth muscle cells are not arranged in sarcomeres and therefore are not striated.

Muscle cells encompass any cells that contribute to muscle tissue. Exemplary muscle cells include myoblasts, satellite cells, myotubes, and myofibril tissues.

As used here, muscle force is proportional to the cross-sectional area (CSA), and muscle velocity is proportional to muscle fiber length. Thus, comparing the cross-sectional areas and muscle fibers between various kinds of muscles is capable of providing an indication of muscle atrophy. Various methods are known in the art to measure muscle strength and muscle weight, see, for example, “Musculoskeletal assessment: Joint range of motion and manual muscle strength” by Hazel M. Clarkson, published by Lippincott Williams & Wilkins, 2000. The production of tomographic images from selected muscle tissues by computed axial tomography and sonographic evaluation are additional methods of measuring muscle mass.

The term antibody oligonucleotide conjugate (AOC) refers to an antibody conjugated to a nucleotide.

The term “siRNA conjugate” or “siRNA-antibody conjugate” refers to an antibody conjugated to an siRNA.

The term “DUX4 siRNA-conjugate” or “DUX4 siRNA-antibody conjugate” refers to an antibody conjugated to an siRNA hybridizing to a target sequence of the human DUX4 mRNA.

The term “DUX4-AOC” refers to an antibody conjugated to an siRNA hybridizing to a target sequence of the human DUX4 mRNA.

### Embodiments

Embodiment 1. A polynucleic acid molecule conjugate comprising: an antibody or antigen binding fragment thereof conjugated to a polynucleic acid molecule that hybridizes to a target sequence ofDUX4; and wherein the polynucleic acid molecule conjugate mediates RNA interference against the DUX4.

Embodiment 2. The polynucleic acid molecule conjugate of Embodiment 1, wherein the antibody or antigen binding fragment thereof comprises a non-human antibody or antigen binding fragment thereof, a human antibody or antigen binding fragment thereof, a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof.

Embodiment 3. The polynucleic acid molecule conjugate of Embodiment 1 or 2, wherein the antibody or antigen binding fragment thereof is an anti-transferrin receptor antibody or antigen binding fragment thereof.

Embodiment 4. The polynucleic acid molecule conjugate of any one of Embodiments 1-3, wherein the polynucleic acid molecule comprises a sense strand and/or an antisense strand, and wherein the sense strand and/or the antisense strand each independently comprises at least one 2′ modified nucleotide, at least one modified internucleotide linkage, or at least one inverted abasic moiety.

Embodiment 5. The polynucleic acid molecule conjugate of any one of Embodiments 1-4, wherein the polynucleotide hybridizes to at least 8 contiguous bases of the target sequence of DUX4.

Embodiment 6. The polynucleic acid molecule conjugate of any one of Embodiments 1-5, wherein the polynucleotide is from about 8 to about 50 nucleotides in length or from about 10 to about 30 nucleotides in length.

Embodiment 7. The polynucleic acid molecule conjugate of any one of Embodiments 1-6, wherein the polynucleic acid molecule comprises a sense strand and/or an antisense strand, and the sense strand comprises at least 80%, at least 85%, at least 90%, at least 95%, at least 96%, at least 97%, at least 98%, at least 99% identical to a sequence selected from SEQ ID NOs: 1-70 or SEQ ID NOs: 141-210.

Embodiment 8. The polynucleic acid molecule conjugate of any one of Embodiments 1-7, wherein the polynucleic acid molecule comprises a sense strand and/or an antisense strand, and the sense strand comprises at least 80%, at least 85%, at least 90%, at least 95%, at least 96%, at least 97%, at least 98%, at least 99% identical to a sequence selected from SEQ ID NOs: 142, 146, 196, 201-206.

Embodiment 9. The polynucleic acid molecule conjugate of any one of Embodiments 1-8, wherein the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 71-140 or SEQ ID NOs: 211-280.

Embodiment 10. The polynucleic acid molecule conjugate of any one of Embodiments 1-9, wherein the polynucleic acid molecule comprises a sense strand and/or an antisense strand, and the antisense strand comprises at least 80%, at least 85%, at least 90%, at least 95%, at least 96%, at least 97%, at least 98%, at least 99% identical to a sequence selected from SEQ ID NOs: 412-420 and 430-438.

Embodiment 11. The polynucleic acid molecule conjugate of any one of Embodiments 1-10, wherein the polynucleotide comprises at least one 2′ modified nucleotide, and further wherein the 2′ modified nucleotide: comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA) modified nucleotide; comprises locked nucleic acid (LNA) or ethylene nucleic acid (ENA); or comprises a combination thereof.

Embodiment 12. The polynucleic acid molecule conjugate of any one of Embodiments 1-11, wherein the at least one modified internucleotide linkage comprises a phosphorothioate linkage or a phosphorodithioate linkage.

Embodiment 13. The polynucleic acid molecule conjugate of any one of Embodiments 1-12, wherein the polynucleic acid molecule comprises 3 or more 2′ modified nucleotides selected from 2′-O-methyl and 2′-deoxy-2′-fluoro.

Embodiment 14. The polynucleic acid molecule conjugate of any one of Embodiments 1-13, wherein the polynucleic acid molecule comprises a 5′-terminal vinylphosphonate modified nucleotide.

Embodiment 15. The polynucleic acid molecule conjugate of any one of Embodiments 1-14, wherein the 5′-terminal vinylphosphonate modified nucleotide is selected from:

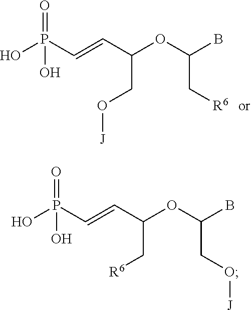

wherein B is a heterocyclic base moiety; R6 is selected from hydrogen, halogen, alkyl or alkoxy; and J is an internucleotide linking group linking to the adjacent nucleotide of the polynucleotide.

Embodiment 16. The polynucleic acid molecule conjugate of any one of Embodiments 1-15, wherein the 2′ modified nucleotide is 2′-O-methyl modified nucleotide, and 2′-O-methyl modified nucleotide is at the 5′-end of the sense strand and/or the antisense strand.

Embodiment 17. The polynucleic acid molecule conjugate of Embodiment 16, wherein the 2′-O-methyl modified nucleotide is a purine nucleotide.

Embodiment 18. The polynucleic acid molecule conjugate of Embodiment 16, wherein the 2′-O-methyl modified nucleotide is a pyridine nucleotide.

Embodiment 19. The polynucleic acid molecule conjugate of any one of Embodiments 16-18, wherein the sense and/or antisense strands comprise at least two, three, four consecutive the 2′-O-methyl modified nucleotides at the 5′-end.

Embodiment 20. The polynucleic acid molecule conjugate of any one of Embodiments 1-19, wherein the polynucleic acid molecule conjugate comprises a linker connecting the antibody or antigen binding fragment thereof to the polynucleic acid molecule.

Embodiment 21. The polynucleic acid molecule conjugate of Embodiment 20, wherein the linker is C1-C6 alkyl linker.

Embodiment 22. The polynucleic acid molecule conjugate of Embodiment 20, wherein the linker is a homobifunctional linker or heterobifunctional linker, and comprises a maleimide group, a dipeptide moiety, a benzoic acid group, or its derivative thereof.

Embodiment 23. The polynucleic acid molecule conjugate of Embodiment 20, wherein the linker is a cleavable or non-cleavable linker.

Embodiment 24. The polynucleic acid molecule conjugate of any one of Embodiments 1-23, wherein a ratio between the polynucleic acid molecule and the antibody or antigen binding fragment thereof is about 1:1, 2:1, 3:1, or 4:1.

Embodiment 25. The polynucleic acid molecule conjugate of any one of Embodiments 1-24, wherein the polynucleic acid molecule mediates RNA interference against the human DUX4 and modulates muscle atrophy in a subject.

Embodiment 26. The polynucleic acid molecule conjugate of Embodiment 25, wherein the RNA interference comprises reducing expression of the mRNA transcript of DUX4 gene at least 50%, at least 60%, or at least 70% or more compared to a quantity of the mRNA transcript of DUX4 gene in an untreated cell.

Embodiment 27. The polynucleic acid molecule conjugate of any one of Embodiments 25-26, wherein the RNA interference comprises affecting expression of a marker gene selected from a group consisting of MBD3L2, TRIM43, PRAMEF1, ZSCAN4, KHDC1L, and LEUTX in a cell.

Embodiment 28. The polynucleic acid molecule conjugate of any one of Embodiments 25-26, wherein the RNA interference comprises affecting expression of a marker gene selected from a group consisting of WFDC3, ILVBL, SLC15A2, and SORD in a cell

Embodiment 29. The polynucleic acid molecule conjugate of Embodiment 28, wherein the affecting expression of the marker gene is reducing expression of the marker gene at least 20%, at least 30%, at least 40%, at least 50%, at least 60% or more.

Embodiment 30. The polynucleic acid molecule conjugate of any one of Embodiments 25-29, wherein the muscle dystrophy is Facioscapulohumeral muscular dystrophy (FSHD).

Embodiment 31. The polynucleic acid molecule conjugate of any one of Embodiments 1-30, wherein the polynucleic acid molecule conjugate comprises a molecule of Formula (I):

A-X-B

### (Formula I)

wherein,
A is the antibody or antigen binding fragment thereof;
B is the polynucleic acid molecule that hybridizes to a target sequence of DUX4;
X is a bond or a non-polymeric linker; and
wherein X is conjugated to a cysteine residue of A.

Embodiment 32. A pharmaceutical composition comprising:
a polynucleic acid molecule conjugate of Embodiments 1-31; and a pharmaceutically acceptable excipient.

Embodiment 33. The pharmaceutical composition of Embodiment 32, wherein the pharmaceutical composition is formulated as a nanoparticle formulation.

Embodiment 34. The pharmaceutical composition of any one of Embodiments 32-33, wherein the pharmaceutical composition is formulated for parenteral, oral, intranasal, buccal, rectal, transdermal, intravenous, subcutaneous, or intrathecal administration.

Embodiment 35. A method for treating muscular dystrophy in a subject in need thereof, comprising:
providing a polynucleic acid conjugate of any one of Embodiments 1-34; and administering the polynucleic acid conjugate to the subject in need thereof to treat the muscular dystrophy, wherein the polynucleic acid conjugate reduces a quantity of the mRNA transcript of human DUX4.

Embodiment 36. The method of Embodiment 35, wherein the polynucleic acid moiety mediates RNA interference against the human DUX4 and modulates muscle atrophy in a subject.

Embodiment 37. The method of Embodiment 36, wherein the RNA interference comprises affecting expression of a marker gene selected from a group consisting of MBD3L2, TRIM43, PRAMEF1, ZSCAN4, KHDC1L, LEUTX, WFDC3, ILVBL, SLC15A2, and SORD in a cell affected by a muscle dystrophy.

Embodiment 38. The method of any one of Embodiments 35-38, wherein the muscular dystrophy is Facioscapulohumeral muscular dystrophy (FSHD).

Embodiment 39. Use of the polynucleic acid molecule conjugate of any one of Embodiments 1-30 or the pharmaceutical composition of any one of Embodiments 32-34 for treating in a subject diagnosed with or suspected to have Facioscapulohumeral muscular dystrophy (FSHD).

Embodiment 40. Use of the polynucleic acid molecule conjugate of any one of Embodiments 1-30 or the pharmaceutical composition of any one of Embodiments 32-34 for manufacturing a medicament for treating in a subject diagnosed with or suspected to have Facioscapulohumeral muscular dystrophy (FSHD).

Embodiment 41. A kit comprising a polynucleic acid molecule conjugate of Embodiments 1-31 or the pharmaceutical composition of any one of Embodiments 32-34.

### EXAMPLES

These examples are provided for illustrative purposes only and not to limit the scope of the claims provided herein.

### Example 1. Bioinformatic siRNA Library Design Against Human Full Length DUX4 Transcript

FIG. 2 shows a flowchart ofin silico selection process of DUX4 siRNA. Sequences of all siRNAs that can binds to DUX4, or a pre-determined region of the DUX4 are collected to generate a starting set of DUX4 siRNA. From the starting set of DUX siRNAs, the first eliminating step comprises eliminating one or more DUX siRNAs that has single nucleotide polymorphism (SNP) and/or MEF<−5. Then, the second eliminating step comprises eliminating DUX siRNAs with 0 and 1 MM in the human transcriptome (such that only hits allowed are DUX, DUX5, and DBET). Then, the third eliminating step comprises eliminating DUX siRNAs with 0 mismatch (MM) in the human intragenic regions (such that only hits allowed are DUX1, DUX5 and DBET pseudogenes). Then, the next eliminating step comprises eliminating DUX siRNAs with a MM to DUX4 human sequence used in FLExDUX4 FSHD mouse model. Then, the next step is carrying forward only or one or more DUX siRNAs with predicted viability>60.

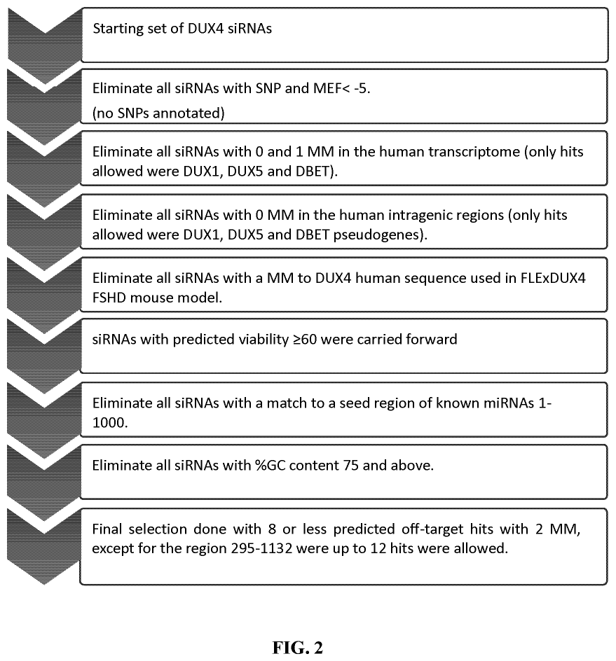

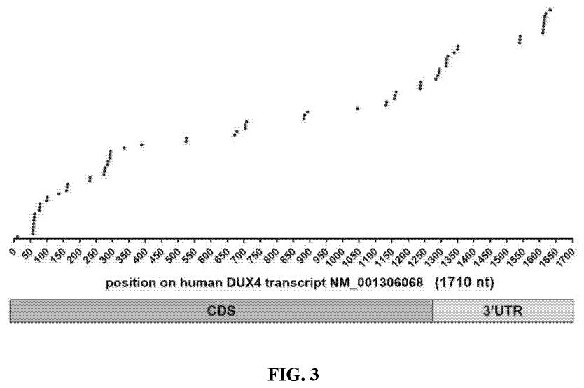

Next, the eliminating step comprises eliminating one or more DUX siRNAs with a match to a seed region of known miRNAs 1-1000. Then, the eliminating step continues with eliminating DUX siRNAs molecule with % GC content 75 and above. Then, the final selection process comprises with eight or fewer predicted off-target hits with 2 MM, except for the region 295-1132, forwhichup to 12 hits are allowed. Using such series of selection steps, final 70 candidate DUX siRNAs could be selected from a starting set of 1694 DUX siRNAs. FIG. 3 shows the location and numbers of such selected DUX4 siRNA in the DUX4 mRNA transcript (NM_001306068).

Identified siRNA candidates share common characteristics in their sequences as shown below in Table 10 The identified siRNAs have mostly 2′-O-Me modifications, with 2′-F modifications only located on sense strand at positions 7, 8, 9 for all 3 DUX4 templates. The 2′-O-Me modifications, with 2′-F modifications, are located on antisense strand at positions 1, 2, 6, 14, 16 for the DUX4 template 1 and at positions 2, 6, 14, 16 for the DUX4 templates 1 and 2.

Also, the identified siRNAs comprises 4 phosphorothioate modifications on each strand, located at the final 2 linkages of each 5′ and 3′ terminus. The identified siRNAs further comprises “Uf” at the first position of 5′ end of the antisense strand for the DUX4 template 1, regardless of the actual target mRNA sequence (coupled with “a” at the last position at the 3′ end of the sense strand) and comprises “vpN” at the first position of the 5′ end of the antisense strand for the DUX4 template 3. The identified siRNAs further comprises “uu” overhang at the 3′ end of the antisense strand only, with no overhang at the 3′ end of the sense strand. The optimization of the identified siRNAs may comprise a vinyl phosphonate nucleotide, an inverted abasic moiety, or an amine linker to the passenger strand or the guide strand.

TABLE 10

| | Sense Strand Sequence | Antisense Strand Sequence |
|---|---|---|
| Duplex Name | (5′-3′) (passenger strand) | (5′-3′) (guide strand) |
| DUX4 template 1 | nsnsnnnnNfNfNfnnnnnnnsnsa | UfsNfsnnnNfnnnnnnnNfnNfnnsusu |
| DUX4 template 2 | nsnsnnnnNfNfNfnnnnnnnsnsa | usNfsnnnNfnnnnnnnNfnNfnnnsusu |
| DUX4 template 3 | nsnsnnnnNfNfNfnnnnnnnsnsa | vpNsNfsnnnNfnnnnnnnNfnNfnnnsusu |

vpN = vinyl phosphonate VpUq; upper case (N) = 2′-OH (ribo); lower case (n) = 2′-O-Me(methyl) dN = 2′-H (deoxy); Nf = 2′F (fluoro); s = phosphorothioate backbone modification; iB = inverted abasic

Tables 11, 12, 13, 14, and 15 illustrate identified siRNA candidates for the regulation of human DUX4.

TABLE 11

| | 19mer | SEQ | | SEQ | |
|---|---|---|---|---|---|
| | start | ID | sense/passenger_seq | ID | antisense/guide_seq |
| Name | site | NO | (5′-3′) | NO | (5′-3′) |
| NM_001306068_11_29 | 11 | 1 | cgacaccctcggacagcac | 71 | gtgctgtccgagggtgtcg |
| NM_001306068_57_75 | 57 | 2 | acggcgacggagactcgtt | 72 | aacgagtctccgtcgccgt |
| NM_001306068_58_76 | 58 | 3 | cggcgacggagactcgttt | 73 | aaacgagtctccgtcgccg |
| NM_001306068_59_77 | 59 | 4 | ggcgacggagactcgtttg | 74 | caaacgagtctccgtcgcc |
| NM_001306068_60_78 | 60 | 5 | gcgacggagactcgtttgg | 75 | ccaaacgagtctccgtcgc |
| NM_001306068_61_79 | 61 | 6 | cgacggagactcgtttgga | 76 | tccaaacgagtctccgtcg |
| NM_001306068_62_80 | 62 | 7 | gacggaaactcgtttggac | 77 | gtccaaacgagtctccgtc |
| NM_001306068_63_81 | 63 | 8 | acggagactcgtttggacc | 78 | ggtccaaacgagtctccgt |
| NM_001306068_77_95 | 77 | 9 | ggaccccgagccaaagcga | 79 | tcgctttggctcggggtcc |
| NM_001306068_78_96 | 78 | 10 | gaccccgagccaaagcgag | 80 | ctcgctttggctcggggtc |
| NM_001306068_79_97 | 79 | 11 | accccgagccaaagcgagg | 81 | cctcgctttggctcggggt |
| NM_001306068_99_117 | 99 | 12 | cctgcgagcctgctttgag | 82 | ctcaaagcaggctcgcagg |
| NM_001306068_102_120 | 102 | 13 | gcgagcctgctttgagcgg | 83 | ccgctcaaagcaggctcgc |
| NM_001306068_137_155 | 137 | 14 | tcgccaccagagaacggct | 84 | agccgttctctggtggcga |
| NM_001306068_160_178 | 160 | 15 | caggccatcggcattccgg | 85 | ccggaatgccgatggcctg |
| NM_001306068_162_180 | 162 | 16 | ggccatcggcattccggag | 86 | ctccggaatgccgatggcc |
| NM_001306068_163_181 | 163 | 17 | gccatcggcattccggagc | 87 | gctccggaatgccgatggc |
| NM_001306068_231_249 | 231 | 18 | gcaccggcgggaatctcgg | 88 | ccgagattcccgccggtgc |
| NM_001306068_232_250 | 232 | 19 | caccggcgggaatctcggc | 89 | gccgagattcccgccggtg |
| NM_001306068_274_292 | 274 | 20 | ccagaaggccggcgaaagc | 90 | gctttcgccggccttctgg |
| NM_001306068_276_294 | 276 | 21 | agaaggccggcgaaagcgg | 91 | ccgctttcgccggccttct |
| NM_001306068_277_295 | 277 | 22 | gaaggccggcgaaagcgga | 92 | tccgctttcgccggccttc |
| NM_001306068_285_303 | 285 | 23 | gcgaaagcggaccgccgtc | 93 | gacggcggtccgctttcgc |
| NM_001306068_287_305 | 287 | 24 | gaaagcggaccgccgtcac | 94 | gtgacggcggtccgctttc |
| NM_001306068_292_310 | 292 | 25 | cggaccgccgtcaccggat | 95 | atccggtgacggcggtccg |
| NM_001306068_293_311 | 293 | 26 | ggaccgccgtcaccggatc | 96 | gatccggtgacggcggtcc |
| NM_001306068_294_312 | 294 | 27 | gaccgccgtcaccggatcc | 97 | ggatccggtgacggcggtc |
| NM_001306068_389_407 | 389 | 28 | agacgggcctcccggagtc | 98 | gactccgggaggcccgtct |
| NM_001306068_524_542 | 524 | 29 | cctcgtgggtcgccttcgc | 99 | gcgaaggcgacccacgagg |
| NM_001306068_525_543 | 525 | 30 | ctcgtgggtcgccttcgcc | 100 | ggcgaaggcgacccacgag |
| NM_001306068_679_697 | 679 | 31 | gaggggatctcccaacctg | 101 | caggttgggagatcccctc |
| NM_001306068_704_722 | 704 | 32 | cgcgcggggatttcgccla | 102 | taggcgaaatccccgcgcg |
| NM_001306068_705_723 | 705 | 33 | gcgcggggatttcgcctac | 103 | gtaggcgaaatccccgcgc |
| NM_001306068_708_726 | 708 | 34 | cggggatttcgcctacgcc | 104 | ggcglaggcgaaatccccg |
| NM_001306068_893_911 | 893 | 35 | tgcttgcgccacccacgtc | 105 | gacgtgggtggcgcaagca |
| NM_001306068_1132_1150 | 1132 | 36 | ctggcgagcccggagtttc | 106 | gaaactccgggctcgccag |
| NM_001306068_1134_1152 | 1134 | 37 | ggcgagcccggagtttctg | 107 | cagaaactccgggctcgcc |
| NM_001306068_1158_1176 | 1158 | 38 | ggcgcaacctctcctagaa | 108 | ttctaggagaggttgcgcc |
| NM_001306068_1159_1177 | 1159 | 39 | gcgcaacctctcctagaaa | 109 | tttctaggagaggttgcgc |
| NM_001306068_1163_1181 | 1163 | 40 | aacctctcctagaaacgga | 110 | tccgtttctaggagaggtt |
| NM_001306068_1236_1254 | 1236 | 41 | cagcgaggaagaataccgg | 111 | ccggtattcttcctcgctg |
| NM_001306068_1237_1255 | 1237 | 42 | agcgaggaagaataccggg | 112 | cccggtattcttcctcgct |
| NM_001306068_1238_1256 | 1238 | 43 | gcgaggaagaataccgggc | 113 | gcccggtattcttcctcgc |
| NM_001306068_1284_1302 | 1284 | 44 | gttgggacggggtcgggtg | 114 | cacccgaccccgtcccaac |
| NM_001306068_1290_1308 | 1290 | 45 | acggggtcgggtggttcgg | 115 | ccgaaccacccgaccccgt |
| NM_001306068_1294_1312 | 1294 | 46 | ggtcgggtggttcggggca | 116 | tgccccgaaccacccgacc |
| NM_001306068_1295_1313 | 1295 | 47 | gtcgggtggttcggggcag | 117 | ctgccccgaaccacccgac |
| NM_001306068_1315_1333 | 1315 | 48 | gcggtggcctctctttcgc | 118 | gcgaaagagaggccaccgc |
| NM_001306068_1316_1334 | 1316 | 49 | cggtggcctctctttcgcg | 119 | cgcgaaagagaggccaccg |
| NM_001306068_1317_1335 | 1317 | 50 | ggtggcctctctttcgcgg | 120 | ccgcgaaagagaggccacc |
| NM_001306068_1321_1339 | 1321 | 51 | gcctctctttcgcggggaa | 121 | ttccccgcgaaagagaggc |
| NM_001306068_1340_1358 | 1340 | 52 | cacctggctggctacggag | 122 | ctccgtagccagccaggtg |
| NM_001306068_1350_1368 | 1350 | 53 | gctacggaggggcgtgtct | 123 | agacacgcccctccgtagc |
| NM_001306068_1351_1369 | 1351 | 54 | ctacggaggggcgtgtctc | 124 | gagacacgcccctccgtag |
| NM_001306068_1539_1557 | 1539 | 55 | acgtgcaagggagctcgct | 125 | agcgagctcccttgcacgt |
| NM_001306068_1540_1558 | 1540 | 56 | cgtgcaagggagctcgctg | 126 | cagcgagctcccttgcacg |
| NM_001306068_1541_1559 | 1541 | 57 | gtgcaagggagctcgctgg | 127 | ccagcgagctcccttgcac |
| NM_001306068_1610_1628 | 1610 | 58 | caccttccgacgclgtcta | 128 | tagacagcgtcggaaggtg |
| NM_001306068_1611_1629 | 1611 | 59 | accttccgacgctgtctag | 129 | ctagacagcgtcggaaggt |
| NM_001306068_1612_1630 | 1612 | 60 | ccttccgacgctgtctagg | 130 | cctagacagcgtcggaagg |
| NM_001306068_1613_1631 | 1613 | 61 | cttccgacgctgtctaggc | 131 | gcctagacagcgtcggaag |
| NM_001306068_1615_1633 | 1615 | 62 | tccgacgctgtctaggcaa | 132 | ttgcctagacagcgtcgga |
| NM_001306068_1616_1634 | 1616 | 63 | ccgacgctgtctaggcaaa | 133 | tttgcctagacagcgtcgg |
| NM_001306068_1619_1637 | 1619 | 64 | acgctgtctaggcaaacct | 134 | aggtttgcctagacagcgt |
| NM_001306068_1632_1650 | 1632 | 65 | aaacctggattagagttac | 135 | gtaactctaatccaggttt |
| NM_001306068_336_354 | 336 | 66 | ctttgagaaggatcgcttt | 136 | aaagcgatccttctcaaaa |
| NM_001306068_672_690 | 672 | 67 | gccggcagaggggatctcc | 137 | ggagatcccctctgccggc |
| NM_001306068_882_900 | 882 | 68 | gggccaaggggtgcttgcg | 138 | cgcaagcaccccttggccc |
| NM_001306068_884_902 | 884 | 69 | gccaaggggtgcttgcgcc | 139 | ggcgcaagcaccccttggc |
| NM_001306068_1045_1063 | 1045 | 70 | atgcaaggcatcccggcgc | 140 | gcgccgggatgccttgcat |

TABLE 12

| | 19 mer | SEQ | | SEQ | |
|---|---|---|---|---|---|
| | start | ID | sense/passenger_seq | ID | antisense/guide_seq |
| Name | site | NO | (5′-3′) | NO | (5′-3′) |
| NM_001306068_11_29 | 11 | 141 | csgsacacCfCfUfcg | 211 | UfsUfsgcuGfuccgagg |
| | | | gacagcsasa | | GfuGfucgsusu |
| NM_001306068_57_75 | 57 | 142 | ascsggcgAfCfGfg | 212 | UfsAfscgaGfucuccgu |
| | | | agacucgsusa | | CfgCfcgususu |
| NM_001306068_58_76 | 58 | 143 | csgsgcgaCfGfGfa | 213 | UfsAfsacgAfgucuccg |
| | | | gacucgususa | | UfcGfccgsusu |
| NM_001306068_59_77 | 59 | 144 | gsgscgacGfGfAfg | 214 | UfsAfsaacGfagucucc |
| | | | acucguususa | | GfuCfgccsusu |
| NM_001306068_60_78 | 60 | 145 | gscsgacgGfAfGfa | 215 | UfsCfsaaaCfgagucucC |
| | | | cucgauusgsa | | fgUfcgcsusu |
| NM_001306068_61_79 | 61 | 146 | csgsacggAfGfAf | 216 | UfsCfscaaAfgagucuC |
| | | | ucguuugsgsa | | fcGfucgsusu |
| NM_001306068_62_80 | 62 | 147 | gsascggaGfAfCfa | 217 | UfsUfsccaAfacgaguc |
| | | | cguuuggsasa | | UfcCfgucsusu |
| NM_001306068_63_81 | 63 | 148 | ascsggagAfCfUfc | 218 | UfsGfsuccAfaacgagu |
| | | | guuuggascsa | | CfuCfcgususu |
| NM_001306068_77_95 | 77 | 149 | gsgsacccCfGfAfgc | 219 | UfsCfsgcuUfuggcucg |
| | | | caaagcsgsa | | GfgGfuccsusu |
| NM_001306068_78_96 | 78 | 150 | gsasccccGfAfGfcc | 220 | UfsUfscgcUfuuggcuc |
| | | | aaagcgsasa | | GfgGfgucsusu |
| NM_001306068_79_97 | 79 | 151 | ascscccgAfGfCfca | 221 | UfsCfsucgCfuuuggcu |
| | | | aagcgasgsa | | CfgGfcgususu |
| NM_001306068_99_117 | 99 | 152 | cscsugcgAfGfCfc | 222 | UfsUfscaaAfgcaggcu |
| | | | ugcauugsasa | | CfgCfaggsusu |
| NM_001306068_102_120 | 102 | 153 | gscsgagcCfUfGf | 223 | UfsCfsgcuCfaaagcagG |
| | | | uuugagcsgsa | | fcUfcgcsusu |
| NM_001306068_137_155 | 137 | 154 | uscsgccaCCfAfga | 224 | UfsGfsccgUfucucugg |
| | | | gaacggscsa | | UfgGfcgasusu |
| NM_001306068_160_178 | 160 | 155 | csasggccAfUfCfg | 225 | UfsCfsggaAfugccgau |
| | | | gcauuccsgsa | | GfgCfcugsusu |
| NM_001306068_162_180 | 162 | 156 | gsgsccauCfGfGfca | 226 | UfsUfsccgGfaaugccg |
| | | | uuccggsasa | | AfuGfgccsusu |
| NM_001306068_163_181 | 163 | 157 | gscscaucGfGfCfau | 227 | UfsCfsuccGfgaaugcc |
| | | | uccggasgsa | | GfaUfggcsusu |
| NM_001306068_231_249 | 231 | 158 | gscsaccgCfCfCfg | 228 | UfsCfsgagAfuucccgc |
| | | | gaaucucsgsa | | CfgGfugcsusu |
| NM_001306068_232_250 | 232 | 159 | csasccggCfGfGfga | 229 | UfsCfscgaGfauucccgC |
| | | | aucucgsgsa | | fcGfgugsusu |
| NM_001306068_274_292 | 274 | 160 | cscsagaaGfGfCfcg | 230 | UfsCfsuuuCfgccggcc |
| | | | gcgaaasgsa | | UfuCfuggsusu |
| NM_001306068_276_294 | 276 | 161 | asgsaaggCfCfGfgc | 231 | UfsCfsgcuUfucgccgg |
| | | | gaaagcsgsa | | CfcUfucususu |
| NM_001306068_277_295 | 277 | 162 | gsasaggcCfGfGfc | 232 | UfsCfscgcUfuncgccg |
| | | | gaaagcgsgsa | | GfcCfuucsusu |
| NM_001306068_285_303 | 285 | 163 | gscsgaaaGfCfGfga | 233 | UfsAfscggCfgguccgc |
| | | | ccgccgsusa | | UfuUfcgcsusu |
| NM_001306068_287_305 | 287 | 164 | gsasaagcGfGfAfcc | 234 | UfsUfsgacGfgcggucc |
| | | | gccgucsasa | | GfcUfaucsusu |
| NM_001306068_292_310 | 292 | 165 | csgsgaccGfCfCfgu | 235 | UfsUfsccgGfugacggc |
| | | | caccggsasa | | GfgUfccgsusu |
| NM_001306068_293_311 | 293 | 166 | gsgsaccgCfCfGfuc | 236 | UfsAfsuccGfgugacgg |
| | | | accggasusa | | CfgGfuccsusu |
| NM_001306068_294_312 | 294 | 167 | gsasccgcCfGfUfca | 237 | UfsGfsaucCfggugacg |
| | | | ccggauscsa | | GfcGfgucsusu |
| NM_001306068_389_407 | 389 | 168 | asgsacggGfCfCfuc | 238 | UfsAfscucCfgggaggc |
| | | | ccggagsusa | | CfGfucususu |
| NM_001306068_524_542 | 524 | 169 | cscsucgaGfGfGfu | 239 | UfsCfsgaaGfgcgacccA |
| | | | cgccuucsgsa | | fcGfaggsusu |
| NM_001306068_525_543 | 525 | 170 | csuscgugGfGfUfc | 240 | UfsGfscgaAfggcgacc |
| | | | gccuucgscsa | | CfaCfgagsusu |
| NM_001306068_679_697 | 679 | 171 | gsasggggAfUfCfu | 241 | UfsAfsgguUfgggagau |
| | | | cccaaccsusa | | CfcCfcucsusu |
| NM_001306068_704_722 | 704 | 172 | csgscgcgGfGfGfa | 242 | UfsAfsggcGfaaaucccC |
| | | | uuucgccsusa | | fgCfgcgsusu |
| NM_001306068_705_723 | 705 | 173 | gscsgcggGfGfAfu | 243 | UfsUfsaggCfgaaauccC |
| | | | uucgccusasa | | fcGfcgcsusu |
| NM_001306068_708_726 | 708 | 174 | csgsgggaUfUfUfc | 244 | UfsGfscguAfggcgaaa |
| | | | gccuacgscsa | | UfcCfccgsusu |
| NM_001306068_893_911 | 893 | 175 | usgscuugCfGfCfc | 245 | UfsAfscguGfgguggcg |
| | | | acccacgsusa | | CfaAfgcasusu |
| NM_001306068_1132_1150 | 1132 | 176 | csusggcgAfGfCfc | 246 | UfsAfsaacUfccgggcu |
| | | | cggaguususa | | CfgCfcagsusu |
| NM_001306068_1134_1152 | 1134 | 177 | gsgscgagCfCfCfg | 247 | UfsAfsgaaAfcuccggg |
| | | | gaguuucsusa | | CfuCfgccsusu |
| NM_001306068_1158_1176 | 1158 | 178 | gsgscgcaAfCfCfuc | 248 | UfsUfscuaGfgagaggu |
| | | | uccuagsasa | | UfgCfgccsusu |
| NM_001306068_1159_1177 | 1159 | 179 | gscsgcaaCfCfUfcu | 249 | UfsUfsucuAfggagagg |
| | | | ccuagasasa | | UfuGfcgcsusu |
| NM_001306068_1163_1181 | 1163 | 180 | asasccucUfCfCfua | 250 | UfsCfscguUfucuagga |
| | | | gaaacgsgsa | | GfaGfguususu |
| NM_001306068_1236_1254 | 1236 | 181 | csasgcgaGfGfAfa | 251 | UfsCfsgguAfuucuucc |
| | | | gaauaccsgsa | | UfcGfcugsusu |
| NM_001306068_1237_1255 | 1237 | 182 | asgscgagGfAfAfg | 252 | UfsCfscggUfauucuuc |
| | | | aauaccgsgsa | | CfuCfgcususu |
| NM_001306068_1238_1256 | 1238 | 183 | gscsgaggAfAfGfa | 253 | UfsCfsccgGfuauucuu |
| | | | auaccggsgsa | | CfcUfcgcsusu |
| NM_001306068_1284_1302 | 1284 | 184 | gsusugggAfCfGfg | 254 | UfsAfscccGfaccccguC |
| | | | ggucgggsusa | | fcCfaacsusu |
| NM_001306068_1290_1308 | 1290 | 185 | ascsggggUfCfGfg | 255 | UfsCfsgaaCfcacccgaC |
| | | | gugguucsgsa | | fcCfcgususu |
| NM_001306068_1294_1312 | 1294 | 186 | gsgsucggGfUfGfg | 256 | UfsGfscccCfgaaccacC |
| | | | uucggggscsa | | fcGfaccsusu |
| NM_001306068_1295_1313 | 1295 | 187 | gsuscgggUfGfGfu | 257 | UfsUfsgccCfcgaaccaC |
| | | | ucggggcsasa | | fcCfgacsusu |
| NM_001306068_1315_1333 | 1315 | 188 | gscsggugGfCfCfu | 258 | UfsCfsgaaAfgagaggc |
| | | | cucuuucsgsa | | CfaCfcgcsusu |
| NM_001306068_1316_1334 | 1316 | 189 | csgsguggCfCfUfc | 259 | UfsGfscgaAfagagagg |
| | | | ucuuucgscsa | | CfAfccgsusu |
| NM_001306068_1317_1335 | 1317 | 190 | gsgsuggcCfUfCfu | 260 | UfsCfsgcgAfaagagag |
| | | | cuuucgcsgsa | | GfcCfaccsusu |
| NM_001306068_1321_1339 | 1321 | 191 | gscscucuCfUfUfu | 261 | UfsUfscccCfgcgaaagA |
| | | | cgcggggsasa | | fgAfggcsusu |
| NM_001306068_1340_1358 | 1340 | 192 | csasccugGfCfUfg | 262 | UfsUfsccgUfagccagcC |
| | | | gcuacggsasa | | faGfgugsusu |
| NM_001306068_1350_1368 | 1350 | 193 | gscsuacgGfAfGfg | 263 | UfsGfsacaCfgccccucC |
| | | | ggcguguscsa | | fgUfagcsusu |
| NM_001306068_1351_1369 | 1351 | 194 | csusacggAfGfGfg | 264 | UfsAfsgacAfcgccccuC |
| | | | gcgugucsusa | | fcGfuagsusu |
| NM_001306068_1539_1557 | 1539 | 195 | ascsgugcAfAfGfg | 265 | UfsGfscgaGfcucccuu |
| | | | gagcucgscsa | | GfAfcgususu |
| NM_001306068_1540_1558 | 1540 | 196 | csgsugcaAfGfGfg | 266 | UfsAfsgcgAfgcucccu |
| | | | agcucgcsusa | | UfgCfacgsusu |
| NM_001306068_1541_1559 | 1541 | 197 | gsusgcaaGfGfGfa | 267 | UfsCfsagcGfagcucccU |
| | | | gcucgcusgsa | | fuGfcacsusu |
| NM_001306068_1610_1628 | 1610 | 198 | csasccuuCfCfGfac | 268 | UfsAfsgacAfgcgucgg |
| | | | gcugucsusa | | AfaGfgugsusu |
| NM_001306068_1611_1629 | 1611 | 199 | ascscaucCfGfAfcg | 269 | UfsUfsagaCfagcgucg |
| | | | cugucusasa | | GfaAfggususu |
| NM_001306068_1612_1630 | 1612 | 200 | cscsuuccGfAfCfgc | 270 | UfsCfsuagAfcagcguc |
| | | | ugucuasgsa | | GfgAfaggsusu |
| NM_001306068_1613_1631 | 1613 | 201 | csusuccgAfCfGfc | 271 | UfsCfscuaGfacagcguC |
| | | | ugucuagsgsa | | fgGfaagsusu |
| NM_001306068_1615_1633 | 1615 | 202 | uscscgacGfCfUfg | 272 | UfsUfsgccUfagacagc |
| | | | ucuaggcsasa | | GfuCfggasusu |
| NM_001306068_1616_1634 | 1616 | 203 | cscsgacgCfUfGfuc | 273 | UfsUfsugcCfuagacag |
| | | | uaggcasasa | | CfgUfcggsusu |
| NM_001306068_1619_1637 | 1619 | 204 | ascsgcugUfCfUfa | 274 | UfsGfsguuUfgccuaga |
| | | | ggcaaacscsa | | CfaGfcgususu |
| NM_001306068_1632_1650 | 1632 | 205 | asasaccuGfGfAfu | 275 | UfsUfsaacUfcuaauccA |
| | | | uagaguusasa | | fgGfuuususu |
| NM_001306068_336_354 | 336 | 206 | csusuugaGfAfAfg | 276 | UfsAfsagcGfauccuuc |
| | | | gaucgcususa | | UfcAfaagsusu |
| NM_001306068_672_690 | 672 | 207 | gscscggcAfGfAfg | 277 | UfsGfsagaUfccccucu |
| | | | gggaucuscsa | | GfcCfggcsusu |
| NM_001306068_882_900 | 882 | 208 | gsgsgccaAfGfGfg | 278 | UfsGfscaaGfcaccccuU |
| | | | gugcuugscsa | | fgGfcccsusu |
| NM_001306068_884_902 | 884 | 209 | gscscaagGfGfGfu | 279 | UfsGfscgcAfagcacccC |
| | | | gcuugcgscsa | | fuUfggcsusu |
| NM_001306068_1045_1063 | 1045 | 210 | asusgcaaGfGfCfau | 280 | UfsCfsgccGfggaugcc |
| | | | cccggcsgsa | | UfuGfcaususu |

vpN = vinyl phosphonate 2′-MOE; upper case (N) = 2′-OH (ribo); lower case (n) = 2′-O-Me(methyl) dN = 2′-H (deoxy); Nf = 2′-F (fluoro); s = phosphorothioate backbone modification; iB = inverted abasic

TABLE 13

| SEQ ID | sense/passenger_seq | SEQ ID | antisense/guide_seq |
|---|---|---|---|
| NO | (5′-3′) | NO | (5′-3′) |
| 371 | CTGCCTCTCCACCAGCCCA | 372 | TGGGCTGGTGGAGAGGCAG |
| 373 | GCAGAGATGGAGAGAGGAA | 374 | TTCCTCTCTCCATCTCTGC |
| 375 | GCGGTTTCCTCCGGGACAA | 376 | TTGTCCCGGAGGAAACCGC |
| 377 | GGACGACGGAGGCGTGATT | 378 | AATCACGCCTCCGTCGTCC |
| 379 | CGGGCACCCGGAAACTGCAGG | 380 | TTCCCTGCATGTTTCCGGGTGCCC |
| | GAA | | G |
| 381 | CCGGAAACATGCAGGGAAG | 382 | CTTCCCTGCATGTTTCCGG |
| 383 | GAAATGAACGAGAGCCACA | 384 | TGTGGCTCTCGTTCATTTC |
| 385 | TGGCACACTCAAGACTCCCACGG | 386 | CTCCGTGGGAGTCTTGAGTGTGC |
| | AG | | CA |
| 387 | CCACGGAGGTTCAGTTCCA | 388 | TGGAACTGAACCTCCGTGG |
| 389 | ACCACCACCACCACCACCA | 390 | TGGTGGTGGTGGTGGTGGT |
| 391 | CGCCATTCATGAAGGGGTG | 392 | CACCCCTTCATGAATGGCG |
| 393 | CATGAAGGGGTGGAGCCTG | 394 | CAGGCTCCACCCCTTCATG |
| 395 | GAGCCTGCTTTGAGCGGAA | 396 | TTCCGCTCAAGCAGGCTC |
| 397 | CCGAGCCTTTGAGAAGGATCGCT | 398 | AAAGCGATCCTTCTCAAAGGCTC |
| | TT | | GG |
| 399 | GGCAGGGCGCCCGCGCAGG | 400 | CCTGCGCGGGCGCCCTGCC |
| 401 | GATGATTAGTTCAGAGATA | 402 | TATCTCTGAACTAATCATC |

TABLE 14

| | 19mer | SEQ | | | |
|---|---|---|---|---|---|
| | start | ID | sense/passenger_seq | SEQ | antisense/guide_seq |
| Name | site | NO | (5′-3′) | ID NO | (5′-3′) |
| NM_001306068_57_75 | 57 | 142 | ascsggcgAfCfGfgag | 412 | usAfscgaGfucuccguC |
| | | | acucgsusa | | fgCfcgususu |
| NM_001306068_61_79 | 61 | 146 | csgsacggAfGfAfcuc | 413 | usCfscaaAfcgagucuC |
| | | | gauugsgsa | | fcGfucgsusu |
| NM_001306068_336_354 | 336 | 206 | csusuugaGfAfAfgga | 414 | usAfsagcGfauccuucU |
| | | | ucgcususa | | fAfaagsusu |
| NM_001306068_1540_1558 | 1540 | 196 | csgsugcaAfGfGfgag | 415 | usAfsgCgAfgcucccuU |
| | | | cucgcsusa | | fgCfacgsusu |
| NM_001306068_1613_1631 | 1613 | 201 | csusuccgAfCfGfug | 416 | usCfscuaGfacagcguC |
| | | | ucuagsgsa | | fgGfaagsusu |
| NM_001306068_1615_1633 | 1615 | 202 | usCscgacGfCfUfguc | 417 | usUfsgccUfagacagcG |
| | | | uaggcsasa | | fuCfggasusu |
| NM_001306068_1616_1634 | 1616 | 203 | cscsgacgCfUfGfucu | 418 | asUfsugcCfuagacagC |
| | | | aggcasasa | | fgUfcggsusu |
| NM_001306068_1619_1637 | 1619 | 204 | ascsgcugUfCfUfagg | 419 | usGfsguuUfgccuagaC |
| | | | caaacscsa | | faGfgususu |
| NM_001306068_1632_1650 | 1632 | 205 | asasaccuGfGfAfuua | 420 | usUfsaacUfcuaauccA |
| | | | gaguusasa | | fgGfuuususu |

vpN = vinyl phosphonate vpUq; upper case (N) = 2′-OH (ribo); lower case (n) = 2′-O-Me(methyl) dN = 2′-H (deoxy); Nf = 2′F (fluoro); s = phosphorothioate backbone modification; iB = inverted abasic

TABLE 15

| | 19mer | SEQ | | | |
|---|---|---|---|---|---|
| | start | ID | sense/passenger_seq | SEQ | antisense/guide_seq |
| Name | site | NO | (5′-3′) | ID NO | (5′-3′) |
| NM_001306068_57_75 | 57 | 142 | ascsggcgAfCfGfgagac | 430 | vpUsAfscgaGfucaccguCf |
| | | | ucgsusa | | gCfcgususu |
| NM_001306068_61_79 | 61 | 146 | csgsacggAfGfAfcucgu | 431 | vpUsCfscaaAfgagucuCf |
| | | | uugsgsa | | cGfucgsusu |
| NM_001306068_336_354 | 336 | 206 | csusuugaGfAfAfggauc | 432 | vpUsAfsagcGfauccuucUf |
| | | | gcususa | | cAfaagsusu |
| NM_001306068_1540_1558 | 1540 | 196 | csgsugcaAfGfGfgagcu | 433 | vpUsAfsgcgAfgcucccuUf |
| | | | cgcsusa | | gCfacgsusu |
| NM_001306068_1613_1631 | 1613 | 201 | csusuccgAfCfGfcuguc | 434 | vpUsCfscuaGfacagcguCf |
| | | | uagsgsa | | gGfaagsusu |
| NM_001306068_1615_1633 | 1615 | 202 | uscscgacGfCfUfgucua | 435 | vpUsUfsgccUfagacagcGf |
| | | | ggcsasa | | aCfggasasu |
| NM_001306068_1616_1634 | 1616 | 203 | cscsgacgCfUfGfucuag | 436 | vpUsUfsugcCfuagacagCf |
| | | | gcasasa | | gUfcggsusu |
| NM_001306068_1619_1637 | 1619 | 204 | ascsgcugUfCfUfaggca | 437 | vpUsGfsgauUfgccuagaCf |
| | | | aacscsa | | aGfcgususu |
| NM_001306068_16321650 | 1632 | 205 | asasaccuGfGfAfuuaga | 438 | vpUsUfsaacUfcuaauccAf |
| | | | guusasa | | gGfuuususu |

vpN = vinyl phosphonate vpUq; upper case (N) = 2′-OH (ribo); lower case (n) = 2′-O-Me (methyl) dN = 2′-H (deoxy); Nf = 2′F (fluoro); s = phosphorothioate backbone modification; iB = inverted abasic

### Example 2. siRNA Sequences and Synthesis

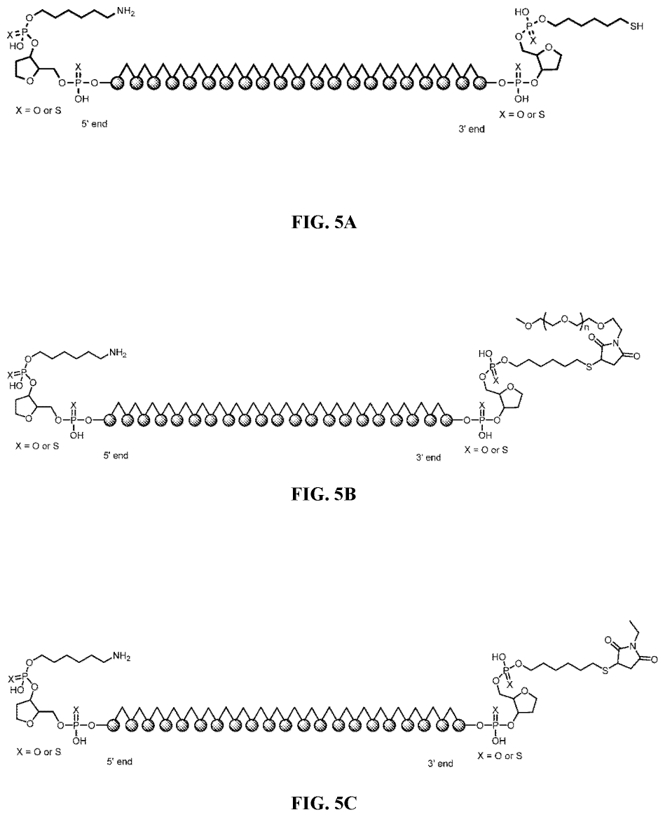

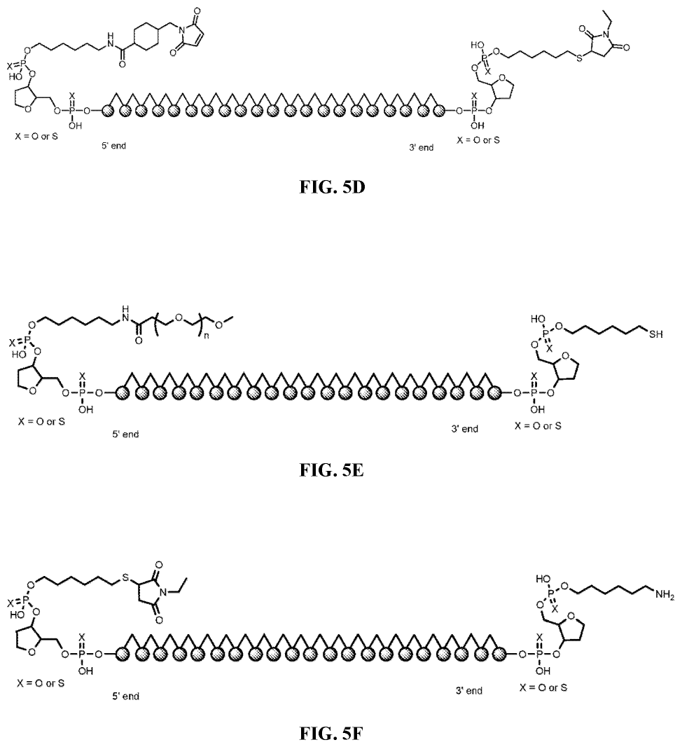

All siRNA single strands were fully assembled on solid phase using standard phosphoramidite chemistry and purified over HPLC. Purified single strands were duplexed to get the double stranded siRNA. For vinylphosphonate modified guide strand, the guide strand was produced with a vinylphosphonate modified nucleotide structures at the 5′ end (VpUq). All the siRNA passenger strand contains conjugation handles in different formats, C₆—NH₂ and/or C₆—SH, one at each end of the strand. The conjugation handle or handles were connected to the siRNA passenger strand or siRNA guide strand via inverted abasic phosphodiester or phosphorothioate. FIGS. 5A-5F are representative structures of the formats used in the in vivo experiments. FIG. 5A illustrates a representative structure of siRNA with C₆—NH₂ conjugation handle at the 5′ end and C₆—SH at 3′end of the passenger strand or guide strand. FIG. 5B illustrates a representative structure of siRNA passenger strand or guide strand with C₆-NH2 conjugation handle at the 5′ end and C₆—S-PEG at 3′ end. FIG. 5C illustrates a representative structure of siRNA passenger strand or guide strand with C₆—NH₂ conjugation handle at the 5′ end and C₆—S-NEM at 3′ end. FIG. 5D illustrates a representative structure of siRNA passenger strand with C₆-N-SMCC conjugation handle at the 5′ end and C₆—S-NEM at 3′ end. FIG. 5E illustrates a representative structure of siRNA passenger strand or guide strand with PEG at the 5′ end and C₆—SH at 3′ end. FIG. 5F illustrates a representative structure of siRNA passenger strand or guide strand with C₆-S-NEM at the 5′ end and C₆—NH₂ conjugation handle at 3′ end.

### Example 3. Conjugate Synthesis

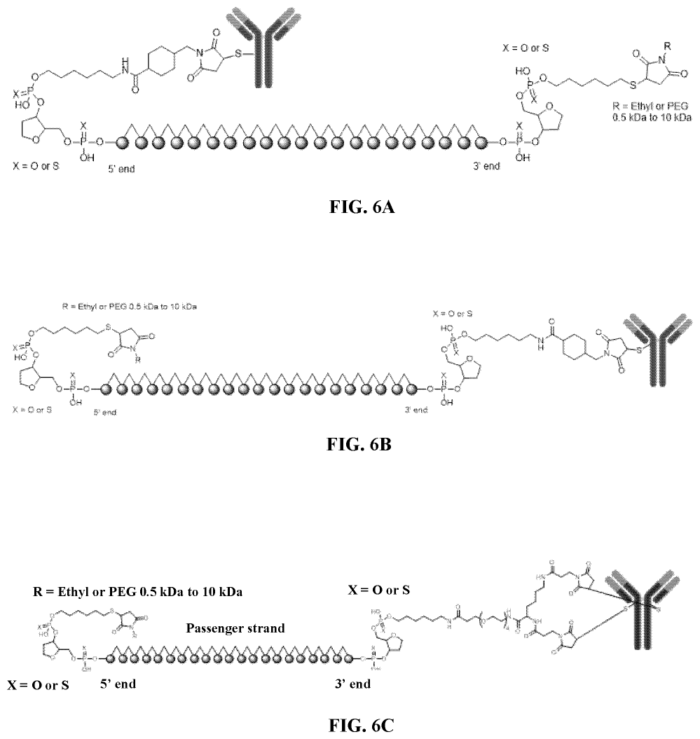

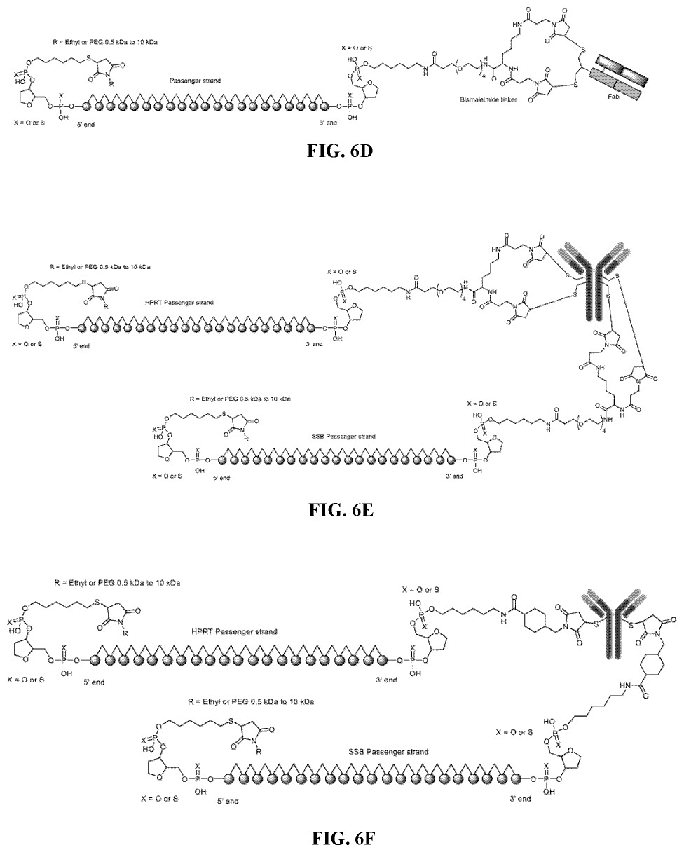

FIG. 6A-FIG. 6F illustrate exemplary structure of A-X₁-B-X₂-Y (Formula I) architectures described herein. FIG. 6A illustrates an antibody-Cys-SMCC-5′-passenger strand (Architecture-1). This conjugate was generated by antibody inter-chain cysteine conjugation to maleimide (SMCC) at the 5′ end of passenger strand. FIG. 6B illustrates an antibody-Cys-SMCC-3′-Passenger strand (Architecture-2). This conjugate was generated by antibody inter-chain cysteine conjugation to maleimide (SMCC) atthe 3′ end of passenger strand. FIG. 6C illustrates an antibody-Cys-bisMal-3′-Passenger strand (ASC Architecture-3). This conjugate was generated by antibody inter-chain cysteine conjugation to bismaleimide (bisMal)linker at the 3′ end of passenger strand. FIG. 6D illustrates a model structure of the Fab-Cys-bisMal-3′-Passenger strand (ASC Architecture-4). This conjugate was generated by Fab inter-chain cysteine conjugation to bismaleimide (bisMal) linker at the 3′ end of passenger strand. FIG. 6E illustrates a model structure of the antibody siRNA conjugate with two different siRNAs attached to one antibody molecule (ASC Architecture-5). This conjugate was generated by conjugating a mixture of SSB and HPRT siRNAs to the reduced mAb inter-chain cysteines to bismaleimide (bisMal) linker at the 3′ end of passenger strand of each siRNA. FIG. 6F illustrates a model structure of the antibody siRNA conjugate with two different siRNAs attached (ASC Architecture-6). This conjugate was generated by conjugating a mixture of SSB and HPRT siRNAs to the reduced mAb inter-chain cysteines to maleimide (SMCC) linker at the 3′ end of passenger strand of each siRNA.

### Example 3.1 Antibody siRNA Conjugate Synthesis Using SMCC Linker

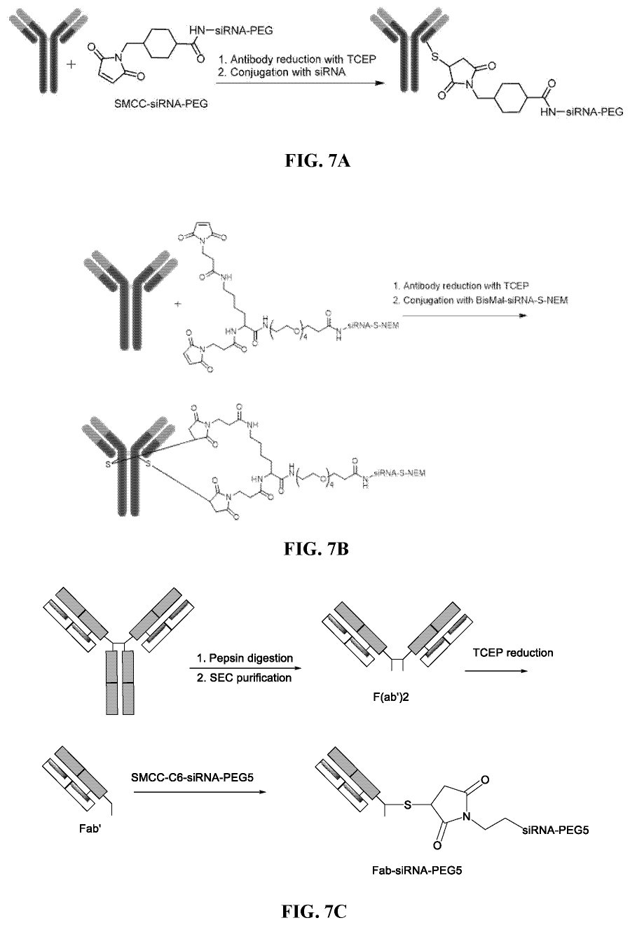

FIG. 7A illustrates an exemplary synthesis scheme (Synthesis scheme-1) for antibody-Cys-SMCC-siRNA-PEG conjugates via antibody cysteine conjugation.

Step 1: Antibody Interchain Disulfide Reduction with TCEP

Antibody was buffer exchanged with borax buffer (pH 8) and made up to 10 mg/ml concentration. To this solution, 2 equivalents of TCEP in water was added and rotated for 2 hours at RT. The resultant reaction mixture was buffer exchanged with pH 7.4 PBS containing 5 mM EDTA and added to a solution of SMCC-C6-siRNA or SMCC-C6-siRNA-C6-NHCO-PEG-XkDa (2 equivalents) (X=0.5 kDa to 10 kDa) in pH 7.4 PBS containing 5 mM EDTA at RT and rotated overnight. Analysis of the reaction mixture by analytical SAX column chromatography showed antibody siRNA conjugate along with unreacted antibody and siRNA.

### Step 2: Purification

The crude reaction mixture was purified by AKTA explorer FPLC using anion exchange chromatography method-1 as described in Example 3.4. Fractions containing DAR1 and DAR>2 antibody-siRNA-PEG conjugates were separated, concentrated and buffer exchanged with pH 7.4 PBS.

### Step 3: Analysis of the Purified Conjugate

The isolated conjugates were characterized by SEC, SAX chromatography and SDS-PAGE. The purity of the conjugate was assessed by analytical HPLC using either anion exchange chromatography method-2 or anion exchange chromatography method-3. Both methods are described in Example 3.4. Isolated DAR1 conjugates are typically eluted at 9.0 0.3 min on analytical SAX method and are greater than 90% pure. The typical DAR>2 cysteine conjugate contains more than 85% DAR2 and less than 15% DAR3.

### Example 3.2. Antibody siRNA Conjugate Synthesis Using Bis-Maleimide (BisMal) Linker

FIG. 7B illustrates an exemplary synthesis scheme (Synthesis scheme-2) for antibody-Cys-BisMal-siRNA-PEG conjugates.

Step 1: Antibody reduction with TCEP

Antibody was buffer exchanged with borax buffer (pH 8) and made up to 5 mg/ml concentration. To this solution, 2 equivalents of TCEP in water was added and rotated for 2 hours atRT. The resultant reaction mixture was exchanged with pH 7.4 PBS containing 5 mM EDTA and added to a solution of BisMal-C6-siRNA-C6-S-NEM (2 equivalents) in pH 7.4 PBS containing 5 mM EDTA at RT and kept at 4° C. overnight. Analysis of the reaction mixture by analytical SAX column chromatography showed antibody siRNA conjugate along with unreacted antibody and siRNA.

### Step 2: Purification

The crude reaction mixture was purified by AKTA explorer FPLC using anion exchange chromatography method-1. Fractions containing DAR1 and DAR2 antibody-siRNA conjugates were separated, concentrated and buffer exchanged with pH 7.4 PBS.

### Step-3: Analysis of the Purified Conjugate

The isolated conjugates were characterized by either mass spec or SDS-PAGE. The purity of the conjugate was assessed by analytical HPLC using either anion exchange chromatography method-2 or 3 as well as size exclusion chromatography method-1.

### Example 3.3. Fab′ Generation from mAb and Conjugation to siRNA

FIG. 7C illustrates an exemplary synthesis scheme (Synthesis scheme-3) for Fab-siRNA conjugate generation.

Step 1: Antibody Digestion with Pepsin

Antibody was buffer exchanged with pH 4.0, 20 mM sodium acetate/acetic acid buffer and made up to 5 mg/ml concentration. Immobilized pepsin (Thermo Scientific, Prod #20343) was added and incubated for 3 hours at 37° C. The reaction mixture was filtered using 30 kDa MWCO Amicon spin filters and pH 7.4 PBS. The retentate was collected and purified using size exclusion chromatography to isolate F(ab′)2. The collected F(ab′)2 was then reduced by 10 equivalents of TCEP and conjugated with SMCC-C₆-siRNA-PEG5 at room temperature in pH 7.4 PBS. Analysis of reaction mixture on SAX chromatography showed Fab-siRNA conjugate along with unreacted Fab and siRNA-PEG.

### Step 2: Purification

The crude reaction mixture was purified by AKTA explorer FPLC using anion exchange chromatography method-1. Fractions containing DAR1 and DAR2 Fab-siRNA conjugates were separated, concentrated and buffer exchanged with pH 7.4 PBS.

### Step-3: Analysis of the Purified Conjugate

The characterization and purity of the isolated conjugate was assessed by analytical HPLC using anion exchange chromatography method-2 or 3 as well as by SEC method-1.

### Example 3.4. Purification and Analytical Methods

Anion exchange chromatography method (SAX)-1.
1. Column: Tosoh Bioscience, TSKGel SuperQ-5PW, 21.5 mm ID X 15 cm, 13 μm
2. Solvent A: 20 mM TRIS buffer, pH 8.0; Solvent B: 20 mM TRIS, 1.5 M NaCl, pH 8.0; Flow Rate: 6.0 ml/min
3. Gradient:

Gradient:

| a. | % A | % B | Column Volume |
|---|---|---|---|
| b. | 100 | 0 | 1.00 |
| c. | 60 | 40 | 18.00 |
| d. | 40 | 60 | 2.00 |
| e. | 40 | 60 | 5.00 |
| f. | 0 | 100 | 2.00 |
| g. | 100 | 0 | 2.00 |

Anion exchange chromatography (SAX) method-2

1. Column: Thermo Scientific, ProPac™ SAX-10, Bio LC™, 4×250 mm

2. Solvent A: 80% 10 mM TRIS pH 8, 20% ethanol; Solvent B: 80% 10 mM TRIS pH 8, 20% ethanol, 1.5 M NaCl; Flow Rate: 0.75 ml/min

3.

Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 13.00 | 40 | 60 |
| f. | 15.00 | 90 | 10 |
| g. | 20.00 | 90 | 10 |

Anion exchange chromatography (SAX) method-3
1. Column: Thermo Scientific, ProPac™ SAX-10, Bio LC™, 4×250 mm
2. Solvent A: 80% 10 mM TRIS pH 8, 20% ethanol; Solvent B: 80% 10 mM TRIS pH 8, 20% ethanol, 1.5 M NaCl
3. Flow Rate: 0.75 ml/min
4. Gradient:

Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 23.00 | 40 | 60 |
| f. | 25.00 | 90 | 10 |
| g. | 30.00 | 90 | 10 |

### Size Exclusion Chromatography (SEC) Method-1

1. Column: TOSOH Biosciences, TSKgelG3000SW XL, 7.8×300 mm, 5 μM
2. Mobile phase: 150 mM phosphate buffer
3. Flow Rate: 1.0 ml/min for 15 mins
   Example 3.5. Antibody siRNA Conjugate Synthesis using bis-maleimide (BisMal) linker
   Antibody reduction with TCEP

Antibody was buffer exchanged with 25mMborate buffer (pH 8) with 1 mM DTPA and made up to IOmg/ml concentration. To this solution, 4 equivalents of TCEP in the same borate buffer were added and incubated for 2 hours at 37° C. The resultant reaction mixture was combined with a solution of BisMal-siRNA (1.25 equivalents) in pH 6.0 10 mM acetate buffer at RT and kept at 4° C. overnight. Analysis of the reaction mixture by analytical SAX column chromatography showed antibody siRNA conjugate along with unreacted antibody and siRNA. The reaction mixture was treated with 10 EQ of N-ethylmaleimide (in DMSO at 10 mg/mL) to cap any remaining free cysteine residues.

### Step 2: Purification

The crude reaction mixture was purified by AKTA Pure FPLC using anion exchange chromatography (SAX) method-1. Fractions containing DAR1 and DAR2 antibody-siRNA conjugates were isolated, concentrated and buffer exchanged with pH 7.4 PBS.

### Anion Exchange Chromatography Method (SAX)-1.

Column: Tosoh Bioscience, TSKGel SuperQ-5PW, 21.5 mm ID X 15 cm, 13 um

Solvent A: 20 mM TRIS buffer, pH 8.0; Solvent B: 20 mM TRIS, 1.5 M NaCl, pH 8.0; Flow Rate: 6.0 ml/min

Gradient:

| a. | % A | % B | Column Volume |
|---|---|---|---|
| b. | 100 | 0 | 1 |
| c. | 81 | 19 | 0.5 |
| d. | 50 | 50 | 13 |
| e. | 40 | 60 | 0.5 |
| f. | 0 | 100 | 0.5 |
| g. | 100 | 0 | 2 |

### Anion Exchange Chromatography (SAX) Method-2

Column: Thermo Scientific, ProPac™ SAX-10, Bio LC™, 4×250 mm

Solvent A: 80% 10 mM TRIS pH 8, 20% ethanol; Solvent B: 80% 10 mM TRIS pH 8, 20% ethanol, 1.5 M NaCl; Flow Rate: 0.75 ml/min

Gradient:

| a. | Time | % A | % B |
|---|---|---|---|
| b. | 0.0 | 90 | 10 |
| c. | 3.00 | 90 | 10 |
| d. | 11.00 | 40 | 60 |
| e. | 14.00 | 40 | 60 |
| f. | 15.00 | 20 | 80 |
| g. | 16.00 | 90 | 10 |
| h. | 20.00 | 90 | 10 |

### Example 4. In Vivo Activity of DUX4-Targeted AOCs in the FSHD Mouse Model ACTA1-MCM:FLExDUX4

The DUX4 siRNAs (DUX4.61 non-VP, DUX4.61 vpUq, and DUX4.1613 vpUq) were conjugated to the murine transferrin receptor (Tfrc) antibody to generate mouse-specific DUX4 AOCs. DUX4 AOCs were administered intravenously in the ACTA1-MCM:FLExDUX4 mouse model of FSHD disease that expresses human DUX4 gene: STOCK Tg(ACTA1-cre/Esr1*)2Kesr/J (Stock #025750) crossed with B6 (Cg)-Gt (ROSA)26Sortml.1(DUX4*)Plj/J (Stock #028710) (Jones T, Jones PL. A cre-inducible DUX4 transgenic mouse model for investigatingfacioscapulohumeral muscular dystrophy. PLoS One. 2018 Feb. 7; 13(2):e0192657). Age of mice at Day 0: 8-11 weeks (N=8 or 10 mixed males and females). Skeletal muscles were collected 3 weeks post single IV dose of DUX4 AOCs.

Gene expression was analyzed by RT-qPCR. Muscle tissue was homogenized in Trizol in Lysing Matrix D using homogenizer FastPrep-24 (MPBio) and spun 6,000 RPM for 5 mins at 4° C. RNA was isolated using Zymo-Spin™ I-96 kit according to manufacturer's instructions. cDNA was synthetized using High-Capacity cDNA Reverse Transcription Kit (Applied Biosystems) using SimpliAmp Thermal Cycler (Applied Biosystems). cDNA was analyzed by qPCR using TaqMan Fast Universal Master Mix II (Thermo Fisher) and TaqMan probes (Thermo Fisher) in duplicates, using QuantStudio 6 or 7 Flex Real-Time PCR instruments (Applied Biosystems). Data were analyzed by QuantStudio™ Real-Time PCR Software v1.3 (Applied Biosystems). The expression levels of 4 DUX4-target genes were evaluated: WFDC3, ILVBL, SLC15A2, SORD (Jones TI, Chew GL, Barraza-Flores P, Schreier S, Ramirez M, Wuebbles RD, Burkin DJ, Bradley RK, Jones PL. Transgenic mice expressing tunable levels of DUX4 develop characteristic facioscapulohurneral muscular dystrophy-like pathophysiology ranging in severity. Skelet Muscle. 2020 Apr. 11; 10(1):8). DUX4-targetgene expression was normalized to PPIB reference gene. The level of target mRNA downregulation was determined relative to PBS vehicle treated animals by using the 2^{-ΔΔCt} method.

General primer and TaqMan probe designs as well as the methodology for the stem-loop RT-qPCR (SL-RT-qPCR) assay have been described previously (Chen, 2005, Real-time quantification of microRNAs by stem-loop RT-PCR. Nucleic Acids Res 33, e179). A specific SL-RT-qPCR assay was designed to quantify the guide strand of the DUX4 siRNAs. Tissue homogenates were diluted into TE Buffer with 0.1% Triton X-100 and then analyzed by SL-RT-qPCR. Standard curves were generated by spiking different concentrations of siRNA into the appropriate matrix for comparison to the samples. Linear regressions of siRNA standard curves were performed in Prism and the slope and y-intercept values were used to interpolate tissue and plasma sample concentrations.

The composite of the DUX4 target genes is the geometric mean of 4 DUX4-target mouse genes (WFDC3, ILVBL, SLC15A2, and SORD) and the data is expressed as mean−/+SEM % of vehicle (PBS) treated animals, N=8 or 10 mixed males and females.

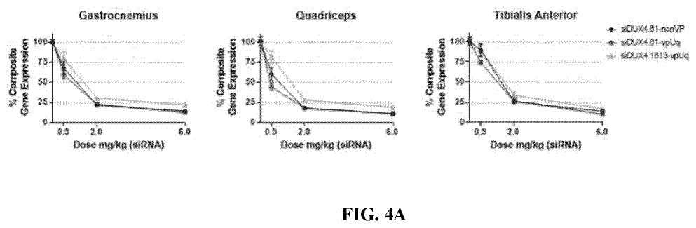

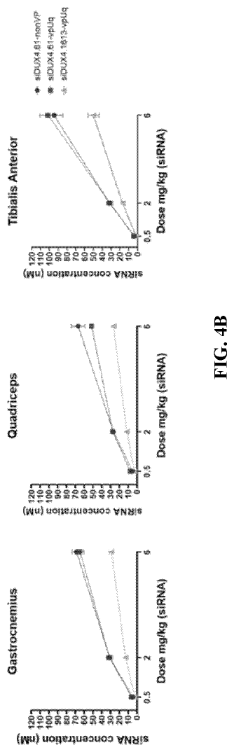

FIG. 4A and FIG. 4B show the in vivo activity of DUX4-targeted AOCs in the FSHD mouse model ACTA1-MCM;FLExDUX4. FIG. 4A shows DUX4 AOCs demonstrate a dose-dependent downregulation of the composite murine DUX4 target genes (WFDC3, ILVBL, SLC15A2, and SORD) in tibialis anterior, gastrocnemius and quadriceps skeletal muscles 3 weeks after the single AOC dose. In addition, FIG. 4B shows the dose dependent increase concentration for the DUX4 siRNAs in muscle tissue 3 weeks after the single intravenous dose of the DUX4 AOCs.

Overall, these data demonstrate a robust and durable activity of the DUX4 AOCs in vivo, thus demonstrating their potential treatment of FSHD disease.

### Example 5. Functional Improvement in Mouse Model of FSHD after Treatment with DUX4-Targeted AOCs

Example 5 demonstrates the efficacy of DUX4 siRNAs in suppression of FSHD disease phenotype after the treatment of DUX4-targeted AOCs in mice expressing human DUX4 in skeletal muscles. ACTA1-MCM;FLExDUX4 mice are treated at 6-9 weeks of age. 10 mice per group are treated with a single IP injection of Tamoxifen (TMX) 5 mg/kg either once or twice to induce FSHD phenotype. Within two days after the TMX dosing, mice are dosed by IV injection with test DUX4 AOCs and control AOC articles or with a vehicle. Immediately after the dosing and three times per week afterwards, mice are observed for the following: signs of pain, impaired locomotion, avoidance, hydration. Body weights are measured three times per week. Neuroscoring is performed three times per week.

The following functional measurements are performed to assess the muscle phenotype:
1. in vivo muscle force measurement (Isometric force-frequency curve and relaxation time from tetanus) 13 days post AOC treatment,
2. treadmill exercise at days 7, 10 and 14 post AOC treatment,
3. EMG 15 days post AOC treatment will be performed on all mice.

One or two days after the functional endpoint measurements are completed, mice are sacrificed and the following tissues necropsies are collected for further evaluation:
a. Gastrocnemius
   i. Left leg muscle is flash frozen and stored at −80° C.
   ii. Right leg muscle is fixed in 10% NBF at room temperature.
b. Tibialis Anterior
   i. Left leg muscle is flash frozen and stored at −80° C.
   ii. Right leg muscle is fixed in 10% NBF at room temperature.
c. Quadriceps
   i. Left leg muscle is flash frozen and stored at −80° C.
   ii. Right leg muscle is fixed in 10% NBF at room temperature.
d. Diaphragm cut in half
   i. Left half is flash frozen and stored at −80° C.
   ii. Right half is fixed in 10% NBF at room temperature.

Frozen tissue samples are analyzed for the DUX4-dependent gene expression and DUX4 siRNA concentration in the tissue.

The formaldehyde fixed tissues are trimmed for embedding. Two sections are cut from each tissue.
a. One section is stained with Sirius Red. On Sirius Red stained sections, the extent of fibrosis is measured by semiautomated image analysis.
b. The other section is stained for Reticulin. On Reticulin stained sections, muscle fiber sizes and % central nuclei are measured by automated image analysis.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A double-stranded polynucleic acid molecule that mediates RNA interference against DUX4, wherein the double-stranded polynucleic acid molecule comprises a sense strand and an antisense strand, wherein the antisense strand comprises 19 consecutive nucleotides from 5′ end of a nucleic acid sequence selected from SEQ ID NO: 412-420.

2. The double-stranded polynucleic acid molecule of claim 1, wherein the sense strand comprises 18 consecutive nucleotides from 5′ end of a nucleic acid sequence selected from SEQ ID NO: 142, 146, 196, or 201-206.

3. The double-stranded polynucleic acid molecule of claim 1,
wherein the antisense strand comprises 19 consecutive nucleotides from 5′ end of SEQ ID NO: 412 and the sense strand comprises 18 consecutive nucleotides from 5′ end of SEQ ID NO: 142;
wherein the antisense strand comprises 19 consecutive nucleotides from 5′ end of SEQ ID NO: 413 and the sense strand comprises 18 consecutive nucleotides from 5′ end of SEQ ID NO: 146;
wherein the antisense strand comprises 19 consecutive nucleotides from 5′ end of SEQ ID NO: 414 and the sense strand comprises 18 consecutive nucleotides from 5′ end of SEQ ID NO: 206;
wherein the antisense strand comprises 19 consecutive nucleotides from 5′ end of SEQ ID NO: 415 and the sense strand comprises 18 consecutive nucleotides from 5′ end of SEQ ID NO: 196;
wherein the antisense strand comprises 19 consecutive nucleotides from 5′ end of SEQ ID NO: 416 and the sense strand comprises 18 consecutive nucleotides from 5′ end of SEQ ID NO: 201;
wherein the antisense strand comprises 19 consecutive nucleotides from 5′ end of SEQ ID NO: 417 and the sense strand comprises 18 consecutive nucleotides from 5′ end of SEQ ID NO: 202;
wherein the antisense strand comprises 19 consecutive nucleotides from 5′ end of SEQ ID NO: 418 and the sense strand comprises 18 consecutive nucleotides from 5′ end of SEQ ID NO: 203;
wherein the antisense strand comprises 19 consecutive nucleotides from 5′ end of SEQ ID NO: 419 and the sense strand comprises 18 consecutive nucleotides from 5′ end of SEQ ID NO: 204; or
wherein the antisense strand comprises 19 consecutive nucleotides from 5′ end of SEQ ID NO: 420 and the sense strand comprises 18 consecutive nucleotides from 5′ end of SEQ ID NO: 205.

4. The double-stranded polynucleic acid molecule of claim 1, wherein the polynucleic acid molecule comprises a 5′-terminal vinylphosphonate modified nucleotide.

5. The double-stranded polynucleic acid molecule of claim 4, wherein the 5′-terminal vinylphosphonate modified nucleotide is selected from: 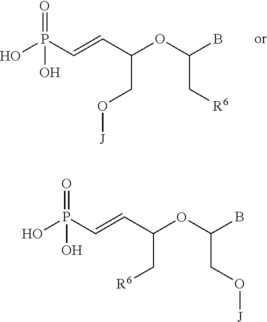
wherein B is a heterocyclic base moiety;
R⁶ is selected from hydrogen, halogen, alkyl or alkoxy; and
J is an internucleotide linking group linking to an adjacent nucleotide of the polynucleic acid molecule.

6. The double-stranded polynucleic acid molecule of claim 1, wherein each of the anti-sense strand or the sense strand is from 19 to 23 nucleotides in length.

7. A polynucleic acid molecule conjugate comprising:
an antibody or antigen binding fragment thereof conjugated to a polynucleic acid molecule that hybridizes to a target sequence of DUX4;
wherein the polynucleic acid molecule comprises 19 consecutive nucleotides from 5′ end of a nucleic acid sequence selected from SEQ ID NO: 412-420.

8. The polynucleic acid molecule conjugate of claim 7, wherein the antibody or antigen binding fragment thereof comprises a non-human antibody or antigen binding fragment thereof, a human antibody or antigen binding fragment thereof, a humanized antibody or antigen binding fragmet thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof.

9. The polynucleic acid molecule conjugate of claim 7, wherein the antibody or antigen binding fragment thereof is an anti-transferrin receptor antibody or antigen binding fragment thereof.

10. The polynucleic acid molecule conjugate of claim 7, wherein the polynucleic acid molecule conjugate comprises a linker connecting the antibody or antigen binding fragment thereof to the polynucleic acid molecule via a cysteine residue or a lysine residue on the antibody or antigen binding fragment thereof.

11. The polynucleic acid molecule conjugate of claim 10, wherein the linker is a C₁-C₆ alkyl linker; a homobifunctional linker or heterobifunctional linker, and comprises a maleimide group, a dipeptide moiety, a benzoic acid group, or its derivative thereof; or a cleavable or non-cleavable linker.

12. The polynucleic acid molecule conjugate of claim 7, wherein a ratio between the polynucleic acid molecule and the antibody or antigen binding fragment thereof is about 1:1, 2:1, 3:1, or 4:1.

13. A pharmaceutical composition comprising: a double-stranded polynucleic acid molecule of claim 1; and a pharmaceutically acceptable excipient.

14. A pharmaceutical composition comprising: a polynucleic acid molecule conjugate of claim 7; and a pharmaceutically acceptable excipient.

15. The pharmaceutical composition of claim 14, wherein the pharmaceutical composition is formulated as a nanoparticle formulation.

16. The pharmaceutical composition of claim 14, wherein the pharmaceutical composition is formulated for parenteral, oral, intranasal, buccal, rectal, transdermal, intravenous, subcutaneous, or intrathecal administration.

17. A method for modulating DUX4 expression in a subject, comprising:
providing a polynucleic acid conjugate comprising: an antibody or antigen binding fragment thereof conjugated to a polynucleic acid molecule that hybridizes to a target sequence of DUX4; wherein the polynucleic acid molecule comprises 19 consecutive nucleotides from 5′ end of a nucleic acid sequence selected from SEQ ID NO: 412-420; and
administering the polynucleic acid conjugate to the subject, thereby modulating DUX4 expression, wherein the polynucleic acid conjugate reduces a quantity of the mRNA transcript of DUX4 gene.

18. The method of claim 17, wherein the polynucleic acid molecule mediates RNA interference against the human DUX4 and modulates muscle atrophy in the subject.

19. The method of claim 17, wherein the RNA interference comprises reducing expression of mRNA transcript of DUX4 gene by at least 50%, at least 60%, or at least 70% or more compared to a quantity of the mRNA transcript of DUX4 gene in an untreated cell.

20. The method of claim 17, wherein the muscle atrophy is Facioscapulohumeral muscular dystrophy (FSHD).


SEQUENCE LISTING (438 sequences)

SEQ ID NO: 1 - DNA, 19 bp, synthetic construct
  source 1..19: mol_type = other DNA
cgacaccctcggacagcac

SEQ ID NO: 2 - DNA, 19 bp, synthetic construct
  source 1..19: mol_type = other DNA
acggcgacggagactcgtt

SEQ ID NO: 3 - DNA, 19 bp, synthetic construct
  source 1..19: mol_type = other DNA
cggcgacggagactcgttt

SEQ ID NO: 4 - DNA, 19 bp, synthetic construct
  source 1..19: mol_type = other DNA
ggcgacggagactcgtttg

SEQ ID NO: 5 - DNA, 19 bp, synthetic construct
  source 1..19: mol_type = other DNA
gcgacggagactcgtttgg

SEQ ID NO: 6 - DNA, 19 bp, synthetic construct
  source 1..19: mol_type = other DNA
cgacggagactcgtttgga

SEQ ID NO: 7 - DNA, 19 bp, synthetic construct
  source 1..19: mol_type = other DNA
gacggagactcgtttggac

SEQ ID NO: 8 - DNA, 19 bp, synthetic construct
  source 1..19: mol_type = other DNA
acggagactcgtttggacc

SEQ ID NO: 9 - DNA, 19 bp, synthetic construct
  source 1..19: mol_type = other DNA
ggaccccgagccaaagcga

SEQ ID NO: 10 - DNA, 19 bp, synthetic construct
  source 1..19: mol_type = other DNA
gaccccgagccaaagcgag

SEQ ID NO: 11 - DNA, 19 bp, synthetic construct
  source 1..19: mol_type = other DNA
accccgagccaaagcgagg

SEQ ID NO: 12 - DNA, 19 bp, synthetic construct
  source 1..19: mol_type = other DNA
cctgcgagcctgctttgag

SEQ ID NO: 13 - DNA, 19 bp, synthetic construct
  source 1..19: mol_type = other DNA
gcgagcctgctttgagcgg

SEQ ID NO: 14 - DNA, 19 bp, synthetic construct
  source 1..19: mol_type = other DNA
tcgccaccagagaacggct

SEQ ID NO: 15 - DNA, 19 bp, synthetic construct
  source 1..19: mol_type = other DNA
caggccatcggcattccgg

SEQ ID NO: 16 - DNA, 19 bp, synthetic construct
  source 1..19: mol_type = other DNA
ggccatcggcattccggag

SEQ ID NO: 17 - DNA, 19 bp, synthetic construct
  source 1..19: mol_type = other DNA
gccatcggcattccggagc

SEQ ID NO: 18 - DNA, 19 bp, synthetic construct
  source 1..19: mol_type = other DNA
gcaccggcgggaatctcgg

SEQ ID NO: 19 - DNA, 19 bp, synthetic construct
  source 1..19: mol_type = other DNA
caccggcgggaatctcggc

SEQ ID NO: 20 - DNA, 19 bp, synthetic construct
  source 1..19: mol_type = other DNA
ccagaaggccggcgaaagc

SEQ ID NO: 21 - DNA, 19 bp, synthetic construct
  source 1..19: mol_type = other DNA
agaaggccggcgaaagcgg

SEQ ID NO: 22 - DNA, 19 bp, synthetic construct
  source 1..19: mol_type = other DNA
gaaggccggcgaaagcgga

SEQ ID NO: 23 - DNA, 19 bp, synthetic construct
  source 1..19: mol_type = other DNA
gcgaaagcggaccgccgtc

SEQ ID NO: 24 - DNA, 19 bp, synthetic construct
  source 1..19: mol_type = other DNA
gaaagcggaccgccgtcac

SEQ ID NO: 25 - DNA, 19 bp, synthetic construct
  source 1..19: mol_type = other DNA
cggaccgccgtcaccggat

SEQ ID NO: 26 - DNA, 19 bp, synthetic construct
  source 1..19: mol_type = other DNA
ggaccgccgtcaccggatc

SEQ ID NO: 27 - DNA, 19 bp, synthetic construct
  source 1..19: mol_type = other DNA
gaccgccgtcaccggatcc

SEQ ID NO: 28 - DNA, 19 bp, synthetic construct
  source 1..19: mol_type = other DNA
agacgggcctcccggagtc

SEQ ID NO: 29 - DNA, 19 bp, synthetic construct
  source 1..19: mol_type = other DNA
cctcgtgggtcgccttcgc

SEQ ID NO: 30 - DNA, 19 bp, synthetic construct
  source 1..19: mol_type = other DNA
ctcgtgggtcgccttcgcc

SEQ ID NO: 31 - DNA, 19 bp, synthetic construct
  source 1..19: mol_type = other DNA
gaggggatctcccaacctg

SEQ ID NO: 32 - DNA, 19 bp, synthetic construct
  source 1..19: mol_type = other DNA
cgcgcggggatttcgccta

SEQ ID NO: 33 - DNA, 19 bp, synthetic construct
  source 1..19: mol_type = other DNA
gcgcggggatttcgcctac

SEQ ID NO: 34 - DNA, 19 bp, synthetic construct
  source 1..19: mol_type = other DNA
cggggatttcgcctacgcc

SEQ ID NO: 35 - DNA, 19 bp, synthetic construct
  source 1..19: mol_type = other DNA
tgcttgcgccacccacgtc

SEQ ID NO: 36 - DNA, 19 bp, synthetic construct
  source 1..19: mol_type = other DNA
ctggcgagcccggagtttc

SEQ ID NO: 37 - DNA, 19 bp, synthetic construct
  source 1..19: mol_type = other DNA
ggcgagcccggagtttctg

SEQ ID NO: 38 - DNA, 19 bp, synthetic construct
  source 1..19: mol_type = other DNA
ggcgcaacctctcctagaa

SEQ ID NO: 39 - DNA, 19 bp, synthetic construct
  source 1..19: mol_type = other DNA
gcgcaacctctcctagaaa

SEQ ID NO: 40 - DNA, 19 bp, synthetic construct
  source 1..19: mol_type = other DNA
aacctctcctagaaacgga

SEQ ID NO: 41 - DNA, 19 bp, synthetic construct
  source 1..19: mol_type = other DNA
cagcgaggaagaataccgg

SEQ ID NO: 42 - DNA, 19 bp, synthetic construct
  source 1..19: mol_type = other DNA
agcgaggaagaataccggg

SEQ ID NO: 43 - DNA, 19 bp, synthetic construct
  source 1..19: mol_type = other DNA
gcgaggaagaataccgggc

SEQ ID NO: 44 - DNA, 19 bp, synthetic construct
  source 1..19: mol_type = other DNA
gttgggacggggtcgggtg

SEQ ID NO: 45 - DNA, 19 bp, synthetic construct
  source 1..19: mol_type = other DNA
acggggtcgggtggttcgg

SEQ ID NO: 46 - DNA, 19 bp, synthetic construct
  source 1..19: mol_type = other DNA
ggtcgggtggttcggggca

SEQ ID NO: 47 - DNA, 19 bp, synthetic construct
  source 1..19: mol_type = other DNA
gtcgggtggttcggggcag

SEQ ID NO: 48 - DNA, 19 bp, synthetic construct
  source 1..19: mol_type = other DNA
gcggtggcctctctttcgc

SEQ ID NO: 49 - DNA, 19 bp, synthetic construct
  source 1..19: mol_type = other DNA
cggtggcctctctttcgcg

SEQ ID NO: 50 - DNA, 19 bp, synthetic construct
  source 1..19: mol_type = other DNA
ggtggcctctctttcgcgg

SEQ ID NO: 51 - DNA, 19 bp, synthetic construct
  source 1..19: mol_type = other DNA
gcctctctttcgcggggaa

SEQ ID NO: 52 - DNA, 19 bp, synthetic construct
  source 1..19: mol_type = other DNA
cacctggctggctacggag

SEQ ID NO: 53 - DNA, 19 bp, synthetic construct
  source 1..19: mol_type = other DNA
gctacggaggggcgtgtct

SEQ ID NO: 54 - DNA, 19 bp, synthetic construct
  source 1..19: mol_type = other DNA
ctacggaggggcgtgtctc

SEQ ID NO: 55 - DNA, 19 bp, synthetic construct
  source 1..19: mol_type = other DNA
acgtgcaagggagctcgct

SEQ ID NO: 56 - DNA, 19 bp, synthetic construct
  source 1..19: mol_type = other DNA
cgtgcaagggagctcgctg

SEQ ID NO: 57 - DNA, 19 bp, synthetic construct
  source 1..19: mol_type = other DNA
gtgcaagggagctcgctgg

SEQ ID NO: 58 - DNA, 19 bp, synthetic construct
  source 1..19: mol_type = other DNA
caccttccgacgctgtcta

SEQ ID NO: 59 - DNA, 19 bp, synthetic construct
  source 1..19: mol_type = other DNA
accttccgacgctgtctag

SEQ ID NO: 60 - DNA, 19 bp, synthetic construct
  source 1..19: mol_type = other DNA
ccttccgacgctgtctagg

SEQ ID NO: 61 - DNA, 19 bp, synthetic construct
  source 1..19: mol_type = other DNA
cttccgacgctgtctaggc

SEQ ID NO: 62 - DNA, 19 bp, synthetic construct
  source 1..19: mol_type = other DNA
tccgacgctgtctaggcaa

SEQ ID NO: 63 - DNA, 19 bp, synthetic construct
  source 1..19: mol_type = other DNA
ccgacgctgtctaggcaaa

SEQ ID NO: 64 - DNA, 19 bp, synthetic construct
  source 1..19: mol_type = other DNA
acgctgtctaggcaaacct

SEQ ID NO: 65 - DNA, 19 bp, synthetic construct
  source 1..19: mol_type = other DNA
aaacctggattagagttac

SEQ ID NO: 66 - DNA, 19 bp, synthetic construct
  source 1..19: mol_type = other DNA
ctttgagaaggatcgcttt

SEQ ID NO: 67 - DNA, 19 bp, synthetic construct
  source 1..19: mol_type = other DNA
gccggcagaggggatctcc

SEQ ID NO: 68 - DNA, 19 bp, synthetic construct
  source 1..19: mol_type = other DNA
gggccaaggggtgcttgcg

SEQ ID NO: 69 - DNA, 19 bp, synthetic construct
  source 1..19: mol_type = other DNA
gccaaggggtgcttgcgcc

SEQ ID NO: 70 - DNA, 19 bp, synthetic construct
  source 1..19: mol_type = other DNA
atgcaaggcatcccggcgc

SEQ ID NO: 71 - DNA, 19 bp, synthetic construct
  source 1..19: mol_type = other DNA
gtgctgtccgagggtgtcg

SEQ ID NO: 72 - DNA, 19 bp, synthetic construct
  source 1..19: mol_type = other DNA
aacgagtctccgtcgccgt

SEQ ID NO: 73 - DNA, 19 bp, synthetic construct
  source 1..19: mol_type = other DNA
aaacgagtctccgtcgccg

SEQ ID NO: 74 - DNA, 19 bp, synthetic construct
  source 1..19: mol_type = other DNA
caaacgagtctccgtcgcc

SEQ ID NO: 75 - DNA, 19 bp, synthetic construct
  source 1..19: mol_type = other DNA
ccaaacgagtctccgtcgc

SEQ ID NO: 76 - DNA, 19 bp, synthetic construct
  source 1..19: mol_type = other DNA
tccaaacgagtctccgtcg

SEQ ID NO: 77 - DNA, 19 bp, synthetic construct
  source 1..19: mol_type = other DNA
gtccaaacgagtctccgtc

SEQ ID NO: 78 - DNA, 19 bp, synthetic construct
  source 1..19: mol_type = other DNA
ggtccaaacgagtctccgt

SEQ ID NO: 79 - DNA, 19 bp, synthetic construct
  source 1..19: mol_type = other DNA
tcgctttggctcggggtcc

SEQ ID NO: 80 - DNA, 19 bp, synthetic construct
  source 1..19: mol_type = other DNA
ctcgctttggctcggggtc

SEQ ID NO: 81 - DNA, 19 bp, synthetic construct
  source 1..19: mol_type = other DNA
cctcgctttggctcggggt

SEQ ID NO: 82 - DNA, 19 bp, synthetic construct
  source 1..19: mol_type = other DNA
ctcaaagcaggctcgcagg

SEQ ID NO: 83 - DNA, 19 bp, synthetic construct
  source 1..19: mol_type = other DNA
ccgctcaaagcaggctcgc

SEQ ID NO: 84 - DNA, 19 bp, synthetic construct
  source 1..19: mol_type = other DNA
agccgttctctggtggcga

SEQ ID NO: 85 - DNA, 19 bp, synthetic construct
  source 1..19: mol_type = other DNA
ccggaatgccgatggcctg

SEQ ID NO: 86 - DNA, 19 bp, synthetic construct
  source 1..19: mol_type = other DNA
ctccggaatgccgatggcc

SEQ ID NO: 87 - DNA, 19 bp, synthetic construct
  source 1..19: mol_type = other DNA
gctccggaatgccgatggc

SEQ ID NO: 88 - DNA, 19 bp, synthetic construct
  source 1..19: mol_type = other DNA
ccgagattcccgccggtgc

SEQ ID NO: 89 - DNA, 19 bp, synthetic construct
  source 1..19: mol_type = other DNA
gccgagattcccgccggtg

SEQ ID NO: 90 - DNA, 19 bp, synthetic construct
  source 1..19: mol_type = other DNA
gctttcgccggccttctgg

SEQ ID NO: 91 - DNA, 19 bp, synthetic construct
  source 1..19: mol_type = other DNA
ccgctttcgccggccttct

SEQ ID NO: 92 - DNA, 19 bp, synthetic construct
  source 1..19: mol_type = other DNA
tccgctttcgccggccttc

SEQ ID NO: 93 - DNA, 19 bp, synthetic construct
  source 1..19: mol_type = other DNA
gacggcggtccgctttcgc

SEQ ID NO: 94 - DNA, 19 bp, synthetic construct
  source 1..19: mol_type = other DNA
gtgacggcggtccgctttc

SEQ ID NO: 95 - DNA, 19 bp, synthetic construct
  source 1..19: mol_type = other DNA
atccggtgacggcggtccg

SEQ ID NO: 96 - DNA, 19 bp, synthetic construct
  source 1..19: mol_type = other DNA
gatccggtgacggcggtcc

SEQ ID NO: 97 - DNA, 19 bp, synthetic construct
  source 1..19: mol_type = other DNA
ggatccggtgacggcggtc

SEQ ID NO: 98 - DNA, 19 bp, synthetic construct
  source 1..19: mol_type = other DNA
gactccgggaggcccgtct

SEQ ID NO: 99 - DNA, 19 bp, synthetic construct
  source 1..19: mol_type = other DNA
gcgaaggcgacccacgagg

SEQ ID NO: 100 - DNA, 19 bp, synthetic construct
  source 1..19: mol_type = other DNA
ggcgaaggcgacccacgag

SEQ ID NO: 101 - DNA, 19 bp, synthetic construct
  source 1..19: mol_type = other DNA
caggttgggagatcccctc

SEQ ID NO: 102 - DNA, 19 bp, synthetic construct
  source 1..19: mol_type = other DNA
taggcgaaatccccgcgcg

SEQ ID NO: 103 - DNA, 19 bp, synthetic construct
  source 1..19: mol_type = other DNA
gtaggcgaaatccccgcgc

SEQ ID NO: 104 - DNA, 19 bp, synthetic construct
  source 1..19: mol_type = other DNA
ggcgtaggcgaaatccccg

SEQ ID NO: 105 - DNA, 19 bp, synthetic construct
  source 1..19: mol_type = other DNA
gacgtgggtggcgcaagca

SEQ ID NO: 106 - DNA, 19 bp, synthetic construct
  source 1..19: mol_type = other DNA
gaaactccgggctcgccag

SEQ ID NO: 107 - DNA, 19 bp, synthetic construct
  source 1..19: mol_type = other DNA
cagaaactccgggctcgcc

SEQ ID NO: 108 - DNA, 19 bp, synthetic construct
  source 1..19: mol_type = other DNA
ttctaggagaggttgcgcc

SEQ ID NO: 109 - DNA, 19 bp, synthetic construct
  source 1..19: mol_type = other DNA
tttctaggagaggttgcgc

SEQ ID NO: 110 - DNA, 19 bp, synthetic construct
  source 1..19: mol_type = other DNA
tccgtttctaggagaggtt

SEQ ID NO: 111 - DNA, 19 bp, synthetic construct
  source 1..19: mol_type = other DNA
ccggtattcttcctcgctg

SEQ ID NO: 112 - DNA, 19 bp, synthetic construct
  source 1..19: mol_type = other DNA
cccggtattcttcctcgct

SEQ ID NO: 113 - DNA, 19 bp, synthetic construct
  source 1..19: mol_type = other DNA
gcccggtattcttcctcgc

SEQ ID NO: 114 - DNA, 19 bp, synthetic construct
  source 1..19: mol_type = other DNA
cacccgaccccgtcccaac

SEQ ID NO: 115 - DNA, 19 bp, synthetic construct
  source 1..19: mol_type = other DNA
ccgaaccacccgaccccgt

SEQ ID NO: 116 - DNA, 19 bp, synthetic construct
  source 1..19: mol_type = other DNA
tgccccgaaccacccgacc

SEQ ID NO: 117 - DNA, 19 bp, synthetic construct
  source 1..19: mol_type = other DNA
ctgccccgaaccacccgac

SEQ ID NO: 118 - DNA, 19 bp, synthetic construct
  source 1..19: mol_type = other DNA
gcgaaagagaggccaccgc

SEQ ID NO: 119 - DNA, 19 bp, synthetic construct
  source 1..19: mol_type = other DNA
cgcgaaagagaggccaccg

SEQ ID NO: 120 - DNA, 19 bp, synthetic construct
  source 1..19: mol_type = other DNA
ccgcgaaagagaggccacc

SEQ ID NO: 121 - DNA, 19 bp, synthetic construct
  source 1..19: mol_type = other DNA
ttccccgcgaaagagaggc

SEQ ID NO: 122 - DNA, 19 bp, synthetic construct
  source 1..19: mol_type = other DNA
ctccgtagccagccaggtg

SEQ ID NO: 123 - DNA, 19 bp, synthetic construct
  source 1..19: mol_type = other DNA
agacacgcccctccgtagc

SEQ ID NO: 124 - DNA, 19 bp, synthetic construct
  source 1..19: mol_type = other DNA
gagacacgcccctccgtag

SEQ ID NO: 125 - DNA, 19 bp, synthetic construct
  source 1..19: mol_type = other DNA
agcgagctcccttgcacgt

SEQ ID NO: 126 - DNA, 19 bp, synthetic construct
  source 1..19: mol_type = other DNA
cagcgagctcccttgcacg

SEQ ID NO: 127 - DNA, 19 bp, synthetic construct
  source 1..19: mol_type = other DNA
ccagcgagctcccttgcac

SEQ ID NO: 128 - DNA, 19 bp, synthetic construct
  source 1..19: mol_type = other DNA
tagacagcgtcggaaggtg

SEQ ID NO: 129 - DNA, 19 bp, synthetic construct
  source 1..19: mol_type = other DNA
ctagacagcgtcggaaggt

SEQ ID NO: 130 - DNA, 19 bp, synthetic construct
  source 1..19: mol_type = other DNA
cctagacagcgtcggaagg

SEQ ID NO: 131 - DNA, 19 bp, synthetic construct
  source 1..19: mol_type = other DNA
gcctagacagcgtcggaag

SEQ ID NO: 132 - DNA, 19 bp, synthetic construct
  source 1..19: mol_type = other DNA
ttgcctagacagcgtcgga

SEQ ID NO: 133 - DNA, 19 bp, synthetic construct
  source 1..19: mol_type = other DNA
tttgcctagacagcgtcgg

SEQ ID NO: 134 - DNA, 19 bp, synthetic construct
  source 1..19: mol_type = other DNA
aggtttgcctagacagcgt

SEQ ID NO: 135 - DNA, 19 bp, synthetic construct
  source 1..19: mol_type = other DNA
gtaactctaatccaggttt

SEQ ID NO: 136 - DNA, 19 bp, synthetic construct
  source 1..19: mol_type = other DNA
aaagcgatccttctcaaag

SEQ ID NO: 137 - DNA, 19 bp, synthetic construct
  source 1..19: mol_type = other DNA
ggagatcccctctgccggc

SEQ ID NO: 138 - DNA, 19 bp, synthetic construct
  source 1..19: mol_type = other DNA
cgcaagcaccccttggccc

SEQ ID NO: 139 - DNA, 19 bp, synthetic construct
  source 1..19: mol_type = other DNA
ggcgcaagcaccccttggc

SEQ ID NO: 140 - DNA, 19 bp, synthetic construct
  source 1..19: mol_type = other DNA
gcgccgggatgccttgcat

SEQ ID NO: 141 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methylcytidine
  modified_base 15: mod_base = OTHER; note = 2'-O-methyladenosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methyladenosine phosphorothioate
  modified_base 19: mod_base = OTHER; note = 2'-O-methyladenosine
cgacaccctcggacagcaa

SEQ ID NO: 142 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methyluridine phosphorothioate
  modified_base 19: mod_base = OTHER; note = 2'-O-methyladenosine
acggcgacggagactcgta

SEQ ID NO: 143 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methyluridine phosphorothioate
  modified_base 18: mod_base = OTHER; note = 2'-O-methyluridine phosphorothioate
  modified_base 19: mod_base = OTHER; note = 2'-O-methyladenosine
cggcgacggagactcgtta

SEQ ID NO: 144 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methyluridine phosphorothioate
  modified_base 18: mod_base = OTHER; note = 2'-O-methyluridine phosphorothioate
  modified_base 19: mod_base = OTHER; note = 2'-O-methyladenosine
ggcgacggagactcgttta

SEQ ID NO: 145 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methyluridine phosphorothioate
  modified_base 18: mod_base = OTHER; note = 2'-O-methylguanosine phosphorothioate
  modified_base 19: mod_base = OTHER; note = 2'-O-methyladenosine
gcgacggagactcgtttga

SEQ ID NO: 146 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
cgacggagactcgtttgga

SEQ ID NO: 147 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
gacggagactcgtttggaa

SEQ ID NO: 148 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methyluridine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methyladenosine phosphorothioate
  modified_base 18: mod_base = OTHER; note = 2'-O-methylcytidine phosphorothioate
  modified_base 19: mod_base = OTHER; note = 2'-O-methyladenosine
acggagactcgtttggaca

SEQ ID NO: 149 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
ggaccccgagccaaagcga

SEQ ID NO: 150 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroguanosine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
gaccccgagccaaagcgaa

SEQ ID NO: 151 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methylguanosine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methyladenosine phosphorothioate
  modified_base 18: mod_base = OTHER; note = 2'-O-methylguanosine phosphorothioate
  modified_base 19: mod_base = OTHER; note = 2'-O-methyladenosine
accccgagccaaagcgaga

SEQ ID NO: 152 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylcytidine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
cctgcgagcctgctttgaa

SEQ ID NO: 153 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluorouridine
  modified_base 9: mod_base = OTHER; note = 2'-fluoroguanosine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methylguanosine
  modified_base 15: mod_base = OTHER; note = 2'-O-methyladenosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
gcgagcctgctttgagcga

SEQ ID NO: 154 - RNA, 19 nt, synthetic construct
  source 1..19: mol_type = other RNA
  modified_base 1: mod_base = OTHER; note = 2'-O-methyluridine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluoroadenosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
tcgccaccagagaacggca

SEQ ID NO: 155 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyladenosine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methylcytidine
  modified_base 7: mod_base = OTHER; note = 2'-fluoroadenosine
  modified_base 8: mod_base = OTHER; note = 2'-fluorouridine
  modified_base 9: mod_base = OTHER; note = 2'-fluorocytidine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyladenosine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
caggccatcggcattccga

SEQ ID NO: 156 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-O-methyluridine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
ggccatcggcattccggaa

SEQ ID NO: 157 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyluridine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methyladenosine phosphorothioate
  modified_base 18: mod_base = OTHER; note = 2'-O-methylguanosine phosphorothioate
  modified_base 19: mod_base = OTHER; note = 2'-O-methyladenosine
gccatcggcattccggaga

SEQ ID NO: 158 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methyluridine
  modified_base 15: mod_base = OTHER; note = 2'-O-methylcytidine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
gcaccggcgggaatctcga

SEQ ID NO: 159 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methyladenosine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
caccggcgggaatctcgga

SEQ ID NO: 160 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylcytidine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine phosphorothioate
  modified_base 18: mod_base = OTHER; note = 2'-O-methylguanosine phosphorothioate
  modified_base 19: mod_base = OTHER; note = 2'-O-methyladenosine
ccagaaggccggcgaaaga

SEQ ID NO: 161 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylguanosine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
agaaggccggcgaaagcga

SEQ ID NO: 162 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyladenosine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
gaaggccggcgaaagcgga

SEQ ID NO: 163 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methylcytidine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methyluridine phosphorothioate
  modified_base 19: mod_base = OTHER; note = 2'-O-methyladenosine
gcgaaagcggaccgccgta

SEQ ID NO: 164 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyladenosine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methyladenosine phosphorothioate
  modified_base 19: mod_base = OTHER; note = 2'-O-methyladenosine
gaaagcggaccgccgtcaa

SEQ ID NO: 165 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluorocytidine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyladenosine
  modified_base 14: mod_base = OTHER; note = 2'-O-methylcytidine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
cggaccgccgtcaccggaa

SEQ ID NO: 166 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methyladenosine phosphorothioate
  modified_base 18: mod_base = OTHER; note = 2'-O-methyluridine phosphorothioate
  modified_base 19: mod_base = OTHER; note = 2'-O-methyladenosine
ggaccgccgtcaccggata

SEQ ID NO: 167 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methylguanosine
  modified_base 16: mod_base = OTHER; note = 2'-O-methyladenosine
  modified_base 17: mod_base = OTHER; note = 2'-O-methyluridine phosphorothioate
  modified_base 18: mod_base = OTHER; note = 2'-O-methylcytidine phosphorothioate
  modified_base 19: mod_base = OTHER; note = 2'-O-methyladenosine
gaccgccgtcaccggatca

SEQ ID NO: 168 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylguanosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methylguanosine
  modified_base 16: mod_base = OTHER; note = 2'-O-methyladenosine
  modified_base 17: mod_base = OTHER; note = 2'-O-methylguanosine phosphorothioate
  modified_base 18: mod_base = OTHER; note = 2'-O-methyluridine phosphorothioate
  modified_base 19: mod_base = OTHER; note = 2'-O-methyladenosine
agacgggcctcccggagta

SEQ ID NO: 169 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylcytidine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methyluridine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
cctcgtgggtcgccttcga

SEQ ID NO: 170 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyluridine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
ctcgtgggtcgccttcgca

SEQ ID NO: 171 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyladenosine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluorouridine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methylcytidine
  modified_base 17: mod_base = OTHER; note = 2'-O-methylcytidine phosphorothioate
  modified_base 18: mod_base = OTHER; note = 2'-O-methyluridine phosphorothioate
  modified_base 19: mod_base = OTHER; note = 2'-O-methyladenosine
gaggggatctcccaaccta

SEQ ID NO: 172 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methylcytidine phosphorothioate
  modified_base 18: mod_base = OTHER; note = 2'-O-methyluridine phosphorothioate
  modified_base 19: mod_base = OTHER; note = 2'-O-methyladenosine
cgcgcggggatttcgccta

SEQ ID NO: 173 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methyluridine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methylcytidine
  modified_base 16: mod_base = OTHER; note = 2'-O-methylcytidine
  modified_base 17: mod_base = OTHER; note = 2'-O-methyluridine phosphorothioate
  modified_base 18: mod_base = OTHER; note = 2'-O-methyladenosine phosphorothioate
  modified_base 19: mod_base = OTHER; note = 2'-O-methyladenosine
gcgcggggatttcgcctaa

SEQ ID NO: 174 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methyladenosine
  modified_base 7: mod_base = OTHER; note = 2'-fluorouridine
  modified_base 8: mod_base = OTHER; note = 2'-fluorouridine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methyladenosine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
cggggatttcgcctacgca

SEQ ID NO: 175 - RNA, 19 nt, synthetic construct
  source 1..19: mol_type = other RNA
  modified_base 1: mod_base = OTHER; note = 2'-O-methyluridine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methyladenosine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methyluridine phosphorothioate
  modified_base 19: mod_base = OTHER; note = 2'-O-methyladenosine
tgcttgcgccacccacgta

SEQ ID NO: 176 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyluridine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-O-methyladenosine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methyluridine phosphorothioate
  modified_base 18: mod_base = OTHER; note = 2'-O-methyluridine phosphorothioate
  modified_base 19: mod_base = OTHER; note = 2'-O-methyladenosine
ctggcgagcccggagttta

SEQ ID NO: 177 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluorocytidine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methyluridine phosphorothioate
  modified_base 19: mod_base = OTHER; note = 2'-O-methyladenosine
ggcgagcccggagtttcta

SEQ ID NO: 178 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methyladenosine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
ggcgcaacctctcctagaa

SEQ ID NO: 179 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methyladenosine
  modified_base 16: mod_base = OTHER; note = 2'-O-methylguanosine
  modified_base 17: mod_base = OTHER; note = 2'-O-methyladenosine phosphorothioate
  modified_base 18: mod_base = OTHER; note = 2'-O-methyladenosine phosphorothioate
  modified_base 19: mod_base = OTHER; note = 2'-O-methyladenosine
gcgcaacctctcctagaaa

SEQ ID NO: 180 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-O-methylcytidine
  modified_base 7: mod_base = OTHER; note = 2'-fluorouridine
  modified_base 8: mod_base = OTHER; note = 2'-fluorocytidine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
aacctctcctagaaacgga

SEQ ID NO: 181 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyladenosine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methyluridine
  modified_base 15: mod_base = OTHER; note = 2'-O-methyladenosine
  modified_base 16: mod_base = OTHER; note = 2'-O-methylcytidine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
cagcgaggaagaataccga

SEQ ID NO: 182 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroadenosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methyladenosine
  modified_base 13: mod_base = OTHER; note = 2'-O-methyluridine
  modified_base 14: mod_base = OTHER; note = 2'-O-methyladenosine
  modified_base 15: mod_base = OTHER; note = 2'-O-methylcytidine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
agcgaggaagaataccgga

SEQ ID NO: 183 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluoroadenosine
  modified_base 9: mod_base = OTHER; note = 2'-fluoroguanosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-O-methyluridine
  modified_base 13: mod_base = OTHER; note = 2'-O-methyladenosine
  modified_base 14: mod_base = OTHER; note = 2'-O-methylcytidine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
gcgaggaagaataccggga

SEQ ID NO: 184 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyluridine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methyluridine phosphorothioate
  modified_base 19: mod_base = OTHER; note = 2'-O-methyladenosine
gttgggacggggtcgggta

SEQ ID NO: 185 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluorouridine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methyluridine
  modified_base 13: mod_base = OTHER; note = 2'-O-methylguanosine
  modified_base 14: mod_base = OTHER; note = 2'-O-methylguanosine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
acggggtcgggtggttcga

SEQ ID NO: 186 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluorouridine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
ggtcgggtggttcggggca

SEQ ID NO: 187 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyluridine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-O-methylguanosine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methyladenosine phosphorothioate
  modified_base 19: mod_base = OTHER; note = 2'-O-methyladenosine
gtcgggtggttcggggcaa

SEQ ID NO: 188 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
gcggtggcctctctttcga

SEQ ID NO: 189 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
cggtggcctctctttcgca

SEQ ID NO: 190 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluorouridine
  modified_base 9: mod_base = OTHER; note = 2'-fluorocytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methyluridine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
ggtggcctctctttcgcga

SEQ ID NO: 191 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methylcytidine
  modified_base 6: mod_base = OTHER; note = 2'-O-methyluridine
  modified_base 7: mod_base = OTHER; note = 2'-fluorocytidine
  modified_base 8: mod_base = OTHER; note = 2'-fluorouridine
  modified_base 9: mod_base = OTHER; note = 2'-fluorouridine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
gcctctctttcgcggggaa

SEQ ID NO: 192 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methyladenosine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylguanosine phosphorothioate
  modified_base 18: mod_base = OTHER; note = 2'-O-methyladenosine phosphorothioate
  modified_base 19: mod_base = OTHER; note = 2'-O-methyladenosine
cacctggctggctacggaa

SEQ ID NO: 193 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methyluridine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methylguanosine
  modified_base 15: mod_base = OTHER; note = 2'-O-methyluridine
  modified_base 16: mod_base = OTHER; note = 2'-O-methylguanosine
  modified_base 17: mod_base = OTHER; note = 2'-O-methyluridine phosphorothioate
  modified_base 18: mod_base = OTHER; note = 2'-O-methylcytidine phosphorothioate
  modified_base 19: mod_base = OTHER; note = 2'-O-methyladenosine
gctacggaggggcgtgtca

SEQ ID NO: 194 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyluridine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-O-methyluridine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methyluridine phosphorothioate
  modified_base 19: mod_base = OTHER; note = 2'-O-methyladenosine
ctacggaggggcgtgtcta

SEQ ID NO: 195 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluoroadenosine
  modified_base 8: mod_base = OTHER; note = 2'-fluoroadenosine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
acgtgcaagggagctcgca

SEQ ID NO: 196 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylguanosine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methylcytidine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methyluridine phosphorothioate
  modified_base 19: mod_base = OTHER; note = 2'-O-methyladenosine
cgtgcaagggagctcgcta

SEQ ID NO: 197 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methyluridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methyluridine phosphorothioate
  modified_base 18: mod_base = OTHER; note = 2'-O-methylguanosine phosphorothioate
  modified_base 19: mod_base = OTHER; note = 2'-O-methyladenosine
gtgcaagggagctcgctga

SEQ ID NO: 198 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyladenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-O-methyluridine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methyluridine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methylcytidine phosphorothioate
  modified_base 18: mod_base = OTHER; note = 2'-O-methyluridine phosphorothioate
  modified_base 19: mod_base = OTHER; note = 2'-O-methyladenosine
caccttccgacgctgtcta

SEQ ID NO: 199 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyluridine
  modified_base 6: mod_base = OTHER; note = 2'-O-methylcytidine
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methylguanosine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methyluridine phosphorothioate
  modified_base 18: mod_base = OTHER; note = 2'-O-methyladenosine phosphorothioate
  modified_base 19: mod_base = OTHER; note = 2'-O-methyladenosine
accttccgacgctgtctaa

SEQ ID NO: 200 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylcytidine phosphorothioate
  modified_base 3: mod_base = OTHER; note = 2'-O-methyluridine
  modified_base 4: mod_base = OTHER; note = 2'-O-methyluridine
  modified_base 5: mod_base = OTHER; note = 2'-O-methylcytidine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluorocytidine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylguanosine
  modified_base 14: mod_base = OTHER; note = 2'-O-methyluridine
  modified_base 15: mod_base = OTHER; note = 2'-O-methylcytidine
  modified_base 16: mod_base = OTHER; note = 2'-O-methyluridine
  modified_base 17: mod_base = OTHER; note = 2'-O-methyladenosine phosphorothioate
  modified_base 18: mod_base = OTHER; note = 2'-O-methylguanosine phosphorothioate
  modified_base 19: mod_base = OTHER; note = 2'-O-methyladenosine
ccttccgacgctgtctaga

SEQ ID NO: 201 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyluridine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluoroguanosine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methyladenosine
  modified_base 17: mod_base = OTHER; note = 2'-O-methylguanosine phosphorothioate
  modified_base 18: mod_base = OTHER; note = 2'-O-methylguanosine phosphorothioate
  modified_base 19: mod_base = OTHER; note = 2'-O-methyladenosine
cttccgacgctgtctagga

SEQ ID NO: 202 - RNA, 19 nt, synthetic construct
  source 1..19: mol_type = other RNA
  modified_base 1: mod_base = OTHER; note = 2'-O-methyluridine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-O-methylcytidine
  modified_base 7: mod_base = OTHER; note = 2'-fluoroguanosine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methyladenosine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methyladenosine phosphorothioate
  modified_base 19: mod_base = OTHER; note = 2'-O-methyladenosine
tccgacgctgtctaggcaa

SEQ ID NO: 203 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-O-methylguanosine
  modified_base 7: mod_base = OTHER; note = 2'-fluorocytidine
  modified_base 8: mod_base = OTHER; note = 2'-fluorouridine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methyladenosine
  modified_base 14: mod_base = OTHER; note = 2'-O-methylguanosine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methyladenosine phosphorothioate
  modified_base 18: mod_base = OTHER; note = 2'-O-methyladenosine phosphorothioate
  modified_base 19: mod_base = OTHER; note = 2'-O-methyladenosine
ccgacgctgtctaggcaaa

SEQ ID NO: 204 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methylcytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-O-methylguanosine
  modified_base 7: mod_base = OTHER; note = 2'-fluorouridine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-O-methyladenosine
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methylcytidine phosphorothioate
  modified_base 18: mod_base = OTHER; note = 2'-O-methylcytidine phosphorothioate
  modified_base 19: mod_base = OTHER; note = 2'-O-methyladenosine
acgctgtctaggcaaacca

SEQ ID NO: 205 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methyladenosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methyluridine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methyluridine
  modified_base 11: mod_base = OTHER; note = 2'-O-methyluridine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-O-methyladenosine
  modified_base 15: mod_base = OTHER; note = 2'-O-methylguanosine
  modified_base 16: mod_base = OTHER; note = 2'-O-methyluridine
  modified_base 17: mod_base = OTHER; note = 2'-O-methyluridine phosphorothioate
  modified_base 18: mod_base = OTHER; note = 2'-O-methyladenosine phosphorothioate
  modified_base 19: mod_base = OTHER; note = 2'-O-methyladenosine
aaacctggattagagttaa

SEQ ID NO: 206 - RNA, 19 nt, synthetic construct
  source 1..19: mol_type = other RNA
  modified_base 1: mod_base = OTHER; note = 2'-O-methylcytidine phosphorothioate
  modified_base 2: mod_base = OTHER; note = 2'-O-methyluridine phosphorothioate
  modified_base 3: mod_base = OTHER; note = 2'-O-methyluridine
  modified_base 4: mod_base = OTHER; note = 2'-O-methyluridine
  modified_base 5: mod_base = OTHER; note = 2'-O-methylguanosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroadenosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyluridine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methyluridine phosphorothioate
  modified_base 18: mod_base = OTHER; note = 2'-O-methyluridine phosphorothioate
  modified_base 19: mod_base = OTHER; note = 2'-O-methyladenosine
ctttgagaaggatcgctta

SEQ ID NO: 207 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-O-methylcytidine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-O-methyladenosine
  modified_base 15: mod_base = OTHER; note = 2'-O-methyluridine
  modified_base 16: mod_base = OTHER; note = 2'-O-methylcytidine
  modified_base 17: mod_base = OTHER; note = 2'-O-methyluridine phosphorothioate
  modified_base 18: mod_base = OTHER; note = 2'-O-methylcytidine phosphorothioate
  modified_base 19: mod_base = OTHER; note = 2'-O-methyladenosine
gccggcagaggggatctca

SEQ ID NO: 208 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylguanosine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methyluridine
  modified_base 13: mod_base = OTHER; note = 2'-O-methylguanosine
  modified_base 14: mod_base = OTHER; note = 2'-O-methylcytidine
  modified_base 15: mod_base = OTHER; note = 2'-O-methyluridine
  modified_base 16: mod_base = OTHER; note = 2'-O-methyluridine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
gggccaaggggtgcttgca

SEQ ID NO: 209 - RNA, 19 nt, synthetic construct
  source 1..19: mol_type = other RNA
  modified_base 1: mod_base = OTHER; note = 2'-O-methylguanosine phosphorothioate
  modified_base 2: mod_base = OTHER; note = 2'-O-methylcytidine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-O-methyluridine
  modified_base 15: mod_base = OTHER; note = 2'-O-methylguanosine
  modified_base 16: mod_base = OTHER; note = 2'-O-methylcytidine
  modified_base 17: mod_base = OTHER; note = 2'-O-methylguanosine phosphorothioate
  modified_base 18: mod_base = OTHER; note = 2'-O-methylcytidine phosphorothioate
  modified_base 19: mod_base = OTHER; note = 2'-O-methyladenosine
gccaaggggtgcttgcgca

SEQ ID NO: 210 - RNA, 19 nt, synthetic construct
  source 1..19: mol_type = other RNA
  modified_base 1: mod_base = OTHER; note = 2'-O-methyladenosine phosphorothioate
  modified_base 2: mod_base = OTHER; note = 2'-O-methyluridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-O-methylcytidine
  modified_base 15: mod_base = OTHER; note = 2'-O-methylguanosine
  modified_base 16: mod_base = OTHER; note = 2'-O-methylguanosine
  modified_base 17: mod_base = OTHER; note = 2'-O-methylcytidine phosphorothioate
  modified_base 18: mod_base = OTHER; note = 2'-O-methylguanosine phosphorothioate
  modified_base 19: mod_base = OTHER; note = 2'-O-methyladenosine
atgcaaggcatcccggcga

SEQ ID NO: 211 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methyluridine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
ttgctgtccgagggtgtcgtt

SEQ ID NO: 212 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tacgagtctccgtcgccgttt

SEQ ID NO: 213 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
taacgagtctccgtcgccgtt

SEQ ID NO: 214 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
taaacgagtctccgtcgcctt

SEQ ID NO: 215 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcaaacgagtctccgtcgctt

SEQ ID NO: 216 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylcytidine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tccaaacgagtctccgtcgtt

SEQ ID NO: 217 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttccaaacgagtctccgtctt

SEQ ID NO: 218 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tgtccaaacgagtctccgttt

SEQ ID NO: 219 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methyluridine
  modified_base 8: mod_base = OTHER; note = 2'-O-methylguanosine
  modified_base 9: mod_base = OTHER; note = 2'-O-methylguanosine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcgctttggctcggggtcctt

SEQ ID NO: 220 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyluridine
  modified_base 8: mod_base = OTHER; note = 2'-O-methyluridine
  modified_base 9: mod_base = OTHER; note = 2'-O-methylguanosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttcgctttggctcggggtctt

SEQ ID NO: 221 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluorocytidine
  modified_base 7: mod_base = OTHER; note = 2'-O-methyluridine
  modified_base 8: mod_base = OTHER; note = 2'-O-methyluridine
  modified_base 9: mod_base = OTHER; note = 2'-O-methyluridine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tctcgctttggctcggggttt

SEQ ID NO: 222 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
ttcaaagcaggctcgcaggtt

SEQ ID NO: 223 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcgctcaaagcaggctcgctt

SEQ ID NO: 224 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluorouridine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyladenosine phosphorothioate
  modified_base 20: mod_base = OTHER; note = 2'-O-methyluridine phosphorothioate
  modified_base 21: mod_base = OTHER; note = 2'-O-methyluridine
tgccgttctctggtggcgatt

SEQ ID NO: 225 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyluridine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyluridine
  modified_base 14: mod_base = OTHER; note = 2'-fluoroguanosine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methyluridine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tcggaatgccgatggcctgtt

SEQ ID NO: 226 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyluridine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluoroadenosine
  modified_base 15: mod_base = OTHER; note = 2'-O-methyluridine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttccggaatgccgatggcctt

SEQ ID NO: 227 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyluridine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluorouridine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tctccggaatgccgatggctt

SEQ ID NO: 228 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methyluridine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcgagattcccgccggtgctt

SEQ ID NO: 229 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyluridine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tccgagattcccgccggtgtt

SEQ ID NO: 230 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methyluridine
  modified_base 4: mod_base = OTHER; note = 2'-O-methyluridine
  modified_base 5: mod_base = OTHER; note = 2'-O-methyluridine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methyluridine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tctttcgccggccttctggtt

SEQ ID NO: 231 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluorouridine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tcgctttcgccggccttcttt

SEQ ID NO: 232 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyluridine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tccgctttcgccggccttctt

SEQ ID NO: 233 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tacggcggtccgctttcgctt

SEQ ID NO: 234 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluorouridine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttgacggcggtccgctttctt

SEQ ID NO: 235 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
ttccggtgacggcggtccgtt

SEQ ID NO: 236 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methyluridine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tatccggtgacggcggtcctt

SEQ ID NO: 237 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methyladenosine
  modified_base 4: mod_base = OTHER; note = 2'-O-methyluridine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methyluridine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tgatccggtgacggcggtctt

SEQ ID NO: 238 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tactccgggaggcccgtcttt

SEQ ID NO: 239 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroadenosine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tcgaaggcgacccacgaggtt

SEQ ID NO: 240 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tgcgaaggcgacccacgagtt

SEQ ID NO: 241 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methyluridine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
taggttgggagatcccctctt

SEQ ID NO: 242 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
taggcgaaatccccgcgcgtt

SEQ ID NO: 243 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttaggcgaaatccccgcgctt

SEQ ID NO: 244 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tgcgtaggcgaaatccccgtt

SEQ ID NO: 245 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methyluridine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methyladenosine phosphorothioate
  modified_base 20: mod_base = OTHER; note = 2'-O-methyluridine phosphorothioate
  modified_base 21: mod_base = OTHER; note = 2'-O-methyluridine
tacgtgggtggcgcaagcatt

SEQ ID NO: 246 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methylcytidine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methylguanosine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
taaactccgggctcgccagtt

SEQ ID NO: 247 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylcytidine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tagaaactccgggctcgcctt

SEQ ID NO: 248 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttctaggagaggttgcgcctt

SEQ ID NO: 249 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methyluridine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tttctaggagaggttgcgctt

SEQ ID NO: 250 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tccgtttctaggagaggtttt

SEQ ID NO: 251 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylguanosine
  modified_base 5: mod_base = OTHER; note = 2'-O-methyluridine
  modified_base 6: mod_base = OTHER; note = 2'-fluoroadenosine
  modified_base 7: mod_base = OTHER; note = 2'-O-methyluridine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methyluridine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methyluridine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tcggtattcttcctcgctgtt

SEQ ID NO: 252 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylguanosine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methyluridine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylcytidine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tccggtattcttcctcgcttt

SEQ ID NO: 253 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-O-methyladenosine
  modified_base 9: mod_base = OTHER; note = 2'-O-methyluridine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcccggtattcttcctcgctt

SEQ ID NO: 254 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tacccgaccccgtcccaactt

SEQ ID NO: 255 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methylcytidine
  modified_base 8: mod_base = OTHER; note = 2'-O-methyladenosine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tcgaaccacccgaccccgttt

SEQ ID NO: 256 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tgccccgaaccacccgacctt

SEQ ID NO: 257 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methylcytidine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methyladenosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttgccccgaaccacccgactt

SEQ ID NO: 258 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcgaaagagaggccaccgctt

SEQ ID NO: 259 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroadenosine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tgcgaaagagaggccaccgtt

SEQ ID NO: 260 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcgcgaaagagaggccacctt

SEQ ID NO: 261 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluoroadenosine
  modified_base 15: mod_base = OTHER; note = 2'-O-methylguanosine
  modified_base 16: mod_base = OTHER; note = 2'-fluoroadenosine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
ttccccgcgaaagagaggctt

SEQ ID NO: 262 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylcytidine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
ttccgtagccagccaggtgtt

SEQ ID NO: 263 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methyladenosine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tgacacgcccctccgtagctt

SEQ ID NO: 264 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluoroadenosine
  modified_base 7: mod_base = OTHER; note = 2'-O-methylcytidine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tagacacgcccctccgtagtt

SEQ ID NO: 265 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methylcytidine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluoroadenosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tgcgagctcccttgcacgttt

SEQ ID NO: 266 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tagcgagctcccttgcacgtt

SEQ ID NO: 267 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methyladenosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tcagcgagctcccttgcactt

SEQ ID NO: 268 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylguanosine
  modified_base 10: mod_base = OTHER; note = 2'-O-methyluridine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methylguanosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tagacagcgtcggaaggtgtt

SEQ ID NO: 269 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylguanosine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroadenosine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
ttagacagcgtcggaaggttt

SEQ ID NO: 270 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methyluridine
  modified_base 4: mod_base = OTHER; note = 2'-O-methyladenosine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methylcytidine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methylcytidine
  modified_base 11: mod_base = OTHER; note = 2'-O-methylguanosine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methylguanosine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tctagacagcgtcggaaggtt

SEQ ID NO: 271 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methylcytidine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tcctagacagcgtcggaagtt

SEQ ID NO: 272 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methyladenosine phosphorothioate
  modified_base 20: mod_base = OTHER; note = 2'-O-methyluridine phosphorothioate
  modified_base 21: mod_base = OTHER; note = 2'-O-methyluridine
ttgcctagacagcgtcggatt

SEQ ID NO: 273 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methyluridine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tttgcctagacagcgtcggtt

SEQ ID NO: 274 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methyluridine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tggtttgcctagacagcgttt

SEQ ID NO: 275 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorouridine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methylcytidine
  modified_base 8: mod_base = OTHER; note = 2'-O-methyluridine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroadenosine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
ttaactctaatccaggttttt

SEQ ID NO: 276 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroadenosine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
taagcgatccttctcaaagtt

SEQ ID NO: 277 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluorouridine
  modified_base 7: mod_base = OTHER; note = 2'-O-methylcytidine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluoroguanosine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tgagatcccctctgccggctt

SEQ ID NO: 278 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methylcytidine
  modified_base 8: mod_base = OTHER; note = 2'-O-methyladenosine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylcytidine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tgcaagcaccccttggccctt

SEQ ID NO: 279 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluoroguanosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methylcytidine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorocytidine
  modified_base 15: mod_base = OTHER; note = 2'-O-methyluridine
  modified_base 16: mod_base = OTHER; note = 2'-fluorouridine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methylcytidine phosphorothioate
  modified_base 20: mod_base = OTHER; note = 2'-O-methyluridine phosphorothioate
  modified_base 21: mod_base = OTHER; note = 2'-O-methyluridine
tgcgcaagcaccccttggctt

SEQ ID NO: 280 - RNA, 21 nt, synthetic construct
  source 1..21: mol_type = other RNA
  modified_base 1: mod_base = OTHER; note = 2'-fluorouridine phosphorothioate
  modified_base 2: mod_base = OTHER; note = 2'-fluorocytidine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methylguanosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methyluridine
  modified_base 11: mod_base = OTHER; note = 2'-O-methylguanosine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methyladenosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tcgccgggatgccttgcattt

SEQ ID NO: 281 - protein, 9 aa, synthetic construct
YTFTNYWMH

SEQ ID NO: 282 - protein, 17 aa, synthetic construct
EINPINGRSNYAQKFQG

SEQ ID NO: 283 - protein, 7 aa, synthetic construct
GTRAMHY

SEQ ID NO: 284 - protein, 17 aa, synthetic construct
EINPINGRSNYAEKFQG

SEQ ID NO: 285 - protein, 17 aa, synthetic construct
EINPIQGRSNYAEKFQG

SEQ ID NO: 286 - protein, 11 aa, synthetic construct
RTSENIYNNLA

SEQ ID NO: 287 - protein, 7 aa, synthetic construct
AATNLAD

SEQ ID NO: 288 - protein, 9 aa, synthetic construct
QHFWGTPLT

SEQ ID NO: 289 - protein, 7 aa, synthetic construct
AATNLAE

SEQ ID NO: 290 - protein, 10 aa, synthetic construct
QHFWGTPLTF

SEQ ID NO: 291 - protein, 11 aa, synthetic construct
RTSENIYSNLA

SEQ ID NO: 292 - protein, 7 aa, synthetic construct
AGTNLAD

SEQ ID NO: 293 - protein, 116 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 294 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 295 - protein, 116 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 296 - protein, 116 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 297 - protein, 116 aa, synthetic construct
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 298 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 299 - protein, 107 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 300 - protein, 107 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 301 - protein, 107 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

SEQ ID NO: 302 - protein, 107 aa, synthetic construct
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 303 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 304 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 305 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 306 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 307 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 308 - protein, 445 aa, synthetic construct
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 309 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 310 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 311 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 312 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 313 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 314 - protein, 445 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 315 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 316 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 317 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 318 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 319 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 320 - protein, 445 aa, synthetic construct
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 321 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 322 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCGVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 324 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 326 - protein, 445 aa, synthetic construct
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 327 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 328 - protein, 214 aa, synthetic construct
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 214 aa, synthetic construct
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 214 aa, synthetic construct
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 24 aa, synthetic construct
CGIFGEIEELIEEGLENLIDWGNA

SEQ ID NO: 332 - protein, 24 aa, synthetic construct
GLFEAIEGFIENGWEGMIDGWYGC

SEQ ID NO: 333 - protein, 27 aa, synthetic construct
GLFEAIEGFIENGWEGMIWDYGSGSCG

SEQ ID NO: 334 - protein, 23 aa, synthetic construct
  SITE 23: note = Glycine hexaethylene glycol amide
GLFEAIEGFIENGWEGMIDGWYG

SEQ ID NO: 335 - protein, 27 aa, synthetic construct
  CARBOHYD 27: note = Lysine N-linked N-acetylgalactosaminyl-(1->3)-N-acetylgalactosamine
GLFEAIEGFIENGWEGMIWDYGSGSCK

SEQ ID NO: 336 - protein, 27 aa, synthetic construct
CLIGAILKVLATGLPTLISWIKNKRKQ

SEQ ID NO: 337 - protein, 26 aa, synthetic construct
GIGAVLKVLTTGLPALISWIKRKRQQ

SEQ ID NO: 338 - protein, 13 aa, synthetic construct
  SITE 13: note = Phenylalanine amide
IFGAIAGLLKNIF

SEQ ID NO: 339 - protein, 18 aa, synthetic construct
FFGHLFKLATKIIPSLFQ

SEQ ID NO: 340 - protein, 21 aa, synthetic construct
KETWWETWWTEWSQPKKKRKV

SEQ ID NO: 341 - protein, 19 aa, synthetic construct
LLIILRRRRIRKQAHAHSK

SEQ ID NO: 342 - protein, 26 aa, synthetic construct
DPKGDPKGVTVTVTVTVTGKGDPKPD

SEQ ID NO: 343 - protein, 17 aa, synthetic construct
CSIPPEVKFNKPFVYLI

SEQ ID NO: 344 - protein, 27 aa, synthetic construct
GWTLNSAGYLLGKINLKALAALAKKIL

SEQ ID NO: 345 - protein, 21 aa, synthetic construct
AGYLLGKINLKALAALAKKIL

SEQ ID NO: 346 - protein, 17 aa, synthetic construct
GALFLGFLGAAGSTMGA

SEQ ID NO: 347 - protein, 20 aa, synthetic construct
HGLASTLTRWAHYNALIRAF

SEQ ID NO: 348 - protein, 20 aa, synthetic construct
GLWRALWRLLRSLWRLLWRA

SEQ ID NO: 349 - protein, 30 aa, synthetic construct
WEAALAEALAEALAEHLAEALAEALEALAA

SEQ ID NO: 350 - protein, 24 aa, synthetic construct
GLFEAIEGFIENGWEGMIDGWYGC

SEQ ID NO: 351 - protein, 23 aa, synthetic construct
GLFGAIAGFIENGWEGMIDGWYG

SEQ ID NO: 352 - protein, 36 aa, synthetic construct
  SITE 36: note = Lysine amide
GLFGAIAGFIENGWEGMIDGRQIKIWFQNRRMKWKK

SEQ ID NO: 353 - protein, 26 aa, synthetic construct
GLFGAIAGFIENGWEGMIDGSSKKKK

SEQ ID NO: 354 - protein, 24 aa, synthetic construct
GLFEAIAGFIENGWEGMIDGGGYC

SEQ ID NO: 355 - protein, 23 aa, synthetic construct
GLFHAIAHFIHGGWHGLIHGWYG

SEQ ID NO: 356 - protein, 30 aa, synthetic construct
  SITE 30: note = Alanine hexaethylene glycol amide
GLFEAIEGFIENGWEGLAEALAEALEALAA

SEQ ID NO: 357 - protein, 29 aa, synthetic construct
KWKLFKKIGAVLKVLTTGYGRKKRRQRRR

SEQ ID NO: 358 - protein, 16 aa, synthetic construct
RQIKIWFQNRRMKWKK

SEQ ID NO: 359 - protein, 13 aa, synthetic construct
GRKKRRQRRRPPQ

SEQ ID NO: 360 - protein, 18 aa, synthetic construct
LLIILRRRIRKQAHAHSK

SEQ ID NO: 361 - protein, 27 aa, synthetic construct
GWTLNSAGYLLGKINLKALAALAKKIL

SEQ ID NO: 362 - protein, 27 aa, synthetic construct
GALFLGFLGAAGSTMGAWSQPKKKRKV

SEQ ID NO: 363 - protein, 21 aa, synthetic construct
KETWWETWWTEWSQPKKKRKV

SEQ ID NO: 364 - protein, 12 aa, synthetic construct
  VARIANT 1..12: note = This sequence may encompass 6-12 residues
RRRRRRRRRRRR

SEQ ID NO: 365 - protein, 18 aa, synthetic construct
KLALKLALKALKAALKLA

SEQ ID NO: 366 - protein, 9 aa, synthetic construct
RRWWRRWRR

SEQ ID NO: 367 - protein, 13 aa, synthetic construct
CGYGPKKKRKVGG

SEQ ID NO: 368 - protein, 8 aa, synthetic construct
KKKKKKKK

SEQ ID NO: 369 - protein, 22 aa, synthetic construct
MVRRFLVTLRIRRACGPPRVRV

SEQ ID NO: 370 - protein, 28 aa, synthetic construct
LSTAADMQGVVTDGMASGLDKDYLKPDD

SEQ ID NO: 371 - DNA, 19 bp, synthetic construct
  source 1..19: mol_type = other DNA
ctgcctctccaccagccca

SEQ ID NO: 372 - DNA, 19 bp, synthetic construct
  source 1..19: mol_type = other DNA
tgggctggtggagaggcag

SEQ ID NO: 373 - DNA, 19 bp, synthetic construct
  source 1..19: mol_type = other DNA
gcagagatggagagaggaa

SEQ ID NO: 374 - DNA, 19 bp, synthetic construct
  source 1..19: mol_type = other DNA
ttcctctctccatctctgc

SEQ ID NO: 375 - DNA, 19 bp, synthetic construct
  source 1..19: mol_type = other DNA
gcggtttcctccgggacaa

SEQ ID NO: 376 - DNA, 19 bp, synthetic construct
  source 1..19: mol_type = other DNA
ttgtcccggaggaaaccgc

SEQ ID NO: 377 - DNA, 19 bp, synthetic construct
  source 1..19: mol_type = other DNA
ggacgacggaggcgtgatt

SEQ ID NO: 378 - DNA, 19 bp, synthetic construct
  source 1..19: mol_type = other DNA
aatcacgcctccgtcgtcc

SEQ ID NO: 379 - DNA, 25 bp, synthetic construct
  source 1..25: mol_type = other DNA
cgggcacccggaaacatgcagggaa

SEQ ID NO: 380 - DNA, 25 bp, synthetic construct
  source 1..25: mol_type = other DNA
ttccctgcatgtttccgggtgcccg

SEQ ID NO: 381 - DNA, 19 bp, synthetic construct
  source 1..19: mol_type = other DNA
ccggaaacatgcagggaag

SEQ ID NO: 382 - DNA, 19 bp, synthetic construct
  source 1..19: mol_type = other DNA
cttccctgcatgtttccgg

SEQ ID NO: 383 - DNA, 19 bp, synthetic construct
  source 1..19: mol_type = other DNA
gaaatgaacgagagccaca

SEQ ID NO: 384 - DNA, 19 bp, synthetic construct
  source 1..19: mol_type = other DNA
tgtggctctcgttcatttc

SEQ ID NO: 385 - DNA, 25 bp, synthetic construct
  source 1..25: mol_type = other DNA
tggcacactcaagactcccacggag

SEQ ID NO: 386 - DNA, 25 bp, synthetic construct
  source 1..25: mol_type = other DNA
ctccgtgggagtcttgagtgtgcca

SEQ ID NO: 387 - DNA, 19 bp, synthetic construct
  source 1..19: mol_type = other DNA
ccacggaggttcagttcca

SEQ ID NO: 388 - DNA, 19 bp, synthetic construct
  source 1..19: mol_type = other DNA
tggaactgaacctccgtgg

SEQ ID NO: 389 - DNA, 19 bp, synthetic construct
  source 1..19: mol_type = other DNA
accaccaccaccaccacca

SEQ ID NO: 390 - DNA, 19 bp, synthetic construct
  source 1..19: mol_type = other DNA
tggtggtggtggtggtggt

SEQ ID NO: 391 - DNA, 19 bp, synthetic construct
  source 1..19: mol_type = other DNA
cgccattcatgaaggggtg

SEQ ID NO: 392 - DNA, 19 bp, synthetic construct
  source 1..19: mol_type = other DNA
caccccttcatgaatggcg

SEQ ID NO: 393 - DNA, 19 bp, synthetic construct
  source 1..19: mol_type = other DNA
catgaaggggtggagcctg

SEQ ID NO: 394 - DNA, 19 bp, synthetic construct
  source 1..19: mol_type = other DNA
caggctccaccccttcatg

SEQ ID NO: 395 - DNA, 19 bp, synthetic construct
  source 1..19: mol_type = other DNA
gagcctgctttgagcggaa

SEQ ID NO: 396 - DNA, 19 bp, synthetic construct
  source 1..19: mol_type = other DNA
ttccgctcaaagcaggctc

SEQ ID NO: 397 - DNA, 25 bp, synthetic construct
  source 1..25: mol_type = other DNA
ccgagcctttgagaaggatcgcttt

SEQ ID NO: 398 - DNA, 25 bp, synthetic construct
  source 1..25: mol_type = other DNA
aaagcgatccttctcaaaggctcgg

SEQ ID NO: 399 - DNA, 19 bp, synthetic construct
  source 1..19: mol_type = other DNA
ggcagggcgcccgcgcagg

SEQ ID NO: 400 - DNA, 19 bp, synthetic construct
  source 1..19: mol_type = other DNA
cctgcgcgggcgccctgcc

SEQ ID NO: 401 - DNA, 19 bp, synthetic construct
  source 1..19: mol_type = other DNA
gatgattagttcagagata

SEQ ID NO: 402 - DNA, 19 bp, synthetic construct
  source 1..19: mol_type = other DNA
tatctctgaactaatcatc

SEQ ID NO: 403 - protein, 4 aa, synthetic construct
GGFG

SEQ ID NO: 404 - protein, 4 aa, synthetic construct
ALAL

SEQ ID NO: 405 - protein, 4 aa, synthetic construct
GFLG

SEQ ID NO: 406 - protein, 17 aa, synthetic construct
  VARIANT 6: note = N or Q
  VARIANT 13: note = Q or E
EINPIXGRSNYAXKFQG

SEQ ID NO: 407 - protein, 11 aa, synthetic construct
  VARIANT 8: note = N or S
RTSENIYXNLA

SEQ ID NO: 408 - protein, 7 aa, synthetic construct
  VARIANT 2: note = A or G
  VARIANT 7: note = D or E
AXTNLAX

SEQ ID NO: 409 - protein, 10 aa, synthetic construct
  VARIANT 10: note = F or absent
QHFWGTPLTX

SEQ ID NO: 410 - protein, 7 aa, synthetic construct
  VARIANT 7: note = D or E
AATNLAX

SEQ ID NO: 411 - skipped

SEQ ID NO: 412 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tacgagtctccgtcgccgttt

SEQ ID NO: 413 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylcytidine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tccaaacgagtctccgtcgtt

SEQ ID NO: 414 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluoroadenosine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
taagcgatccttctcaaagtt

SEQ ID NO: 415 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tagcgagctcccttgcacgtt

SEQ ID NO: 416 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methylcytidine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tcctagacagcgtcggaagtt

SEQ ID NO: 417 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorouridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methyladenosine phosphorothioate
  modified_base 20: mod_base = OTHER; note = 2'-O-methyluridine phosphorothioate
  modified_base 21: mod_base = OTHER; note = 2'-O-methyluridine
ttgcctagacagcgtcggatt

SEQ ID NO: 418 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorouridine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methyluridine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tttgcctagacagcgtcggtt

SEQ ID NO: 419 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluoroguanosine phosphorothioate
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methyluridine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tggtttgcctagacagcgttt

SEQ ID NO: 420 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorouridine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methylcytidine
  modified_base 8: mod_base = OTHER; note = 2'-O-methyluridine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroadenosine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
ttaactctaatccaggttttt

SEQ ID NO: 421-429 - skipped (9 entries)

SEQ ID NO: 430 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluoroadenosine phosphorothioate
  modified_base 3: mod_base = OTHER; note = 2'-O-methylcytidine
  modified_base 4: mod_base = OTHER; note = 2'-O-methylguanosine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyluridine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tacgagtctccgtcgccgttt

SEQ ID NO: 431 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methylcytidine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylcytidine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tccaaacgagtctccgtcgtt

SEQ ID NO: 432 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluoroadenosine phosphorothioate
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methylcytidine
  modified_base 11: mod_base = OTHER; note = 2'-O-methyluridine
  modified_base 12: mod_base = OTHER; note = 2'-O-methyluridine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
taagcgatccttctcaaagtt

SEQ ID NO: 433 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluoroadenosine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylguanosine
  modified_base 6: mod_base = OTHER; note = 2'-fluoroadenosine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methyluridine
  modified_base 10: mod_base = OTHER; note = 2'-O-methylcytidine
  modified_base 11: mod_base = OTHER; note = 2'-O-methylcytidine
  modified_base 12: mod_base = OTHER; note = 2'-O-methylcytidine
  modified_base 13: mod_base = OTHER; note = 2'-O-methyluridine
  modified_base 14: mod_base = OTHER; note = 2'-fluorouridine
  modified_base 15: mod_base = OTHER; note = 2'-O-methylguanosine
  modified_base 16: mod_base = OTHER; note = 2'-fluorocytidine
  modified_base 17: mod_base = OTHER; note = 2'-O-methyladenosine
  modified_base 18: mod_base = OTHER; note = 2'-O-methylcytidine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tagcgagctcccttgcacgtt

SEQ ID NO: 434 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluorocytidine phosphorothioate
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-fluoroguanosine
  modified_base 7: mod_base = OTHER; note = 2'-O-methyladenosine
  modified_base 8: mod_base = OTHER; note = 2'-O-methylcytidine
  modified_base 9: mod_base = OTHER; note = 2'-O-methyladenosine
  modified_base 10: mod_base = OTHER; note = 2'-O-methylguanosine
  modified_base 11: mod_base = OTHER; note = 2'-O-methylcytidine
  modified_base 12: mod_base = OTHER; note = 2'-O-methylguanosine
  modified_base 13: mod_base = OTHER; note = 2'-O-methyluridine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tcctagacagcgtcggaagtt

SEQ ID NO: 435 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluorouridine phosphorothioate
  modified_base 3: mod_base = OTHER; note = 2'-O-methylguanosine
  modified_base 4: mod_base = OTHER; note = 2'-O-methylcytidine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methylguanosine
  modified_base 9: mod_base = OTHER; note = 2'-O-methyladenosine
  modified_base 10: mod_base = OTHER; note = 2'-O-methylcytidine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methylcytidine
  modified_base 14: mod_base = OTHER; note = 2'-fluoroguanosine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methylguanosine
  modified_base 18: mod_base = OTHER; note = 2'-O-methylguanosine
  modified_base 19: mod_base = OTHER; note = 2'-O-methyladenosine phosphorothioate
  modified_base 20: mod_base = OTHER; note = 2'-O-methyluridine phosphorothioate
  modified_base 21: mod_base = OTHER; note = 2'-O-methyluridine
ttgcctagacagcgtcggatt

SEQ ID NO: 436 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluorouridine phosphorothioate
  modified_base 3: mod_base = OTHER; note = 2'-O-methyluridine
  modified_base 4: mod_base = OTHER; note = 2'-O-methylguanosine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methyluridine
  modified_base 8: mod_base = OTHER; note = 2'-O-methyladenosine
  modified_base 9: mod_base = OTHER; note = 2'-O-methylguanosine
  modified_base 10: mod_base = OTHER; note = 2'-O-methyladenosine
  modified_base 11: mod_base = OTHER; note = 2'-O-methylcytidine
  modified_base 12: mod_base = OTHER; note = 2'-O-methyladenosine
  modified_base 13: mod_base = OTHER; note = 2'-O-methylguanosine
  modified_base 14: mod_base = OTHER; note = 2'-fluorocytidine
  modified_base 15: mod_base = OTHER; note = 2'-O-methylguanosine
  modified_base 16: mod_base = OTHER; note = 2'-fluorouridine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methylguanosine phosphorothioate
  modified_base 20: mod_base = OTHER; note = 2'-O-methyluridine phosphorothioate
  modified_base 21: mod_base = OTHER; note = 2'-O-methyluridine
tttgcctagacagcgtcggtt

SEQ ID NO: 437 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluoroguanosine phosphorothioate
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methyluridine
  modified_base 6: mod_base = OTHER; note = 2'-fluorouridine
  modified_base 7: mod_base = OTHER; note = 2'-O-methylguanosine
  modified_base 8: mod_base = OTHER; note = 2'-O-methylcytidine
  modified_base 9: mod_base = OTHER; note = 2'-O-methylcytidine
  modified_base 10: mod_base = OTHER; note = 2'-O-methyluridine
  modified_base 11: mod_base = OTHER; note = 2'-O-methyladenosine
  modified_base 12: mod_base = OTHER; note = 2'-O-methylguanosine
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methylcytidine
  modified_base 18: mod_base = OTHER; note = 2'-O-methylguanosine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
tggtttgcctagacagcgttt

SEQ ID NO: 438 - RNA, 21 nt, synthetic construct
  source 1..21: mol_type = other RNA
  modified_base 1: mod_base = OTHER; note = vinyl phosphonate vpUq uracil phosphorothioate
  modified_base 2: mod_base = OTHER; note = 2'-fluorouridine phosphorothioate
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methylcytidine
  modified_base 8: mod_base = OTHER; note = 2'-O-methyluridine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyluridine
  modified_base 12: mod_base = OTHER; note = 2'-O-methylcytidine
  modified_base 13: mod_base = OTHER; note = 2'-O-methylcytidine
  modified_base 14: mod_base = OTHER; note = 2'-fluoroadenosine
  modified_base 15: mod_base = OTHER; note = 2'-O-methylguanosine
  modified_base 16: mod_base = OTHER; note = 2'-fluoroguanosine
  modified_base 17: mod_base = OTHER; note = 2'-O-methyluridine
  modified_base 18: mod_base = OTHER; note = 2'-O-methyluridine
  modified_base 19: mod_base = OTHER; note = 2'-O-methyluridine phosphorothioate
  modified_base 20: mod_base = OTHER; note = 2'-O-methyluridine phosphorothioate
  modified_base 21: mod_base = OTHER; note = 2'-O-methyluridine
ttaactctaatccaggttttt